(19) United States
(12) Patent Application Publication
(10) Pub. No.: US 2005/0009903 A1
(43) Pub. Date: Jan. 13, 2005

(54) CB2-selective cannabinoid analogues

(76) Inventors: Billy R. Martin, Richmond, VA (US); John W. Huffman, Clemson, NC (US)

Correspondence Address: WHITHAM, CURTIS & CHRISTOFFERSON, P.C., 11491 SUNSET HILLS ROAD, SUITE 340, RESTON, VA, 20190, US

(21) Appl. No.: 10/864,819

(22) Filed: Jun. 10, 2004

Related U.S. Application Data
(60) Provisional application No. 60/555,355, filed on Mar. 23, 2004.
(60) Provisional application No. 60/477,044, filed on Jun. 10, 2003.

(51) Int. Cl. C07D 311/92; A61K 31/353

(52) U.S. Cl. 514/454; 549/389

(57) ABSTRACT

Cannabinoid analogues that exhibit specificity for the CB₂ cannabinoid receptor are provided. The analogues are 1-methoxy-, 1-deoxy-11-hydroxy- and 11-hydroxy-1-methoxy-Δ⁸-tetrahydrocannabinols and 1-alkyl-3(1-naphthoyl)indoles. The compounds are useful for the treatment of pain (especially pain resulting from inflammation) and cancer (especially glioma tumors).

This invention was made using funds from grants from the National Institutes of Health having grant numbers DA 03672 and DA 03590. The United States government may have certain rights in this invention.

### BACKGROUND OF THE INVENTION

1. Field of the Invention

The invention generally relates to cannabinoid analogues. In particular, the invention provides cannabinoid analogues that exhibit specificity for the CB₂ cannabinoid receptor.

2. Background of the Invention

The complex effects of cannabinoids are considered to be mediated through at least two G-protein coupled, transmembrane receptors. One of these, designated as CB₁, is found predominantly in the central nervous system and is responsible for most of the overt pharmacological effects of cannabinoids. A second receptor, designated CB₂ was originally identified from macrophages present in the spleen, and is expressed primarily in the periphery. Although it has been known for some time that cannabinoids are involved in immunomodulation, the discovery that CB₂ receptors are expressed primarily in the immune system led to the suggestion that the CB₂ receptor was responsible for the immunomodulatory effects of cannabinoids. This suggestion has recently been confirmed by the observation that such effects are absent in CB₂ receptor knockout mice. Furthermore, recent studies have now highlighted other beneficial effects specific to the CB₂ receptor. These effects include the discovery that the CB₂ selective ligand, JWH-133, is effective in reducing spasticity in the mouse model of multiple sclerosis. More pertinently, the same CB₂ selective ligand inhibits the in vivo growth of glioma tumors. Other effects modulated by the CB₂ receptor include peripheral antinociception and, at least in part, the antitumor properties of ajulemic acid.

There is an ongoing need for the development of ligands which are specific for the CB₂ receptor.

### SUMMARY OF THE INVENTION

It is an object of this invention to provide cannabinoid analogues which bind preferentially to the CB₂ cannabinoid receptor, and which exhibit little or no binding affinity for the CB₁ receptor. The analogues are 1-methoxy-, 1-deoxy-11-hydroxy- and 11-hydroxy-1-methoxy-Δ⁸-tetrahydrocannabinols and 1-alkyl-3(1-naphthoyl)indoles.

In one embodiment, the invention provides a compound of general formula 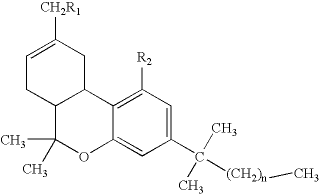
wherein R₁ and R₂ are H, OH or OCH₃; n ranges from 0 to 4; and R₁ and R₂ are not both H. A steric representation of this general formula is 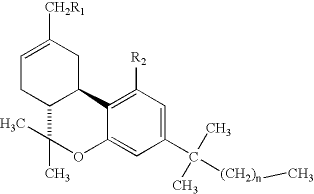
where R₁ is H or OH; R₂ is H or OCH₃; n ranges from 0 to 4; and R₁ and R₂ are not both H. In one embodiment of the compound, R₁ is H and R₂ is OCH₃. In another embodiment, R₁ is OH and R₂ is H. In yet another embodiment, R₁ is OH and R₂ is OCH₃.

The invention further provides a method for selectively binding to CB₂ receptors and not to CB₁ receptors. The method comprises the step of exposing the CB₂ receptors and the CB₁ receptors to a compound of general formula 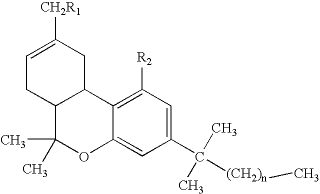
wherein R₁ and R₂ are H, OH, or OCH₃; n ranges from 0 to 4; and R₁ and R₂ are not both H. One possible steric representation of the general formula is 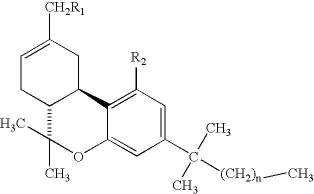
where R₁ is H or OH; R₂ is H or OCH₃; n ranges from 0 to 4; and R₁ and R₂ are not both H. In preferred embodiments: R₁ is H and R₂ is OCH₃; or R₁ is OH and R₂ is H; or R₁ is OH and R₂ is OCH₃.

In one embodiment of the invention, the compound binds to the CB₂ receptors in an amount sufficient to treat cancer, for example, a glioma tumor. In another embodiment, the compound binds to the CB₂ receptors in an amount sufficient to treat pain, e.g. inflammatory pain.

The present invention provides a method for killing tumor cells. The method includes the step of exposing the tumor cells to a compound of general formula 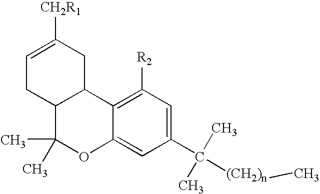
wherein R₁ and R₂ are H, OH, or OCH₃; n ranges from 0 to 4; and R₁ and R₂ are not both H. One possible steric representation of the general formula is 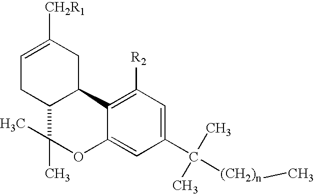
where R₁ is H or OH; R₂ is H or OCH₃; n ranges from 0 to 4; and R₁ and R₂ are not both H. In preferred embodiments: R₁ is H and R₂ is OCH₃; or R₁ is OH and R₂ is H; or R₁ is OH and R₂ is OCH₃. The tumor cells may be glioma tumor cells.

The invention further provides a compound of general formula 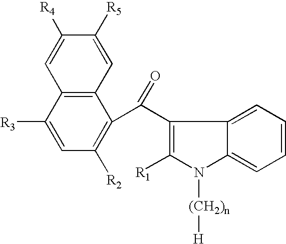
wherein R₁, R₂, R₃, R₄ and R₅ are H, CH₃, or OCH₃; and n ranges from 1 to 5. In some embodiments of the invention: R₁ is H or CH₃; R₂ is H or OCH₃; R₃ is H, CH₃, or OCH₃; R4 is H or OCH₃; and R₅ is H or CH₃. Examples of such compounds include those where: 1) n is 3; R₁, R₃, R₄ and R₅ are H; and R₂ is OCH₃; 2) n is 3; R₁ is CH₃; R₂ is OCH₃; and R₃, R₄ and R₅ are H; 3) n is 5; R₁, R₃, R₄ and R₅ are H; and R₂ is OCH₃; 4) n is 5; R₁ is CH₃, R₂ is OCH₃ and R₃, R₄ and R₅ are H; 5) n is 3 or 5; R₁, R₂, R₃ and R₅ are H; and R₄ is OCH₃; 6) n is 3 or 5; R₁ is CH₃; R₂, R₃ and R₅ are H; and R₄ is OCH₃; 7) n is 3; R₁, R₂, R₄ and R₅ are H; and R₃ is CH₃; and 8) n is 3 or 5; R₁ is CH₃;R₂, R₃ and R₄ are H; and R₅ is CH₃.

The invention also provides a method for selectively binding toCB₂ receptors and not to CB₁ receptors. The method includes the step of exposing the CB₂ receptors and the CB₁ receptors to a compound of general formula 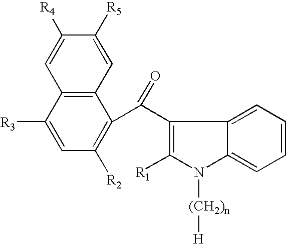
where R₁, R₂, R₃, R₄ and R₅ are H, CH₃, or OCH₃; and n ranges from 1 to 5. In some embodiments: R₁ is H or CH₃; R₂ is H or OCH₃;R₃ is H, CH₃, or OCH₃; R4 is H or OCH₃; and R₅ is H or CH₃. In preferred embodiments: 1) n is 3; R₁, R₃,R₄ and R₅ are H; and R₂ is OCH₃; 2) n is 3; R₁ is CH₃;R₂ is OCH₃; and R₃, R₄ and R₅ are H; 3) n is 5; R₁, R₃, R₄ and R₅ are H; and R₂ is OCH₃; 4) n is 5; R₁ is CH₃, R₂ is OCH₃ and R₃, R₄ and R₅ are H; 5) n is 3 or 5; R₁, R₂, R₃ and R₅ are H; and R₄ is OCH₃; 6) n is 3 or 5; R₁ is CH₃; R₂, R₃ and R₅ are H; and R₄ is OCH₃; 7) n is 3; R₁, R₂, R₄ and R₅ are H; and R₃ is CH₃; and 8) n is 3 or 5; R₁ is CH₃; R₂, R₃ and R₄ are H; and R₅ is CH₃.

The compound binds to the CB₂ receptors in an amount sufficient to treat cancer, e.g. a glioma tumor. Alternatively, the compound binds to the CB₂ receptors in an amount sufficient to treat pain, e.g. inflammatory pain.

The present invention also provides a method for killing tumor cells. The method includes the step of exposing the tumor cells to a compound of general formula 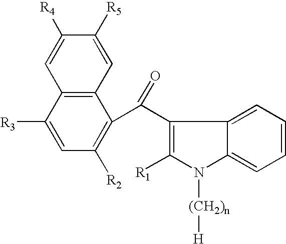
where R₁, R₂, R₃, R₄ and R₅ are H, CH₃, or OCH₃; and n ranges from 1 to 5.

In some embodiments: R₁ is H or CH₃; R₂ is H or OCH₃;R₃ is H, CH₃, or OCH₃; R4 is H or OCH₃; and R₅ is H or CH₃. In preferred embodiments: 1) n is 3; R₁, R₃, R₄ and R₅ are H; and R₂ is OCH₃; 2) n is 3; R₁ is CH₃; R₂ is OCH₃; and R₃, R₄ and R₅ are H; 3) n is 5; R₁, R₃, R₄ and R₅ are H; and R₂ is OCH₃; 4) n is 5; R₁ is CH₃, R₂ is OCH₃ and R₃, R₄ and R₅ are H; 5) n is 3 or 5; R₁, R₂, R₃ and R₅ are H; and R₄ is OCH₃; 6) n is 3 or 5; R₁ is CH₃; R₂, R₃ and R₅ are H; and R₄ is OCH₃; 7) n is 3; R₁, R₂, R₄ and R₅ are H; and R₃ is CH₃; and 8) n is 3 or 5; R₁ is CH₃; R₂, R₃ and R₄ are H; and R₅ is CH₃.

In a preferred embodiment, the tumor cells are glioma tumor cells.

### BRIEF DESCRIPTION OF THE DRAWINGS

FIG. 1. Depictions of (1) 1-deoxy-11-hydroxy-Δ⁸-THC-DMH,deoxy-HU-210, JWH-051; (2) 1-deoxy-Δ⁸-THC-DMH, JWH-057; (3) 3-(1′,1′-dimethylbutyl)-1-deoxy-Δ⁸-THC, JWH-133; (4) 1-methoxy-Δ⁸-THC-DMH; and 1-methoxy-66 ⁹⁽¹¹⁾-THC-DMH (5).

FIG. 2. Synthesis scheme. (a) HOTs/C₆H₆, 80° C.; (b) CH₃ I/KOH/DMF, 25° C.; c) NaH/THF, 0° C. then (C₂H₅O)₂P(O)Cl; (d) Li/NH₃, THF, −78° C.

FIG. 3. Synthesis scheme. a) SeO₂/EtOH, 80° C.; b) LiAlH₄/THF, 25° C. or NaBH₄/CeCl₃. 7H₂O/MeOH.

FIG. 4. Synthesis scheme. a) BF₃.Et₂O/CH₂Cl₂, −20° C.; b) CH₃I/KOH/DMF, 25° C.; c) LiAlH₄/THF, 25° C. or NaBH₄/CeCl₃.7H₂O/MeOH.

### DETAILED DESCRIPTION OF THE PREFERRED EMBODIMENTS OF THE INVENTION

The present invention provides compounds that bind with high affinity to the CB₂ cannabinoid receptor and methods for their use. By binding with “high affinity” we mean that the Kᵢ values of the compounds for the CB₂ receptor are in the range of about 0.03 nM to about 40 nM. In a preferred embodiment, the compounds display both high binding affinity for the CB₂ receptor and high binding selectivity for the CB₂ receptor. By “binding selectivity” we mean that the compounds exhibit an affinity for the CB₂ cannabinoid receptor that is in the range of about 10 to about 200 fold or more higher than their affinity for the CB₁ cannabinoid receptors. In other words, the Kᵢ values of the compounds for CB₂ receptors is in the range of about 10 to about 200 times or more lower than the Kᵢ values of the compounds for the CB₁ receptor. Measurement of the Kᵢ values may be carried out in any of several known assay systems, including but not limited to biological tissue such as brain and spleen, or in cells that are genetically modified to express CB₁ or CB₂ receptors. In a preferred embodiment, the assay system is brain tissue that contains CB₁ receptors and cells genetically modified to express CB₂ receptors.

In one embodiment of the invention, the compounds are of the general formula represented in Formula 1 below. 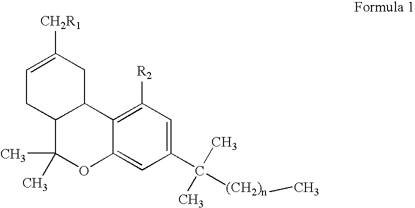
In Formula 1, R₁ and R₂ may be H, OH or OCH₃, but R₁ and R₂ are not both H; and n may range from 0 to 4. In some embodiments of the invention, R₁ is H or OH and R₂ is H or OCH₃, In preferred embodiments of the invention:
a) R₁ is H, R₂ is OCH₃ and n is 3 or 4;
b) R₁ is OH, R₂ is H and n is 0 or 1; and
c) R₁ is OH, R₂ is OCH₃ and n is 2.
   Compounds of type a-c, exhibit both high affinity and high selectivity for the CB₂ cannabinoid receptor.

In another embodiment of the invention, the compounds of the invention are of the general formula represented in Formula 2 below. 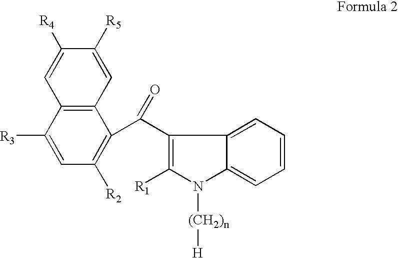
In Formula 2, R₁, R₂, R₃, R₄ and R₅ are H, CH₃, or OCH₃; and n ranges from 1 to about 5. In some embodiments, R₁ is H or CH₃; R₂ is H or OCH₃; R₃ is H, CH₃, or OCH₃; R₄ is H or OCH₃; and R₅ is H or CH₃. In preferred embodiments of the invention: n is 3 or 5, R₁ is H or CH₃, R₂ is OCH₃ and R₄ and R₅ are H (e.g. compounds 30-33 of Example 2, listed in Table 3, where:
i. for compound 30, n is 3; R₁ and R₃ are H; and R₂ is OCH₃;
ii. for compound 31, n is 3; R₁ is CH₃; R₂ is OCH₃; and R₃ is H;
iii. for compound 32, n is 5; R₁ and R₂ are H; and R₂ is OCH₃; and
iv. for compound 33, n is 5; R₁ is CH₃; R₂ is OCH₃; and R₃ is H.
   In other preferred embodiments, n is 3 or 5; R₁ is H or CH₃; R₂, R₃ and R₅ are H; and R₄ is OCH₃(see Table 3, compounds JWH-163, JWH-151 (42), JWH-166, and JWH-153, where:
i. for JWH-163, n=3; R₁, R₂, R₃ and R₅ are H; and R₄ is OCH₃;
ii. for JWH-151 (42), n=3; R₁ is CH₃; R₂, R₃ and R₅ are H; and R₄ is OCH₃;
iii. for JHW-166, n=5; R₁, R₂, R₃ and R₅ are H; and R₄ is OCH₃; and
iv. For JHW-153, n=5; R₁ is CH₃; R₂, R₃ and R₅ are H; and R₄ is OCH₃.
   In yet other preferred embodiments:
n=3; R₁, R₂, R₄ and R₅ are H; and R₃ is CH₃(compound JWH-120, 41, as depicted in Table 2 of Example 2); and
n=3 or 5; R₁ is CH₃; R₂, R₃ and R₄ are H; and R₅ is CH₃(compounds JWH-046, 8, and JWH-048, 9, as depicted in Table 2 of Example 2).

The compounds of the present invention may be useful as analgesics in a variety of applications including but not limited to pain management. Pain of any type may be ameliorated by the administration of the compounds of the present invention. In preferred embodiments, the compounds of the present invention are administered to treat acute and chronic inflammatory pain (e.g., inflammation from autoimmune disorders such as arthritis, inflammation from injuries or from surgery, etc.).

In addition, the compounds of the present invention may be used for the treatment of cancer. Examples of types of cancer that may be treated by the administration of the present compounds include but are not limited to glioma tumors and non-melanoma skin cancers. In a preferred embodiment, the compounds are used to treat glioma tumors.

The invention thus provides a method for treating cancer in a patient in need thereof. By “treating” cancer we mean that the compound is administered in order to alleviate symptoms of the disease (e.g. to decrease tumor size, halt metastasis, etc.). Those of skill in the art will recognize that, in the treatment of cancer, administration of the compounds of the present invention may completely eradicate symptoms of the disease, or, alternatively, may attenuate or slow the progression of disease, which is also beneficial to the patient.

While in preferred embodiments, the compounds of the present invention may be used to treat inflammatory pain and cancer, in a more general sense, the invention further provides methods of treating conditions or disorders related to CB₂ receptor-regulated systems. By “CB₂receptor-regulated systems” we mean biochemical pathways which include the binding of a cannabinoid to the CB₂ receptor molecule within the pathway. Examples of such biochemical pathways include but are not limited to activation of G-proteins and subsequent inhibition of adenylyl cyclase. The compounds of the present invention may be used to treat aberrations in CB₂ receptor-regulated systems, or to alter CB₂ receptor-regulated systems when it is desirable to do so in order to ease untoward symptoms in a patient. For example, patients with pain or spasticity arising from inflammation or neuropathological damage will benefit from activation of the CB₂ receptor with a selective agonist. In addition, the compounds may also be used for research purposes.

Implementation of the claimed invention will generally involve identifying patients suffering from the indicated disorders and administering the compounds of the present invention in an acceptable form by an appropriate route. The exact dosage to be administered may vary depending on the age, gender, weight and overall health status of the individual patient, as well as the precise etiology of the disease. However, in general for administration in mammals (e.g. humans), dosages in the range of from about 0.1 to about 30 mg of compound per kg of body weight per 24 hr., and more preferably about 0.1 to about 10 mg of compound per kg of body weight per 24 hr., are effective.

Administration may be oral or parenteral, including intravenously, intramuscularly, subcutaneously, intradermal injection, intraperitoneal injection, etc., or by other routes (e.g. transdermal, sublingual, oral, rectal and buccal delivery, inhalation of an aerosol, etc.).

The compounds may be administered in the pure form or in a pharmaceutically acceptable formulation including suitable elixirs, binders, and the like (generally referred to a “carriers”) or as pharmaceutically acceptable salts (e.g. alkali metal salts such as sodium, potassium, calcium or lithium salts, ammonium, etc.) or other complexes. It should be understood that the pharmaceutically acceptable formulations include liquid and solid materials conventionally utilized to prepare both injectable dosage forms and solid dosage forms such as tablets and capsules and aerosolized dosage forms. In addition, the compounds may be formulated with aqueous or oil based vehicles. Water may be used as the carrier for the preparation of compositions (e.g. injectable compositions) which may also include conventional buffers and agents to render the composition isotonic. Other potential additives and other materials (preferably those which are generally regarded as safe [GRAS]) include: colorants; flavorings; surfactants (TWEEN, oleic acid, etc.); solvents, elixirs, and binders or encapsulants (lactose, liposomes, etc). Solid diluents and excipients include lactose, starch, conventional disintergrating agents, coatings and the like. Preservatives such as methyl paraben or benzalkium chloride may also be used. Depending on the formulation, it is expected that the active composition will consist of about 1% to about 99% of the composition and the vehicular “carrier” will constitute about 1% to about 99% of the composition. The pharmaceutical compositions of the present invention may include any suitable pharmaceutically acceptable additives or adjuncts to the extent that they do not hinder or interfere with the therapeutic effect of the active compound.

The administration of the compounds of the present invention may be intermittent, or at a gradual or continuous, constant or controlled rate to a patient. In addition, the time of day and the number of times per day that the pharmaceutical formulation is administered may vary are and best determined by a skilled practitioner such as a physician. Further, the effective dose can vary depending upon factors such as the mode of delivery, gender, age, and other conditions of the patient, as well as the extent or progression of the disease. The compounds may be provided alone, in a mixture containing two or more of the compounds, or in combination with other medications or treatment modalities. The compounds may also be added to blood ex vivo and then be provided to the patient.

### EXAMPLES

### Example 1

### 1-Methoxy-, 1-Deoxy-11-hydroxy- and 11-Hydroxy-1-methoxy-Δ⁸-tetrahydrocannabinols: New Selective Ligands for the CB₂ Receptor Introduction

The complex pharmacological effects of cannabinoids are considered to be mediated through at least two G-protein-coupled, transmembrane receptors. One of these, designated as CB₁, is found predominantly in the central nervous system and is responsible for most of the overt pharmacological effects of cannabinoids.¹⁻⁴ A second receptor, designated CB₂, was originally identified from macrophages present in the spleen, and is expressed primarily in the periphery.⁵ Very recently evidence has been presented for the existence of a third cannabinoid receptor, which has been detected in mouse brain.⁶ It is generally accepted that the CB₁ receptor is implicated in eliciting the in vivo effects of cannabinoids; a good correlation has been found between the CB₁ receptor affinities of a series of cannabinoids and their in vivo effects.^{7,8} These in vivo effects are blocked by SR141716A, an inverse agonist for the CB₁ receptor, and are absent in CB₁ receptor knockout mice.^{9,10}

Although it has been known for some time that cannabinoids are involved in immunomodulation,¹¹ the discovery that the CB₂ receptor is expressed primarily in cells of the immune system led to the suggestion that the CB₂ receptor was responsible for the immunomodulatroy effects of cannabinoids.⁵ This suggestion has been confirmed recently by the observation that these immunomodulatory effects are absent in CB₂ receptor knockout mice.¹² Although there is evidence that the CB₂ receptor is not expressed in the central nervous system,¹³ it has recently been found that this receptor is expressed in adult rat retina.¹⁴

Although it has been known for several years that the CB₂ receptor is expressed in cells in the immune system, it has only been within the past few years that specific effects mediated by this receptor have been recognized. These effects included the discovery that a CB₂ selective receptor ligand, JWH-133, is effective in reducing spasticity in the mouse model of multiple sclerosis,¹⁵ and the same CB₂ selective ligand also inhibits the in vivo growth of glioma tumors.¹⁶ Other effects modulated by the CB₂ receptor include peripheral antinociception,¹⁷ and at least in part, the antitumor properties of ajulemic acid.¹⁸

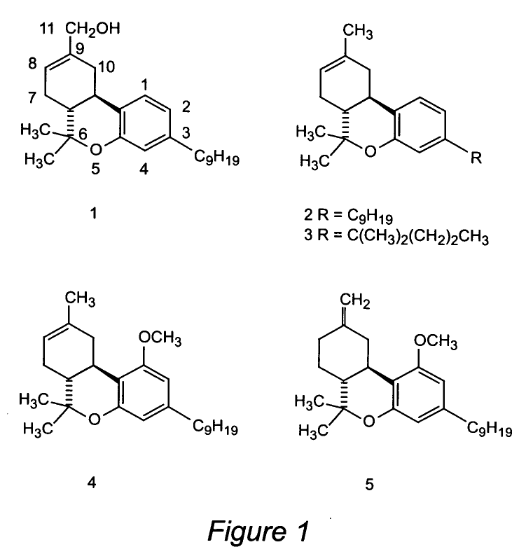

Several years ago we reported that 3-(1′,1′-dimethyl-heptyl)-1-deoxy-11-hydroxy-66 ⁸-tetrahydrocannabinol (1-deoxy-11-hydroxy-Δ⁸-THC-DMH,deoxy-HU-210,JWH-051 (1 in FIG. 1), a traditional cannabinoid lacking a 1-hydroxyl group, has very high affinity for the CB₁ receptor (Kᵢ=1.2 0.1 nM), and exhibits characteristic cannabinoid in vivo pharmacology. Cannabinoid 1 also has exceptionally high affinity for the CB₂ receptor (Kᵢ=0.032 0.019 nM).¹⁹ A second 1-deoxycannabinoid, 3-(1′,1′-dimethylheptyl)-1-deoxy-Δ⁸-THC (1-deoxy-Δ⁸-THC-DMH,JWH-057, 2 in FIG. 1), is also potent in vivo, has significant affinity for the CB₁ receptor (Kᵢ=23 7 nM), and nearly ten times greater affinity for the CB₂ receptor (Kᵢ=2.9 1.6 nM).¹⁹ Based on these observations, we synthesized a number of 1-deoxy-Δ⁸-THC analogues.²⁰ Several of these compounds have high affinity for the CB₂ receptor, with low affinity for the CB₁ receptor, and one of them, 3-(1′,1′-dimethylbutyl)-1-deoxy-Δ⁸-THC (JWH-133, 3 in FIG. 1) with Kᵢ=3.430 1.0 nM at CB₂ and 677±132 nM at CB₁ is highly selective. In the same publication, we developed some preliminary structure-activity relationships (SAR) for the CB₂ receptor. These preliminary SAR demonstrated that a 1′,1′-dimethyl group leads to enhanced affinity for the CB₂ receptor, and that in the 10, 10-dimethyl-1-deoxy-Δ⁸-THC series, compounds with a three to seven carbon side chain all have high affinity for the CB₂ receptor (K, <20 nM). Also, affinity for both receptors is enhanced by the presence of an 11-hydroxyl group.

Others have described two 1-methoxy cannabinoids,1-methoxy-Δ⁸-THC-DMH (4 in FIG. 1), and 1-methoxy-66 ⁹⁽¹¹⁾-THC-DMH (5 in FIG. 1) which were reported to have affinities for the CB₂ receptor in the 20 nM range, and virtually no affinity for the CB₁ receptor.²¹ An additional CB₂ selective agonist, HU-308 was reported by Hanus et al.; this compound has no affinity for the CB₁ receptor (Kᵢ>10,000 nM), and good affinity for the CB₂ receptor (Kᵢ=22.7±3.9 nM).²² HU-308 is inactive in the mouse behavioral tetrad, reduces blood pressure and shows peripheral analgesic activity. These hypotensive and analgesic effects are blocked by SR-1 44528, a CB₂ antagonist.

In view of the continuing recognition of the importance of the CB₂ receptor, we have taken advantage of the currently available knowledge of the SAR of 1-methoxy-and 1-deoxy-66 ⁸-THC analogues to design three series of CB₂ selective cannabinoid receptor ligands. Based on the knowledge that the side chain in the 1-deoxy-Δ⁸-THC series can be shortened significantly without seriously attenuating CB₂ receptor affinity, and that an 11-hydroxyl enhances both CB₁ and CB₂ receptor affinities,²⁰ the initial synthetic targets included a series of 1-deoxy-3-(10,10-dimethylalkyl)-11-hydroxy-Δ⁸-THC analogues. Since 1-methoxy-Δ⁸-THC-DMH (4) is also a CB₂ selective cannabinoid ligand,^{20,21,23} a series of 3-(1′,1′-dimethylalkyl)-1-methoxy-Δ⁸-THCs was also prepared. The third series of CB₂ selective compounds consisted of 11-hydroxy-3-(1′,1′-dimethylalkyl)-1-meth-oxy-Δ⁸-THC analogues which combined the structural features of the other two series.

Results

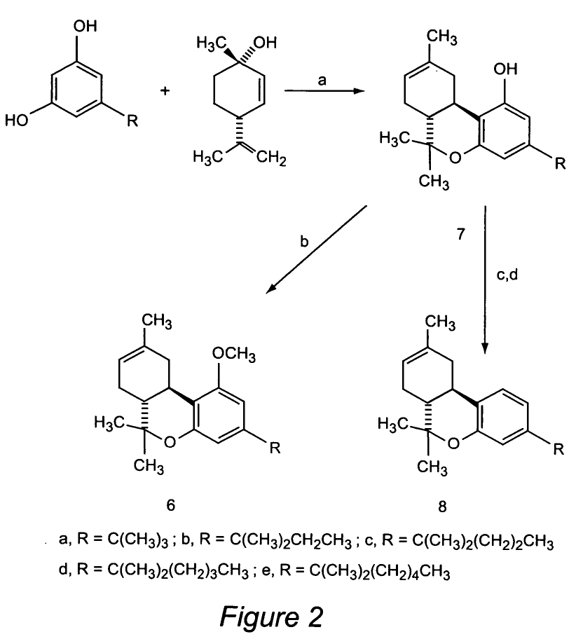

The 1-methoxy-Δ⁸-THC analogues (6a-6e, FIG. 2) were prepared by direct methylation of the corresponding Δ⁸-THC (7a-7e, FIG. 2) using methyl iodide/KOH in DMF in unoptimized yields of 43-97%. The cannabinoid substrates are all known compounds which were prepared by the acid catalyzed condensation of the corresponding alkyl resorcinol with trans-p-menthadienol.^{20,24,25}

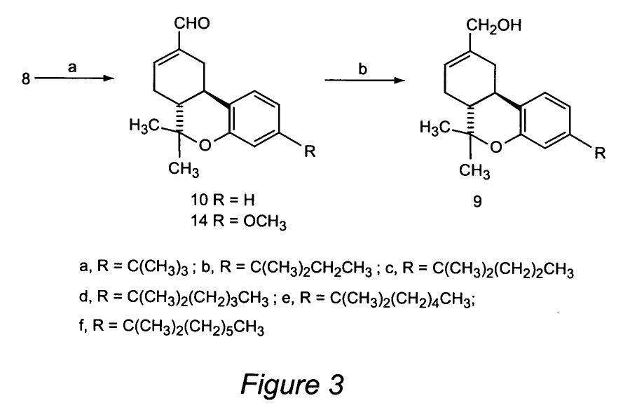

The 1-deoxy-11-hydroxy-Δ⁸-THCs (9a-9e, FIG. 3) were prepared from the corresponding 1-deoxy-Δ⁸-THC (8a-8e, FIG. 2) by initial selenium dioxide oxidation to the 11-oxo compounds (10a-10e, FIG. 3), followed by reduction of the aldehyde. The requisite 1-deoxy-Δ⁸-THCs (8a-8e, FIG. 2) were prepared from the corresponding Δ⁸-THC by conversion to the phosphate ester, followed by dissolving metal reduction using procedures we have described previously.²⁰Selenium dioxide oxidation of cannabinoids 8a-8e (FIG. 2) using a previously developed procedure provided aldehydes 10a-10e (FIG. 3),²⁶ which were reduced to the 11-hydroxy analogues without extensive purification. Lithium aluminum hydride reduction of aldehydes 10a-10e (FIG. 3) provided alcohols 9a-9e (FIG. 3) in modest yields for the two steps. Improved yields of alcohols 9 were obtained by the method of Luche (sodium borohydride-cerium (III) chloride).²⁷

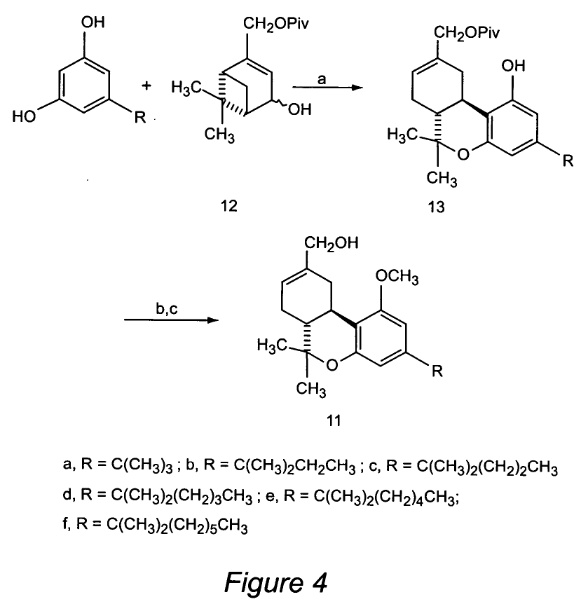

In the 11-hydroxy-3-(1′,1′-dimethylalkyl)-1-methoxy-Δ⁸-THC series, in addition to the dimethylethyl through dimethylhexyl analogues (11a-11e, FIG. 4), 11-hydroxy-3-(1′,1′-dimethylheptyl)-1-methoxy-Δ⁸-THC was prepared. The dimethylheptyl analogues in the methyl ether series (4, FIG. 1) and 1-deoxy-11-hydroxy (1, FIG. 1) series had been prepared previously. ^{19 21} The initial synthetic approach to this series of compounds was based upon a procedure developed by Mechoulam for the synthesis of 11-hydroxy-Δ⁸-THC-DMH (HU-210), and which we had used previously in the synthesis of 11-hydroxy-(10 S, 20 R)-dimethylheptyl-Δ⁸-THC. ^{28,29} In a modification of this protocol, the appropriate resorcinol is condensed with 4-hydroxymyrtenyl pivalate (12, FIG. 4) to provide the 11-pivaloyloxy-Δ⁸-THC (13a and 13b, FIG. 4).Conversion to the methyl ether, followed by reduction with lithium aluminum hydride provides the corresponding 11-hydroxy-3-(1′,1′-dimethylalkyl)-1-methoxy-Δ⁸-THC. This procedure was employed to prepare the first two members of the homologeous series (11a and 11b, FIG. 4), however the overall yields were quite low. The other three members of this series (11c-11e, FIG. 4) were prepared from the corresponding 1-methyl ether (6c-6e, FIG. 2) by selenium dioxide oxidation followed by reduction of the corresponding aldehyde in a procedure analogous to that employed for the synthesis of the 1-deoxy-11-hydroxy compounds. Reduction of the 11-oxo compounds using Luche conditions²⁷ gave excellent yields of the corresponding 11-hydroxy cannabinoids (11c-11e, FIG. 4).

The affinities of 1-methoxy-, 11-hydroxy-and 11-hydroxy-1-methoxy-Δ⁸-THC analogues 6 (FIG. 2), 9 (FIG. 3) and 11 (FIG. 4) for the CB₁ receptor were determined by measuring their ability to displace the potent cannabinoid [³H] CP 55,940 from its binding site in a membrane preparation from rat brain as described by Compton et al.⁸ Affinities for the CB₂ receptor were determined by measuring the ability of the compounds to displace [³H] CP 55,940 from a cloned human receptor preparation using the procedure described by Showalter et al.³⁰ The results of these determinations are summarized in Table 1. Also included in Table 1 are the receptor affinities for cannabinoids 1-4 (FIG. 1), Δ⁸- and Δ⁹-THC.

TABLE 1
Receptor affinities (mean ± SEM) of 1-deoxycannabinoids
and related compounds

| | Kᵢ (nM) | | |
|---|---|---|---|
| Compound | CB₁ | CB₂ | Ratio CB₁/CB₂ |
| Δ⁹-THC | 41 ± 2^{a} | 36 ± 10^{b} | 1.1 |
| Δ⁸-THC | 44 ± 12^{c} | 44 ± 17^{c} | 1.0 |
| 1-Deoxy-11-hydroxy-3-(1′,1′-dimethylheptyl)-Δ⁸- | 1.2 ± 0.1^{d} | 0.03 ± 0.02^{d} | 40 |
| THC (1) | | | |
| 1-Deoxy-3-(1′,1′-dimethylheptyl)-Δ⁸-THC (2) | 22.8 ± 7.3^{d} | 2.9 ± 1.6^{d} | 7.9 |
| 3-(1′,1′-Dimethylheptyl)-1-methoxy-Δ⁸-THC (4) | 713 ± 68 | 57 ± 12 | 12 |
| 3-(1′,1′-Dimethylheptyl)-1-methoxy-Δ⁸-THC (4) | 924 ± 104^{c} | 65 ± 8.2^{c} | 14 |
| 3-(1′,1′-Dimethylbutyl)-1-deoxy-Δ⁸-THC (3) | 677 ± 132^{c} | 3.4 ± 1.0^{c} | 199 |
| 3-(1′,1′-Dimethylethyl)-1-methoxy-Δ⁸-THC (6a) | >10,000 | 1867 ± 867 | 5.4 |
| 3-(1′,1′-Dimethylpropyl)-1-methoxy-Δ⁸-THC | >10,000 | 1404 ± 66 | 7.1 |
| (6b) | | | |
| 3-(1′,1′-Dimethylbutyl)-1-methoxy-Δ⁸-THC (6c) | >10,000 | 325 ± 70 | 31 |
| 3-(1′,1′-Dimethylpentyl)-1-methoxy-Δ⁸-THC | 4001 ± 282 | 43 ± 3 | 93 |
| (6d) | | | |
| 3-(1′,1′-Dimethylhexyl)-1-methoxy-Δ⁸-THC (6e) | 3134 ± 110 | 18 ± 2 | 174 |
| 1-Deoxy-11-hydroxy-3-(1′,1′-dimethylethyl)-Δ⁸- | 270 ± 58 | 18 ± 2 | 15 |
| THC (9a) | | | |
| 1-Deoxy-11-hydroxy-3-(1′,1′-dimethylpropyl)-Δ⁸- | 187 ± 23 | 5.6 ± 1.7 | 33 |
| THC (9b) | | | |
| 1-Deoxy-11-hydroxy-3-(1′,1′-dimethylbutyl)-Δ⁸- | 84 ± 16 | 3.4 ± 0.5 | 25 |
| THC (9c) | | | |
| 1-Deoxy-11-hydroxy-3-(1′,1′-dimethylpentyl)-Δ⁸- | 8.8 ± 1.4 | 1.6 ± 0.03 | 5.5 |
| THC (9d) | | | |
| 1-Deoxy-11-hydroxy-3-(1′,1′-dimethylhexyl)-Δ⁸- | 1.8 ± 0.3 | 0.52 ± 0.03 | 3.5 |
| THC (9e) | | | |
| 11-Hydroxy-3-(1′,1′-dimethylethyl)-1-methoxy- | 1856 ± 148 | 333 ± 104 | 5.6 |
| Δ⁸-THC (11a) | | | |
| 11-Hydroxy-3-(1′,1′-dimethylpropyl)-1-methoxy- | 1008 ± 117 | 85 ± 21 | 12 |
| Δ⁸-THC (11b) | | | |
| 11-Hydroxy-3-(1′,1′-dimethylbutyl)-1-methoxy- | 347 ± 34 | 28 ± 1 | 12 |
| Δ⁸-THC (11c) | | | |
| 11-Hydroxy-3-(1′,1′-dimethylpentyl)-1-methoxy- | 40 ± 6 | 4.4 ± 0.3 | 9.1 |
| Δ⁸-THC (11d) | | | |
| 11-Hydroxy-3-(1′,1′-dimethylhexyl)-1-methoxy- | 15 ± 3 | 1.4 ± 0.1 | 11 |
| Δ⁸-THC (11e) | | | |
| 11-Hydroxy-3-(1′,1′-dimethylheptyl)-1-methoxy- | 14 ± 3 | 1.0 ± 0.3 | 14 |
| Δ⁸-THC (11f) | | | |

^{a}ref 8;
^{b}ref 30;
^{c}ref 20;
^{d}ref 19.

In the 1-methoxy-Δ⁸-THC series (6a-6e, FIG. 2) none of these compounds have appreciable affinity for the CB₁ receptor, with Kᵢ values of 3134 110 nM for the dimethylhexyl analogue (6e) to Kᵢ>10,000 nM for the dimethylethyl through dimethylbutyl compounds (6a-6c).This series of compounds does, however, show considerable selectivity for the CB₂ receptor. There is an incremental increase in CB₂ receptor affinity with Kᵢ=1867±867 for the dimethylbutyl compound (6c),increasing to Kᵢ=18±2 nM for 3-(1′,1′-dimethylhexyl)-1-methoxy-Δ⁸-THC (6e). 3-(1′,1′-Dimethylhexyl)-1-methoxy-Δ⁸-THC (6e) shows nearly 175 fold selectivity for the CB₂ receptor. For 3-(1′,1′-dimethylheptyl)-1-methoxy-Δ⁸-THC (4) we reported Kᵢ=924±104 nM at CB₁ and 65±8 nM at CB₂.²⁰However, for the same compound, Gareau et al. found Kᵢ=15,850±2960 nM at CB₁ and 20±12 nM at CB₂.²¹Ross et al. found somewhat different values, Kᵢ=1043±296 nM at CB₁ and 6.4+2.2 nM at CB₂.²³ In view of these variations in the reported data for 1-methoxy-Δ⁸-THC-DMH (4),the preparation of this compound was repeated, and new binding data for both receptors were obtained. The new data, Kᵢ=713+68 nM at CB₁ and Kᵢ=57+12 nM at CB₂ are essentially the same as those we reported previously.²⁰ The differences in receptor affinity between those we have determined and those determined by other groups may be due to a number of factors, including somewhat different cell lines and slightly different laboratory procedures employed in carrying out the determinations.

As mentioned above, our CB₁ receptor affinities were determined using a rat brain membrane preparation while Gareau et al. employed a human CB₁ receptor preparation which was not described in detail.²¹ The binding assays described by Ross et al. were carried out using CHO (Chinese hamster ovary) cells transfected with human CB₁ and CB₂ receptors.²³ Our CB₂ data were obtained as described in the Experimental using HEK (human embryonic kidney) cells transfected with human CB₂ receptors.

The 1-deoxy-11-hydroxy-Δ⁸-THC analogues (9a-9e, FIG. 3) have from modest to very high affinity for the CB₁ receptor, and show moderate selectivity for the CB₂ receptor. The first member of the homologous series, 1-deoxy-3-(1′,1′-dimethylethyl)-11-hydroxy-Δ⁸-THC (9a) has Kᵢ=270±58 nM at the CB₁ receptor, with Kᵢ=18±2 nM at CB₂ receptor affinity at CB₁ improves to Kᵢ=1.8±0.3 nM and CB₂ affinity increases to Kᵢ=0.52+0.03 nM for the dimethylhexyl analogue (9e).The most selective compound in this series is the dimethylpropyl analogue (9b)which has 25 fold greater affinity for the CB₂ receptor (Kᵢ=187±23 nM at CB₁ and Kᵢ=5.6±1.7 nM at CB₂).

The compounds in the 11-hydroxy-3-(1′,1′-dimethylalkyl)-1-methoxy-Δ⁸-THC series (11a-11f, FIG. 4) also show moderate selectivity for the CB₂ receptor, but the CB₁ receptor affinities increase significantly in the higher members of this homologous series. For 11-Hydroxy-3-(1′,1′-dimethylethyl)-1-methoxy-Δ⁸-THC (11a), Kᵢ=1856±148 nM at CB₁ and Kᵢ=333±104 at CB₂. Affinity for both receptors improves to Kᵢ=15±3 nM at CB₁ for 11-hydroxy-3-(1′,1′-dimethylhexyl)-1-methoxy-Δ⁸-THC (11e) with Kᵢ=1.4±0.3 nM at CB₂. The receptor affinities for 11-hydroxy-3-(1′,1′-dimethylheptyl)-1-methoxy-Δ⁸-THC (11f)are essentially identical to those for the dimethylhexyl analogue, with Kᵢ=14±3 nM at CB₁ and 1.0 ±0.3 at CB₂.

The data summarized in Table 1, are in general agreement with the preliminary SAR for the CB₂ receptor which we developed based upon our study of 1-deox-Δ⁸-THC analogues.²⁰ In the 1′,1′-dimethyl-1-deoxy-Δ⁸-THC series described previously, those compounds with a three to seven carbon side chain (2 and 8b-8e) all have high affinity for the CB₂ receptor (Kᵢ<20 nM).Of the three new series of CB₂ selective cannabinoid receptor ligands, only the 1-deoxy-11-hydroxy-Δ⁸-THC analogues (1 and 9a-9e) show uniformly high affinity for the CB₂ receptor, with Kᵢ=0.032±0.019 nM for the dimethyl-heptyl analogue (1)¹⁹ to Kᵢ=18.1±1.8 nM for the lowest member of the homologous series (9a). There is a progressive improvement in CB₂ receptor affinity as the length of the side chain increases from two to seven carbon atoms. These compounds also show from modest to high affinity for the CB₁ receptor, increasing from Kᵢ=270±58 nM for the dimethylethyl analogue (9a) to Kᵢ=1.2±0.1 nM for the dimethylheptyl compound (1) reported previously.¹⁹ The relatively high CB₁ receptor affinities for the compounds in this series may be attributed to the 11-hydroxyl group serving as a surrogate for the phenolic hydroxyl in more traditional cannabinoids as suggested by molecular modeling studies carried out on 1, combined with a 3-(1′,1′-dimethyl-alkyl) substituent of sufficient length to interact with the lipophilic portion of the receptor.¹⁹

The compounds in the 1-methoxy series (4 and 6a to 6e) all have little affinity for the CB₁ receptor, with CB₂ affinities ranging from very slight for the dimethylethyl analogue (6a, Kᵢ=1867±867 nM) to quite high for the dimethylhexyl compound (6e, Kᵢ=18±2 nM). As reported previously, the dimethylheptyl analogue (4) has little affinity for the CB₁ receptor and moderate affinity for the CB₂ receptor. The dimethylhexyl methyl ether (6e) is a highly selective CB₂ receptor ligand with good affinity for the CB₂ receptor and very little affinity for the CB₁ receptor.

The compounds of the 11-hydroxy-1-methoxy series (11a-11f) are intermediate between those of the other two series of ligands in their affinities for both receptors. The lower members of this series (11a-11c) have little affinity for the CB₁ receptor with Kᵢ=1856±148 nM for the dimethylethyl analogue (11a) and Kᵢ=347±34 mM for the dimethylbutyl compound (11c). The higher members of this series have from moderate affinity (11d, Kᵢ=40±6 nM) for the CB₂ receptor to high affinity for the dimethylhexyl (11e) and dimethylheptyl (11f) analogues. The affinities of 11e and 11f are identical within experimental error for each receptor, with Kᵢ=14 nM at CB₁ and 1.2 nM at CB₂.

In terms of the SAR for 1-methoxy-, 1-deoxy-11-hydroxy and 11-hydroxy-1-methoxy-Δ⁸-THC analogues, it is apparent that an 11-hydroxyl substituent enhances affinity for both the CB₁ and CB₂ receptors. Also, in the 3-(1,1-dimethylalkyl) series the length of the side chain plays a critical role in determining affinity for both receptors. It is somewhat important for CB₂ affinity, particularly in the methyl ether series (6),but for significant CB₁ affinity a chain length of at least five carbon atoms is essential.

In summary, although several of these compounds show selectivity for the CB₂ receptor, only five of them, 1-methoxy cannabinoids 6d and 6e, 1-deoxy-11-hydroxy compounds 9a and 9b, and 11-hydroxy-3-(1′,1′-dimethylbutyl)-1-methoxy-Δ⁸-THC (11c) have a combination of high affinity for the CB₂ receptor and little affinity for the CB₁receptor. Only 3-(1′,1′-dimethyl-hexyl)-1-methoxy-Δ⁸-THC (6e, JWH-229) with Kᵢ=3134±110 nM at CB₁ and Kᵢ=18±2 nM at CB₂ is comparable in selectivity to 1-deoxy-3-(1′,1′-dimethylhexyl)-Δ⁸-THC (3, JWH-133) with Kᵢ=677±132 nM at CB₁ and Kᵢ=3.4±1.0 nM at CB₂.²⁰ Although JWH-229 (6e) has slightly less affinity for the CB₂ receptor than JWH-133 (3), it has significantly lower affinity for CB₁, and is thus a potentially useful CB₂ selective cannabinoid ligand with very little affinity for CB₁.

Experimental

General

IR spectra were obtained using Nicolet 5DX or Magna spectrometers; ¹H and ¹³C NMR spectra were recorded on a Bruker 300AC spectrometer. Mass spectral analyses were performed on a Hewlett-Packard 5890A capillary gas chromatograph equipped with a mass sensitive detector. HRMS data were obtained in the Mass Spectrometry Laboratory, School of Chemical Sciences, University of Illinois. Ether and THF were distilled from Na-benzophenone ketyl immediately before use, and other solvents were purified using standard procedures. Column chromatography was carried out on Sorbent Technologies silica gel (32-63 μm) using the indicated solvents as eluents. All new compounds were homogeneous to TLC and ¹³C NMR. All target compounds were homogeneous to GLC or TLC in two different solvent systems. TLC was carried out using 200 m silica gel plates using the indicated solvents. GLC analyses were performed on the Hewlett-Packard 5890A GC/MS using a 60 m carbowax column and helium gas as a carrier. An initial column temperature of 60° C. was employed and the temperature was increased at a rate of 1.5 C/min to a maximum temperature of 300° C. with a total run time of 20 min. Elemental analyses were performed by Atlantic Microlab, Norcross, Ga.

1-Methoxy-3-(1′,1′-dimethylpropyl)-Δ⁸-THC (6b).To a solution of 0.569 g (1.8 mmol) of 3-(1′,1′-dimethylpropyl)-Δ⁸-THC (7b)²⁰ in 14 mL of dry DMF under N₂ was added 0.151 g (2.7 mmol) of KOH and 0.33 mL (5.4 mmol)of methyl iodide. The reaction mixture was stirred for 48 h at ambient temperature before being quenched by the addition of 2 mL of aqueous NH₄Cl and removal of the DMF in vacuo. The residue was extracted with three portions of ether and the combined organic extracts were dried (MgSO₄) and concentrated in vacuo. The crude product was initially purified by dry flash chromatography (petroleum ether:ether, 97:3) followed by gradient elution chromatography (petroleum ether:dichloromethane, 9:1 to 8:1)to afford 0.309 g (52%)of 6b as a colorless oil. Further chromatography (petroleum ether:dichloromethane, 85:15) of 0.104 g of this material gave 0.100 g of pure 6b, R_{f} 0.46 (petroleum ether:dichloromethane, 85:15); ¹H NMR (500 MHz, CDCl₃) δ 0.79 (t, J=7.1 Hz,3H),1.09 (s,3H),1.24 (s, 6H),1.38 (s,3H),1.54.1.60 (m,2H),1.70 (s,3H),1.74. 1.81 (m,3H),2.12.2.14 (m,1H),2.67 (td, J=4.7, 11.1 Hz,1H),3.15 (dd, J=4.7, 17.1 Hz,1H),3.81 (s, 3H),5.42 (br.s,1H),6.38 (d, J=1.6 Hz,1H),6.42(d, J=1.6 Hz,1H); ¹³C NMR (125.8 MHz,CDCl₃) δ 14.1, 18.4, 23.4, 27.8, 27.9, 28.7, 31.7, 36.3, 37.6, 44.3, 45.0, 55.1, 76.4, 100.7, 108.3, 111.2, 119.2, 135.0, 149.8, 153.9, 158.6; MS (ED) m/z 328 (65), 299 (45), 245 (100); [αD²⁰ −210° (c=0.29,CH₂Cl₂) HRMS calcd for C₂₂H₃₂O₂: 328.2404, found 328.2402.

1-Methoxy-3-(1′,1′-dimethylethyl)-Δ⁸-THC (6a). Methoxy cannabinoid 6a was prepared by the procedure described above for the preparation of 6b. Methylation of 0.384 g (1.28 mmol) of 7a²⁰ gave 0.173 g (43%)of 6a as a colorless oil: ¹H NMR (300 MHz, CDCl₃) δ 1.10 (s,3H), 1.29 (s,9H), 1.38 (s,3H), 1.74 (s,3H), 1.76.1.82 (m,3H), 2.11.2.14 (m,1H),2.65 (td, J=4.5, 10.9 Hz, 1H), 3.15 (dd, J=3.8, 17.3 Hz, 1H), 3.81 (s,3H), 5.41 (d, J=4.6 Hz,1H),6.44 (d, J=1.6 Hz, 1H), 6.48 (d, J=1.6 Hz, 1H); ¹³C NMR (75.5 MHz,CDCl₃) δ 18.5, 23 (5), 27.6, 28.0, 31.2, 31.7, 34.7, 36.1, 45.0, 55.1, 76.5, 100.2, 107.6, 111.7, 119.2, 135.0, 150.9, 154.0, 158.7; MS (EI) m/z 314 (52), 299 (8), 246 (13), 231 (100);[αD²⁰ −206° (c=2.65,CH₂Cl₂); HRMS calcd for C₂₁H₃₀O₂: 314.2245, found 314.2246.

11-Methoxy-3-(1′,1′-dimethylbutyl)-Δ⁸-THC (6c). Methoxy cannabinoid 6c was prepared by the procedure described above for the preparation of 6b. Methylation of 0.782g (2.38 mmol)of 7c²⁰ gave 0.461 g (43%)of 6c as a colorless oil: ¹H NMR (500 MHz,CDCl₃) δ 0.82(t, J=7.4 Hz, 3H), 1.09 (s, 3H), 1.05.1.15 (m, 2H), 1.25 (s, 6H),1.38 (s, 3H), 1.49.1.55 (m, 2H),1.70 (s, 3H), 1.74.1.85 (m, 3H), 2.09.2.16 (m, 1H), 2.66 (td, J=4.6, 11.0 Hz, 1H), 3.16 (dd, J=3.2, 16.5 Hz, 1H), 3.78 (s,3H), 5.42 (d,4.1H), 6.38 (d, J=0.9 Hz, 1H), 6.43 (d, J=0.9 Hz, 1H); ¹³C NMR (125.8 MHz,CDCl₃) δ 14.9, 18.1, 18.5, 23.7, 27.7, 28.1, 28.9, 29.0, 31.8, 36.3, 37.8, 45.2, 47.2, 55.2, 76.9, 100.9, 108.3, 111.7, 119.4, 135.1, 149.7, 154.1, 158.8; MS (EI) m/z 342(37), 300 (100), 286 (20),259 (38); [αD²⁰ −258° (c=0.79,CHCl₃); HRMS calcd for C₂₃H₃₄O₂: 342.2564, found 342.2559.

1-Methoxy-3-(1′,1′-dimethylpentyl)-Δ⁸-THC (6d). Methoxy cannabinoid 6d was prepared by the procedure described above for the preparation of 6b. Methylation of 2.80 g (8.17 mmol) of 7d²⁰ gave 2.83 g (97%)of 6d as a colorless oil: ¹H NMR (300 MHz,CDCl₃) δ 0.83 (t, J=7.1 Hz, 3H), 1.03.1.11 (m, 2H), 1.10 (s, 3H), 1.16.1.29 (m,2H), 1.25 (s,6H), 1.38 (s,3H), 1.51.1.60 (m, 2H), 1.70 (s,3H), 1.73.1.90 (m,3H), 2.05.2.12 (m,1H), 2.66 (td, J=4.7, 10.8 Hz,1H), 3.06 (dd, J=4.0 Hz,1H), 3.81 (s,3H),5.41 (d, J=4.8 Hz,1H), 6.38 (d, J=1.6 Hz, 1H),6.43 (d, J=1.6 Hz,1H); ¹³C NMR (75.5 MHz, CDCl₃) δ 14.1,18.4, 23.4,23.5, 26.9, 27.6, 27.9, 28.8, 31.7, 36.2, 37.6, 44.3, 45.0, 55.1, 76.5, 100.7, 108.2, 111.6, 119.2, 135.0, 149.7, 153.9, 158.7; MS (EI) m/z 356 (45), 300 (100), 286 (25), 273 (30); HRMS calcd for C₂₄H₃₆O₂: 356.2716, found 356.2715; [αD²⁰ −234° (c=0.24,CHCl₃).

1-Methoxy-3-(1′,1′-dimethylhexyl)-Δ⁸-THC (6e). Methoxy cannabinoid 6e was prepared by the procedure described above for the preparation of 6b. Methylation of 2.59 g (7.26 mmol)of 7e²⁰ gave 2.54 g (94%) of 6e as a colorless oil: ¹H NMR (300 MHz,CDCl₃) δ 0.83 (t, J=7.1 Hz, 3H), 1.01.1.30 (m,6H), 1.10 (s, 3H), 1.24 (s, 6H), 1.38 (s, 3H),1.48.1.56 (m,2H), 1.65.1.90 (m, 3H), 1.70 (s,3H), 2.11.2.18 (m,1H), 2.67 (td, J=4.7, 10.9 Hz, 1H), 3.15 (dd, J=4.1, 17.0 Hz, 1H), 3.81 (s, 3H), 5.41 (s, 1H), 6.38 (d, J=1.6 Hz, 1H), 6.42(d, J=1.6 Hz, 1H); ¹³C NMR (75.5 MHz, CDCl₃) δ 14.1, 18.4, 22.5, 23.5, 24.3, 27.6, 27.7, 28.0, 28.8, 31.7, 32.6, 36.2, 37.7, 44.5, 45.1, 55.1, 76.4, 100.7, 108.3, 111.6, 119.2, 135.1, 149.7, 153.9, 158.7; MS (EI) m/z 370 (40), 300 (100), 287 (35); [αD²⁰ −219° (c=0.21,CHCl₃); HRMS calcd for C₂₅H₃₈O₂: 370.2871, found 370.2872.

1-Deoxy-3-(1′,1′-dimethylpropyl)-11-hydroxy-Δ⁸-THC (9b).To a stirred suspension of 1.07 g (3.58 mmol) of 1-deoxycannabinoid 8b²⁰ in 16 mL of ethanol at ambient temperature was added dropwise over 30 min a solution of 0.96 g of SeO₂ (8.73 mmol) in 17.6 mL of ethanol/water (10:1). The reaction mixture was heated at reflux for 18 h, filtered through a pad of Celite, which was subsequently washed with three portions of methanol, and the combined organic extracts were concentrated in vacuo. The residue was extracted with three portions of ether and the resulting ethereal solution was washed successively with water then saturated NaHCO₃.The organic phase was dried (MgSO₄)and concentrated in vacuo to afford crude aldehyde 10b which was purified by dry flash chromatography (ethyl acetate:petroleum ether, 9:1) to afford 0.96 g of 10b as a light brown oil which was used without further purification. To a solution of 0.96 g (3.10 mmol) of the crude aldehyde in 35 mL of dry THF at 0° C. under N₂was added 0.12 g (3.07 mmol) of LiAlH₄.The reaction mixture was allowed to warm to room temperature, stirred for 2 h and then quenched with aqueous NH₄Cl. After filtering through a pad of Celite, which was subsequently washed with diethyl ether, the combined organic extracts were dried (MgSO₄)and concentrated in vacuo to afford the crude product. Initial chromatography (gradient elution with 17% diethyl ether to 35% diethyl ether in petroleum ether) gave a pale yellow resin which was further purified by chromatography (gradient elution with 5% acetone to 7% acetone in petroleum ether) to afford 0.31 g (32% for two steps)of 9b as a white foam: ¹H NMR (500 MHz,CDCl₃) δ 0.69 (t, J=7.8 Hz,3H), 1.17 (s,3H), 1.24 (s,6H), 1.40 (s,3H), 1.60 (q, J=7.4 Hz, 1H), 1.72.2.02 (m, 5H), 2.20.2.28 (m,1H), 2.69 (td, J=5.5, 11.0 Hz,1H), 2.80 (dd, J=4.2, 16.5 Hz,1H), 4.05 (d, J=12.8 Hz,1H), 4.08 (d, J=12.8 Hz, 1H), 5.76 (br.s, 1H), 6.76 (d, J=1.8 Hz, 1H), 6.85 (dd, J=1.8, 7.8 Hz, 1H), 7.14 (dd, J=0.9, 8.2 Hz, 1H); ¹³C NMR (125.8 MHz,CDCl₃) δ 9.34, 19.2, 27.3, 27.8, 28.5, 31.9, 32.1, 36.9 ,37.7, 43.2, 67.0, 76.9, 114.9, 118.0, 121.7, 122.2, 126.2, 137.1, 149.4, 152.6; MS (EI) m/z 314 (59), 312 (20), 299 (13), 285 (100), 207 (100); [αD²⁰ −126° (c=9.0,CHCl₃); HRMS calcd for C₂₁H₃₀O₂: 314.2251, found 314.2246.

1-Deoxy-3-(1′,1′-dimethylethyl)-11-hydroxy-Δ⁸-THC (9a). 11-Hydroxycannabinoid 9a was prepared by the procedure described above for the preparation of 9b. Stepwise oxidation and reduction of 1.27 g (4.46 mmol) of 8a²⁰ gave 0.35 g (26% for two steps) of hydroxy cannabinoid 9a as a white foam: ¹H NMR (500 MHz, CDCl₃) δ 1.17 (s,3H), 1.29 (s,9H), 1.41 (s,3H), 1.76 dt, J=5.8, 13.0 Hz, 1H), 1.80 .1.90 (m, 1H), 1.94 (br. t, J=13.0 Hz, 1H), 2.08.2.28 (m,2H), 2.69 (dt, J=5.8, 3.1 Hz,1H), 2.80 (dd, J=5.3, 17.5 Hz,1H), 4.04 (d, J=17.0 Hz,1H), 4.08 (d, J=17.0 Hz,1H), 5.77 (br. s, 1H), 6.84 (d, J=1.4 Hz, 1H),6.92(dd, J=1.8, 8.2 Hz, 1H),7.17 (d, J=8.2 Hz, 1H); ¹³C NMR (125.8 MHz, CDCl₃) δ 19.3, 27.3, 27.8, 31.4, 31.9, 32.1, 34.4, 43.2, 66.9, 77.0, 114.2, 117.4, 121.7, 126.4, 122.4, 137.1, 150.9, 152.6; MS (EI) m/z 300 (100), 298 (42), 285 (36), 207 (51); [αD²⁰ −103° (c=10.4,CHCl₃); HRMS calcd for C₂₀H₂₈O₂: 300.2090, found 300.2089.

1-Deoxy-3-(1′,1′-dimethylbutyl)-11-hydroxy-Δ⁸-THC (9c). 11-Hydroxycannabinoid 9c was prepared by the procedure described above for the preparation of 9b. Stepwise oxidation and reduction of 1.20 g (4.09 mmol) of 8c 20 gave 0.33 g (25% for two steps)of hydroxy cannabinoid 9c as a white foam: ¹H NMR (500 MHz, CDCl₃) δ 0.81 (t, J=7.4 Hz,3H),1.03 .1.14 (m, 2H), 1.16 (s, 3H),1.25(s, 6H),1.40 (s, 3H), 1.49 .1.56 (m, 2H), 1.74 (td, J=5.0, 11.9 Hz, 1H), 1.82 .1.90 (m, 1H), 1.91 .2.02 (m, 2H), 2.23 (br.d, J=17.8 Hz,1H), 2.68 (td, J=5.5, 11.0 Hz,1H), 2.80 (dd, J=5.0, 17.0 Hz,1H), 4.04 (d, J=12.8 Hz,1H), 4.08 (d, J=12.8 Hz,1H), 5.75 (br.s,1H), 6.76 (d, J=1.8 Hz, 1H), 6.84 (dd, J=1.8, 8.2 Hz,1H),7.13 (d, J=7.8 Hz,1H); ³C NMR (75.5 MHz,CDCl₃) δ 14.7, 17.9,19.1, 27.1, 27.6, 28.7, 28.8, 31.8, 32.0, 37.4, 43.0, 46.9, 66.8,7 6.6, 114.6, 117.7, 121.5, 122.0, 126.0, 136.9, 149.5, 152.4; MS (EI) m/z 328 (46), 285 (100), 207 (60); [αD²⁰ −153°=10.3,CHCl₃); HRMS calcd for C₂₂H₃₂O₂: 328.2402, found 328.2402.

1-Deoxy-3-(1′,1′-dimethylpentyl)-11-hydroxy-Δ⁸-THC (9d). Hydroxycannabinoid 9d was prepared by the procedure described above for the preparation of 9b. Stepwise oxidation and reduction of 1.60 g (4.90 mmol)of 8d 20 gave 0.48 g (28% for two steps) of hydroxy cannabinoid 9d as a white foam: ¹H NMR (500 MHz,CDCl₃) δ 0.81 (t, J=7.3 Hz, 3H), 1.01 .1.09 (m, 2H), 1.17 (s, 3H), 1.17.1.23 (m,2H), 1.24 (s, 6H), 1.41 (s, 3H), 1.51 .1.57 (m, 2H), 1.76 (td, J=4.6, 11.4 Hz, 1H), 1.82.1.91 (m, 1H), 1.95 .2.03 (m, 2H), 2.25 (dt, J=8, 17.9 Hz, 1H), 2.69 (dt, J=5.5, 11.4 Hz, 1H), 2.80 (dd, J=4.6, 16.5 Hz, 1H), 4.06 (d, J=12.8 Hz,1H), 4.09 (d, J=12.8 Hz, 1H), 5.76 (br.s, 1H), 6.76 (d, J=1.8 Hz,1H), 6.84 (dd, J=1.8, 7.8 Hz,1H),7.14 (d, J=8.2 Hz, 1H); ¹³C NMR (125.8 MHz,CDCl₃) δ 14.2, 19.2, 23.5, 27.0, 27.3, 27.8, 29.0, 31.9, 32.1, 37.4,43.1,44.4,67.1, 76.9, 114.8, 117.9, 121.8, 122.2, 126.2, 137.1, 149.8, 152.6; MS (EI) m/z 342 (33), 285 (100,), 269 (29), 255 (19); [αD²⁰ +63° (c=14.3, CHCl₃); HRMS calcd for C₂₃H₃₄O₂: 342.2562, found 342.2559.

1-Deoxy-3-(1′,1′-dimethylhexyl)-11-hydroxy-Δ⁸-THC (9e). 11-Hydroxycannabinoid 9e was prepared by the procedure described above for the preparation of 9b. Stepwise oxidation and reduction of 1.72 g (5.05 mmol) of 8e²¹gave 0.231 g (13% for two steps)of hydroxy cannabinoid 9e as a white foam: ¹H NMR (500 MHz, CDCl₃) δ 0.82(t, J=6.8 Hz, 3H), 1.03 .1.12 (m,2H), 1.15 .1.24 (m, 4H), 1.16 (s, 3H), 1.24 (s, 6H), 1.40 (s, 3H), 1.51 .1.57 (m, 2H), 1.76 (td, J=4.6, 11.9 Hz,1H), 1.81 .2.03 (m, 3H), 2.23 (dt, J=8.0, 16.5 Hz, 1H), 2.69 (td, J=5.5, 11.0 Hz, 1H), 2.79 (dd, J=5.0, 17.0 Hz, 1H), 4.04 (d, J=12.8 Hz, 1H), 4.08 (d, J=12.8 Hz, 1H), 5.75 (br.s, 1H), 6.76 (d, J=2.3 Hz, 1H), 6.84 (dd, J=1.8, 8.2 Hz,1H),7.13 (d, J=8.2 Hz,1H); ¹³C NMR (125.8 MHz,CDCl₃) δ 14.2, 19.2, 22.720 , 24.5, 27.3, 27.8, 28.9, 29.0, 31.9, 32.1, 32.7, 37.5, 43.2, 44.6, 67.0, 77.2, 114.8, 117.9, 121.7, 122.2, 126.2, 137.1, 149.8, 152.6; MS (EI) m/z 356 (32), 285 (100), 269 (18); [αD²⁰ 144 (c=6.5,CHCl₃); HRMS calcd for C₂₄H₃₆O₂: 356.2715, found 356.2715.

3-(1′,1′-Dimethylpropyl)-11-pivaloyloxy-Δ⁸-THC (13b). To a solution of 0.507 g (2.81 mmol) of crude 2-methyl-2-(3,5-dihydroxyphenyl) propane and 0.709 g (2.81 mmol) of 4-hydroxymyrtenyl pivalate (12) in 188 mL of dry dichloromethane at −20° C. was added dropwise with stirring 1.94 mL (14.1 mmol) of boron trifluoride etherate. The mixture was allowed to warm to 0° C. and stirred for 2 h, poured onto ice and neutralized with saturated aqueous NaHCO₃. After extraction with ether, the combined organic extracts were dried (MgSO₄) and concentrated in vacuo to afford the crude product as a dark brown foam. Chromatography (petroleum ether:ethyl acetate, 95:5) and subsequent recrystallization (heptane:ethyl acetate, 95:5)afforded 0.233 g (20%)of pure 13b as white crystals, mp 214-215° C.: ¹H NMR (300 MHz,CDCl₃) δ 0.67 (t, J=7.4 Hz, 3H), 1.10 (s, 3H),1.18 (s,6H),1.20 (s,9H),1.38 (s,3H),1.56 (q, J=7.3 Hz,2H), 1.80.1.91 (m, 3H), 2.21 .2.29 (m, 1H), 2.71 (td, J=4.6, 11.1 Hz, 1H), 3.36 (dd, J=3.9, 16.8 Hz, 1H), 4.48 (s, 2H), 5.20 (br.s, 1H), 5.73 (d, J=4.9 Hz, 1H),6.21 (d, J=1.8 Hz, 1H), 6.37 (d, J=1.7 Hz, 1H); ¹³C NMR (75.5 MHz,CDCl₃) δ 9.2, 18.4, 27.2, 28.2, 31.2, 31.6, 36.8, 38.9, 44.8, 68.1, 76.5, 105.6, 107.8, 109.7, 123.2, 133.9, 149.7, 154.3, 154.7, 178.8; anal. calcd for C₂₆H₃₈O₄: C,75.33; H,9.24; found:C,75.15; H,9.34.

1-Methoxy-3-(1′,1′-dimethylpropyl)-11-pivaloyloxy-Δ⁸-THC. Methylation of 0.556 g (1.34 mmol)of 13b by the procedure used for the preparation of 6b gave 0.454 g (79%) of the corresponding methyl ether as a colorless oil following chromatography (petroleum ether:ether, 97.5:2.5 to 95:5): ¹H NMR (300 MHz,CDCl₃) δ 0.70 (t, J=7.4 Hz, 3H), 1.10 (s, 3H), 1.24 (s, 15H), 1.39 (s, 3H), 1.57 (q, J=7.7 Hz, 2H),1.70 .1.92 (m, 3H), 2.21 .2.29 (m,1H), 2.67 (td, J=4.6, 11.1 Hz,1H), 3.30 (dd, J=3.9, 16.8 Hz,1H), 3.79 (s,3H), 4.49 (s, 2H), 5.76 (br.s,1H), 6.38 (d, J=1.5 Hz,1H), 6.43 (d, J=1.6 Hz,1H).

1-Methoxy-3-(1′,1′-dimethylpropyl)-11-hydroxy-Δ⁸-THC (11b). To a solution of 0.454 g (1.06 mmol)of pivalate ester in 33 mL of dry THF under N₂ at 0° C. was added 0.051 g (1.34 mmol)of LiAlH₄.The mixture was allowed to warn to room temperature, stirred for 1 h and quenched with 15 mL of aqueous NH₄Cl. The solids were filtered off through a pad of Celite, which was subsequently washed with ether. The combined organic fractions were dried (MgSO₄) and concentrated in vacuo to afford the crude product. Chromatography (gradient elution, petroleum ether:acetone, 95:5 to 93:7) gave 0.233 g (68%)of pure 11b as a colorless resin: ¹H NMR (500 MHz,CDCl₃) δ 0.69 (t, J=7.3 Hz, 3H), 1.11 (s,3H), 1.25 (s,6H), 1.39 (s, 3H), 1.59 (q, J=7.4 Hz, 2H),1.78 .1.91 (m, 3H), 2.18 .2.26 (m, 1H), 2.67 (td, J=4.6, 11.0 Hz,1H), 3.31 (dd, J=4.6, 16.5 Hz, 1H), 3.80 (s, 3H), 4.03 (d, J=13.3 Hz, 1H), 4.06 (d, J=13.3 Hz, 1H), 5.73 (d, J=4.6 Hz, 1H), 6.38 (d, J=1.6 Hz, 1H), 6.43 (d, J=1.6 Hz, 1H); ¹³C NMR (125.8 MHz,CDCl₃) δ 9.3, 18.5, 27.6, 27.7, 28.4, 31.6, 31.9, 36.9, 38.0, 45.3, 55.2, 67.2, 76.6, 100.8, 108.4, 111.3, 120.8, 138.6, 149.5, 154.0,158.7, 158.7; MS (EI) m/z 344(29), 315(24), 281 (28), 245 (64), 207 (100); [αD²⁰ −209° (c=0.46,CH₂Cl₂); HRMS calcd for C₂₂H₃₂O₃ 344.2351, found 344.2351.

3-(1′,1′-Dimethylethyl)-11-pivaloyloxy-Δ⁸-THC (13a). 11-Pivaloyloxycannabinoid 13a was prepared by the procedure described above for the preparation of 13b. From 1.62 g (9.76 mmol) of resorcinol 0.892 g (23%)of 13a was obtained as a colorless oil following chromatography (petroleum ether/ether, 97.5:2.5 to 95:5): ¹H NMR (500 MHz,CDCl₃) δ 1.05 (s, 3H), 1.15 (s, 9H), 1.16 (s, 9H), 1.32 (s, 3H), 1.73 .1.82 (m, 3H), 2.14.2.20 (m,1H), 2.63 (td, J=4.6, 11.0 Hz, 1H),3.30 (dd, J=4.2, 11.9 Hz, 1H), 4.43 (br.s, 2H), 5.35 (br. s, 1H), 5.68 (d, J=4.6 Hz, 1H), 6.24 (d, J=1.8 Hz, 1H), 6.36 (d, J=1.8 Hz, 1H); ¹³C NMR (125.8 MHz,CDCl₃) δ 18.6, 27.3, 27.6, 27.7, 31.3, 31.4, 34.4, 39.0, 44.8, 68.3, 76.9, 105.1, 107.2, 109.9, 123.3, 133.9, 151.4, 154.5, 154.9, 178.9, 154.9.

1-Methoxy-3-(1′,1′-dimethylethyl)-11-pivaloyloxy-Δ⁸-THC. Methylation of 0.523 g (1.31 mmol)of 13a by the procedure used for the preparation of 6b gave 0.447 g (82%) of the corresponding methyl ether as a colorless oil following chromatography (petroleum ether/ether, 97.5:2.5 to 95:5): ¹H NMR (300 MHz,CDCl₃) δ 1.11 (s, 3H), 1.23 (s, 9H), 1.29 (s, 9H), 1.39 (s, 3H), 1.74.1.87 (m, 3H), 2.19.2.30 (m,1H), 2.67 (td, J=4.5, 10.9 Hz, 1H), 3.31 (dd, J=3,9,16.9 Hz, 1H), 3.80 (s,3H), 4.49 (br s,2H), 5.75 (d, J=4.4 Hz, 1H), 6.44 (d, J=1.6 Hz, 1H), 6.49 (d, J=1.6 Hz, 1H); ¹³C NMR (75.5 MHz, CDCl₃) δ 18.4, 27.2, 27.5, 27.7, 31.2, 31.4, 31.8, 39.0, 44.9, 55.0, 68.1, 76.3, 100.2, 107.6, 111.2, 123.4, 134.1, 149.9, 154.0, 158.7, 178.4.

1-Methoxy-3-(1′,1′-dimethylethyl)-11-hydroxy-Δ⁸-THC (11a). Reduction of 0.447 g (1.08 mmol)of the pivalate ester by the procedure used for the preparation of 11b gave 0.176 g (49%) of hydroxy cannabinoid 11a as a colorless resin: ¹H NMR (500 MHz,CDCl₃) δ 1.10 (s, 3H), 1.27 (s, 9H), 1.38 (s, 3H), 1.76.1.90 (m, 3H), 2.17.2.24 (m,1H), 2.65 (td, J=4.6, 11.0 Hz,1H), 3.30 (dd, J=4.6, 16.5 Hz, 1H), 3.80 (s, 3H),4.02(d, J=13.3 Hz,1H),4.05 (d, J=13.3 Hz,1H), 5.72(d, J=5.0 Hz,1H), 6.43 (d, J=1.9 Hz, 1H), 6.48 (d, J=1.8 Hz, 1H); ¹³C NMR (125.8 MHz,CDCl₃) δ 18.5, 27.7, 31.3, 31.5, 31.8, 34.8, 45.2, 55.2, 67.2, 76.6, 100.2, 107.6, 111.4, 120.8, 138.6, 151.1, 154.0, 158.8; MS (EI) m/z 330 (34), 299 (14), 231 (100), 207 (92); [αD²⁰ −225° (c=1.08,CH₂Cl₂); HRMS calcd for C₂₁H₃₀O₃: 330.2194, found 330.2195.

3-(1′,1′-Dimethylbutyl)-1-methoxy-11-oxo-Δ⁸-THC (14c). Selenium dioxide oxidation of 0.290 g (0.847 mmol)of 6c, using the procedure described above for the preparation of 10b gave, after chromatography (petroleum ether/ethyl acetate, 9:1), 0.196 g (65%)of aldehyde as a yellow solid:mp 109-110° C.; ¹H NMR (300 MHz,CDCl₃) δ 0.82(t, J=7.2 Hz,3H),1.03 .1.13 (m,2H), 1.10 (s,3H),1.25 (s,6H),1.43 (s,3H),1.50 .1.55 (m, 2H),1.79 .1.93 (m.2),2.04 .2.15 (m,1H),2.51 .2.56 (m, 1H),2.61 (td, J=4.5, 11.2 Hz,1H),3.73 (dd, J=2.1, 17.9 Hz, 1H), 3.82, (s,3H), 6.39 (d, J=1.6 Hz,1H),6.42 (d, J=1.6 Hz,1H), 6.83 (s,1H), 9.50 (s,1H); ³C NMR (75.5 MHz,CDCl₃) δ 14.8, 17.9, 18.2, 27.5, 28.7, 29.2, 30.8, 37.6, 44.9, 47.0, 55.1, 75.8, 100.7, 108.2, 110.2, 142.2, 148.9, 150.1, 153.7, 158.6, 193.7; MS (EI) m/z 356 (55), 306 (100).

1-Methoxy-3-(1′,1′-dimethylbutyl)-11-hydroxy-Δ⁸-THC (11c). To a suspension of 0.196 g (0.551 mmol)of aldehyde 14c in 3.5 mL of dry methanol was added sequentially 0.205 g (0.551 mmol)of CeCl₃.7H₂O and 0.021 g (0.551 mmol)of NaBH₄.The reaction mixture was stirred at ambient temperature for 2 h, the pH was adjusted to 7.0 by the addition of 1M aqueous HCl. After pouring into water the mixture was extracted with three portions of CH₂Cl₂,dried (MgSO₄)and the solvent was removed in vacuo. The residue was purified by chromatography (petroleum ether:ethyl acetate, 4:1) to give 0.185 g (94%) of cannabinoid 11c as a pale yellow gum: ¹H NMR (500 MHz,CDCl₃) δ 0.82 (t, J=7.1 Hz,3H), 1.05.1.14 (m,2H),1.11 (s,3H),1.25 (s,6H),1.39 (s, 3H),1.50 .1.54 (m,2H),1.79 .1.92 (m,3H),2.17 .2.24 (m,1H),2.67 (td, J=4.6, 11.0 Hz,1H),3.31 (dd, J=4.6, 16.5 Hz,1H),3.80 (s,3H),4.05 (s,2H),5.73 (s,1H), 6.38 (d,1.6.1H),6.43 (d, J=1.6 Hz,1H); ¹³C NMR (125.8 MHz,CDCl₃) δ 14.7,17.9,18.3,27.5,27.6,28.7, 28.8, 31.4, 31.8, 37.6, 45.1, 46.9, 55.0, 66.8, 76.2, 100.5, 108.1,111.1,120.4,138.4,149.6,153.8,158.5; [αD²⁰ −238° (c=0.50,CHCl₃); HRMS calcd for C₂₃H₃₄O₃:358.2508, found 358.2509.

1-Methoxy-3-(1′,1′-dimethylpentyl)-11-oxo-Δ⁸-THC (14d). Selenium dioxide oxidation of 2.00 g (5.17 mmol) of 6d, using the procedure described above for the preparation of 10b, gave, after chromatography (petroleum ether/ethyl acetate, 87.5:12.5), 1.17 g (50%) of aldehyde 14d as a pale orange solid: ¹H NMR (300 MHz,CDCl₃) δ 0.83 (t, J=7.4 Hz,3H),1.02.1.09 (m,2H),1.14 (s, 3H),1.18.1.28 (m,2H),1.25 (s,6H),1.43 (s,3H),1.52.1.60 (m,2H), 1.76.1.94 (m,2H),2.05.2.19 (m,1H), 2.53.2.58 (m,1H),2.61 (td, J=4.5, 11.2 Hz,1H),3.74 (dd, J=3.6, 16.1 Hz,1H),3.83 (s,3H),6.39 (d, J=1.6 Hz,1H),6.43 (d, J=1.6 Hz,1H),6.83.6.84 (m, 1H),9.50 (s,1H); ¹³C NMR (75 MHz,CDCl₃) 514.1, 18.2,23.4,26.9,27.6,28.7,28.8,29.2,30.8,37.9,44.2, 45.0,47.3,55.1,75.8, 100.7, 108.1, 110.2, 142.5, 148.9, 150.2,153.7,158.6,193.8; MS (EI)m/z 370 (35),314 (100), 300 (25).

1-Methoxy-3-(1′,1′-dimethylpentyl)-11-hydroxy-Δ⁸-THC (11d). Luche reduction of 0.500 g (1.35 mmol)of aldehyde 14d by the procedure described above for the reduction of 14c gave after chromatography (petroleum ether/ethyl acetate,4:1) 0.350 g (70%)of 11-hydroxy cannabinoid 11d as a pale yellow gum: ¹H NMR (300 MHz,CDCl₃) δ 0.83 (t, J=7.3 Hz,3H),1.00.1.10 (m,2H), 1.11 (s,3H),1.16.1.28 (m,2H),1.24 (s,6H), 1.39 (s,3H),1.49.1.57 (m,2H),1.77.1.91 (m,3H), 2.20.2.25 (m,1H),2.66 (td, J=4.6, 10.9 Hz,1H),3.31 (dd, J=3.6, 17.1 Hz,1H),3.80 (s,3H),4.05 (s,2H),5.73 (d, J=4.2 Hz,1H),6.37 (d, J=1.6 Hz,1H),6.42(d, J=1.6 Hz,1H); ¹³C NMR (75.5 MHz,CDCl₃) δ 14.1, 18.4,23.4, 26.9, 27.6, 27.7, 28.8, 31.5, 31.8,37.6, 44.2,45.2,67.1, 76.3, 100.6, 108.2, 111.1, 120.7, 138.5, 149.8, 153.8,158.6;[αD²⁰ −284° (c=0.25, CHCl₃); HRMS calcd for C₂₄H₃₆O₃: 372.2664, found 372.2663.

1-Methoxy-3-(1′,1′-dimethylhexyl)-11-oxo-Δ⁸-THC (14e). Selenium dioxide oxidation of 2.25 g (6.07 mmol) of 6e, using the procedure described above for the preparation of 10b gave 1.40 g (60%)of aldehyde 14e as an orange solid, mp 117-119° C.; ¹H NMR (300 MHz, CDCl₃) δ 0.83 (t, J=7.1 Hz,3H),1.02.1.30 (m,6H), 1.10 (s,3H),1.25 (s,6H),1.43 (s,3H),1.51.1.56 (m, 2H),1.80.1.93 (m,2H),2.05.2.18 (m,1H),2.49.2.56 (m,1H),2.62 (td, J=4.5, 11.2 Hz,1H),3.73 (dd, J=4.1, 15.7 Hz,1H),3.82(s,3H),6.39 (d, J=1.6 Hz,1H),6.42 (d, J=1.6 Hz,1H),6.84 (br.s,1H), 9.50 (s,1H);¹³C NMR (75.5 MHz,CDCl₃) δ 14.1,18.2, 22.5, 24.3, 27.5, 28.7, 28.8, 29.3, 30.9, 32.5, 37.7, 44.5, 44.9, 55.2,75.8, 100.7, 108.1, 110.3, 142.5, 148.9, 150.2, 153.7, 158.7, 193.8; MS (EI) m/z 384 (20), 314 (100).

1-Methoxy-3-(1′,1′-dimethylhexyl)-11-hydroxy-Δ⁸-THC (11e). Luche reduction of 0.404 g (1.05 mmol)of aldehyde 14e by the procedure described above for the reduction of 14c gave after chromatography (petroleum ether/ethyl acetate, 4:1) 0.354 g (87%)of 11-hydroxy cannabinoid 11e as a pale yellow oil: ¹H NMR (300 MHz,CDCl₃) δ 0.83 (t, J=7.3 Hz,3H),1.07 .1.34 (m,6H),1.11 (s,3H), 1.25 (s,6H), 1.40 (s,3H), 1.51.1.58 (m,2H),1.78.1.88 (m,3H),2.21 .2.25 (m,1H),2.67 (td, J=4.6, 10.9 Hz,1H),3.31 (dd, J=3.5, 17.2 Hz,1H), 3.81 (s,3H), 4.06 (s,2H),5.73 (d, J=4.4 Hz,1H),6.38 (d, J=1.6 Hz,1H),6.43 (d, J=1.6 Hz,1H); ¹³C NMR (75.5 MHz,CDCl₃) δ 14.1, 18.4, 22.6, 24.3, 27.6, 27.7, 28.8, 28.9, 31.5, 31.9, 32.6, 44.4, 45.2, 55.2, 67.2,76.3, 100.7, 108.3, 111.2, 120.8, 138.6, 149.9, 154.0, 158.7; HRMS calcd for C₂₅H₃₈O₃ 386.2821, found 386.2818.

3-(1′,1′-Dimethylheptyl)-1-methoxy-11-oxo-Δ⁸-THC (14f). Selenium dioxide oxidation of 1.64 g (4.27 mmol) of 4, using the procedure described above for the preparation of 10b gave 1.12g (66%) of aldehyde 14f as an orange solid: mp 118-119° C.; R_{f} 0.31 (petroleum ether/ethyl acetate, 94:6); ¹H NMR (300 MHz,CDCl₃) δ 0.84 (t, J=6.9 Hz,3H),1.06.1.27 (m,8H),1.14 (s,3H),1.24 (s,6H),1.43 (s,3H),1.51.1.57 (m,2H),1.66.1.68 (m, 1H),1.88 (td, J=4.1, 11.6 Hz,1H),2.07.2.18 (m,1H), 2.51.2.57 (m,1H),2.61 (td, J=4.1, 11.2 Hz,1H),3.73 (dd, J=4.5, 17.1 Hz,1H),3.81 (s,3H),6.39 (d, J=1.6 Hz,1H),6.42(d, J=1.6 Hz,1H),6.83 (s,1H), 9.50 (s,1H);¹³C NMR (75.5 MHz,CDCl₃) δ 14.1, 18.2, 22.6, 24.6, 27.5, 28.7, 28.9, 29.2, 30.0, 30.8, 31.7, 37.7, 44.5, 45.0, 55.1, 75.8, 100.7, 108.1, 110.2, 142.5, 148.9, 150.2, 153.7, 158.6, 193.8; MS (EI)m/z 398 (25), 314 (50), 281 (66), 207 (100).

11-Hydroxy-3-(1′,1′-dimethylheptyl)-1-methoxy-D 8-THC (11f).Luche reduction of 0.314 g (0.789 mmol)of aldehyde 14f by the procedure described above for the preparation of 14c gave after chromatography (petroleum ether/ethyl acetate, 85:15) 0.270 g (93%)of 11-hydroxy cannabinoid 11f as a pale yellow oil: R_{f} 0.30 (petroleum ether/ethyl acetate,85:15); ¹H NMR (300 MHz,CDCl₃) δ 0.84 (t, J=6.9 Hz, 3H), 0.95.1.26 (m, 8H), 1.10 (s, 3H), 1.24 (s, 6H), 1.39 (s, 3H), 1.51.1.56 (m, 2H), 1.78.1.90 (m, 4H), 2.19.2.26 (m, 1H), 2.66 (td, J=4.1, 10.8 Hz,1H), 3.30 (dd, J=3.3, 16.6 Hz,1H), 3.79 (s,3H), 4.03 (br s,2H), 5.71 (s,1H), 6.37 (d, J=1.6 Hz,1H), 6.42(d, J=1.6 Hz,1H); ¹³C NMR (75.5 MHz,CDCl₃) δ 14.0,18.3, 22.6,24.5, 27.5,27.6, 28.7, 28.8, 29.6, 29.9, 31.4, 31.7, 37.6, 44.4, 45.1, 55.0, 66.9, 76.3, 100.6, 108.2, 111.1, 120.5, 138.5, 149.7, 153.8, 158.6; [αD²⁰ −175° (c=0.3,CH₂Cl₂); HRMS calcd for C₂₆H₄O₃: 400.2977, found 400.2979.

Receptor Binding Assays

1.CB₁ assay.[³H] CP-55,940 (K_{D}32 690 nM) binding to P2 membranes was conducted as described elsewhere,³¹ except whole brain (rather than cortex only) was used. Displacement curves were generated by incubating drugs with 1 nM of [³H]CP-55,940. The assays were performed in triplicate, and the results represent the combined data from three individual experiments.

2.CB₂assay. Human embryonic kidney 293 cells were maintained in Dulbecco's modified Eagle's medium (DMEM) with 10% fetal clone II (HyClone, Logan Utah)and 5% CO₂ at 37° C. in a Forma incubator. Cell lines were created by transfection of CB₂ pcDNA3into 293 cells by the Lipofectamine reagent (Life Technologies, Gaithersburg, Md.).The human CB₂ cDNA was a gift. Stable transformants were selected in growth medium containing geneticin (1 mg/mL, reagent, Life Technologies, Gaithersburg, Md.). Colonies of about 500 cells were picked (about 2 weeks post transfection) and allowed to expand, then tested for expression of receptor mRNA by northern blot analysis. Cell lines containing moderate to high levels of receptor mRNA were tested for receptor binding properties. Transfected cell lines were maintained in DMEM with 10% fetal clone II plus 0.3-.0.5 mg/mL geneticin and 5% CO₂ at 37° C. in a Forma incubator.

The current assay is a modification of Compton et al.⁸ Cells were harvested in phosphate-buffered saline containing 1 mM EDTA and centrifuged at 500 g. The cell pellet was homogenized in 10 mL of solution A (50 mM Tris-HCl, 320 mM sucrose, 2 mM EDTA, 5 mM MgCl₂, pH 7.4). The homogenate was centrifuged at 1600 g (10 min),the supernatant saved, and the pellet washed three times in solution A with subsequent centrifugation. The combined supernatants were centrifuged at 100,000 g (60 min). The (P2 membrane) pellet was resuspended in 3 mL of buffer B (50 mM Tris-HCl, 1 mM EDTA, 3 mM MgCl₂, pH 7.4) to yield a protein concentration of approximately 1 mg/mL. The tissue preparation was divided into equal aliquots, frozen on dry ice, and stored at 70° C. Binding was initiated by the addition of 40-50 μg membrane protein to silanized tubes containing [³H] CP-55,940 (102.9 Ci/mmol) and a suffcient volume of buffer C (50 mM Tris-HCl,1 mM EDTA,3 mM MgCl₂, and 5 mg/mL fatty acid free BSA, pH 7.4) to bring the total volume to 0.5 mL. The addition of 1 m unlabelled CP-55,940 was used to assess nonspecific binding. Following incubation (30° C. for 1 h),binding was terminated by the addition of 2 mL of ice cold buffer D (50 mM Tris-HCl, pH 7.4, plus 1 mg/mL BSA) and rapid vacuum filtration through Whatman GF/C filters (pretreated with polyethyleneimine (0.1%) for at least 2 h).Tubes were rinsed with 2 mL of ice cold buffer D, which was also filtered, and the filters subsequently rinsed twice with 4 mL of ice cold buffer D. Before radioactivity was quantitated by liquid scintillation spectrometry, filters were shaken for 1 h in 5 mL of scintillation fluid.

CP-55,940 and all cannabinoid analogues were prepared by suspension in assay buffer from a 1 mg/mL ethanolic stock without evaporation of the ethanol (final concentration of no more than 0.4%). When anandamide was used as a displacing ligand, experiments were performed in the presence of phenylmethylsulfonyl fluoride (50 μM). Competition assays were conducted with 1 nM [³H] CP-55,940 or 1 nM [³H] SR141716A and 6 concentrations (0.1 nM to 10 m displacing ligands). Displacement IC₅₀values were originally determined by unweighted least-squares linear regression of log concentration-percent displacement data and then converted to Kᵢ values using the method of Cheng and Prusoff.³²

### REFERENCES AND NOTES FOR EXAMPLE 1

1. Matsuda, L. A.; Lolait, S. J.; Brownstein, M. J.; Young, A. C.; Bonner, T. H. Nature 1990, 346, 561.
2. Herkenham, M.; Lynn, A. B.; Little, M. D.; Johnson, M. R.; Melvin, L. S.; De Costa, D. R.; Rice, K. C. Proc. Natl. Acad. Sci. U.S.A. 1990, 87, 1932.
3. Pertwee, R. G. Curr. Med. Chem. 1999, 6, 635.
4. Huffman, J. W.; Lainton, J. A. H. Curr. Med. Chem. 1996, 3,101.
5.Munro, S.; Thomas, K. L.; Abu-Shar, M. Nature (London) 1993, 365, 61.
6. Breivogel, C. S.; Griffn, G.; Di Marzo, V.; Martin, B. R. Mol. Pharmacol. 2001, 155.
7. Little, P. J.; Compton, D. R.; Johnson, M. R.; Melvin, L. S.; Martin, B. R. J. Pharmacol. Exp. Ther. 1988, 247, 1046.
8.Compton, D. R.; Rice, K. C.; De Costa, B. R.; Razdan, R. K.; Melvin, L. S.; Johnson, M. R.; Martin, B. R. J. Pharmacol. Exp. Ther. 1993, 265, 218.
9.Compton, D. R.; Aceto, M. D.; Lowe, J.; Martin, B. R. J. Pharmacol. Exp. Ther. 1996, 277, 586.
10. Zimmer, A.; Zimmer, A. M.; Hohmann, A. G.; Herkenham, M.; Bonner, T. L. Proc. Natl. Acad. Sci. U.S.A. 1999, 96, 5780.
11. Klein, T. W.; Friedman, H.; Specter, S. J Neuroimmunol. 1998, 83, 102.
12. Buckley, N. E.; McCoy, K. L.; Mezey, E.; Bonner, T.; Zimmer, A.; Felder, C. C.; Glass, M.; Zimmer, A. Eur. J. Pharmacol. 2000, 396, 141.
13. Griffin, G.; Wray, E. J.; Tao, Q.; McAllister, S. D.; Rorrer, W. K.; Aung, M. M.; Martin, B. R.; Abood, M. E. Eur. J. Pharmacol. 1999, 317, 117.
14. Lu, Q.; Straiker, A.; Lu, Q.; Maguire, G. Vis. Neurosci. 2000, 17, 91.
15. Baker, D.; Pryce, G.; Croxford, J. L.; Brown, P.; Pertwee, R. G.; Huffman, J. W.; Layward, L. Nature (London) 2000, 404, 84.
16. Sanchez, C.; de Ceballos, M. L.; Gomez del Pulgar, T.; Rueda, D.; Corbacho, C.; Velasco, G.; Galve-Roperh, I.; Huffman, J. W.; Ramon y Cajal, S.; Guzman, M. Cancer Res. 2001, 61, 5784.
17. Malan, T. P.; Ibrahim, M. M.; Deng, H.; Liu, Q.; Mata, H. P.; Vanderah, T.; Porreca, F.; Makriyannis, A. Pain 2001, 93, 239.
18. Recht, L. D.; Salmonsen, R.; Rosetti, R.; Jang, T.; Pipia, G.; Kubiatowski, T.; Karim, P.; Ross, A. H.; Zurier, R.; Litofsky, N. S.; Burstein, S. Biochem. Pharmacol. 2001, 62, 755.
19. Huffman, J. W.; Yu, S.; Showalter, V.; Abood, M. E.; Wiley, J. L.; Compton, D. R.; Martin, B. R.; Bramblett, R. D.; Reggio, P. H. J. Med. Chem. 1996, 39, 3875.
20. Huffman, J. W.; Liddle, J.; Yu, S.; Aung, M. M.; Abood, M. E.; Wiley, J. L.; Martin, B. R. Bioorg. Med. Chem. 1999, 7, 2905.
21. Gareau, Y.; Dufresne, C.; Gallant, M.; Rochette, C.; Sawyer, N.; Slipetz, D. M.; Tremblay, N.; Weech, P. K.; Metters, K. M.; Labelle, M. Bioorg. Med. Chem. Lett. 1996, 6, 189.
22. Hanus, L. R.; Fride, E. Proc. Natl. Acad. Sci. U.S.A. 1999, 96, 14228.
23. Ross, R. A.; Brockie, H. C.; Stevenson, L. A.; Murphy, V. L.; Templeton, F.; Makriyannis, A.; Pertwee, R. G. Br. J. Pharmacol. 1999, 126, 665.
24. Dominianni, S. J.; Ryan, C. W.; De Armitt, C. W. J. Org. Chem. 1977, 42, 344.
25. Petrzilka, T.; Sikemeier, C. Helv. Chim. Acta 1967, 50, 1416.
26. (a) Ben Zvi, Z.; Mechoulam, R.; Burstein, S. H. Tetrahedron Lett. 1970, 4495. (b) Inayama, S.; Sawa, A.; Hosoya, E. Chem. Pharm. Bull. 1974, 22, 1519.©) Mahadevan, A.; Siegel, C.; Martin, B. R.; Abood, M. E.; Belatskaya, I.; Razdan, R. K. J. Med. Chem. 2000, 43, 3778.
27. Gemal, A. L.; Luche, J.-L. J. Am. Chem. Soc. 1981, 103, 5454.
28. Mechoulam, R.; Lander, N.; Breuer, A.; Zahalka, J. Tetrahedron Asymmetry 1990, 1, 315.
29. (a) Liddle, J.; Huffman, J. W.; Wiley, J. L.; Martin, B. R. Bioorg. Med. Chem. Lett. 1998, 8, 2223.(b) Liddle, J.; Huffman, J. W. Tetrahedron 2001, 57, 7607.
30. Showalter, V. M.; Compton, D. R.; Martin, B. R.; Abood, M. E. J. Pharmacol. Exp. Ther. 1996,278,989.
31. Martin, B. R.; Compton, D. R.; Thomas, B. F.; Prescott, W. R.; Little, P. J.; Razdan, R. K.; Johnson, M. R.; Melvin, L. S.; Mechoulam, R.; Ward, S. J. Pharmacol. Biochem. Behav. 1991, 40,471.
32. Cheng, Y. C.; Prusoff, W. H. Biochem. Pharmacol. 1973, 22, 3099.

### EXAMPLE 2

### Structure-Activity Relationships for 1-Alkyl-3-(1-naphthoyl)indoles at the CB₁ and CB₂ receptors: Steric and Electronic Effects of Naphthoyl Substituents. New Highly Selective CB₂ receptor Ligands

Abstract: The synthesis, CB₁ and CB₂ receptor affinities of 47 cannabimimetic indoles are described. The indole derivatives include 1-propyl- and 1-pentyl-3-(1-naphthoyl)indoles both with and without a 2-methyl substituent. Naphthoyl substituents include 4- and 7-alkyl groups as well as 2-, 4-, 6-, 7-methoxy and 4-ethoxy groups. The effects of these substituents on receptor affinities are discussed and structure-activity relationships are presented. In the course of this work three new highly selective ligands for the CB₂ receptor were identified, 1-propyl-3-(4-methyl-1-naphthoylindole (JWH-120), 1-propyl-2-methyl-3-(6-methoxy-1-naphthoylindole (JWH-151), and 1-pentyl-3-(2-methoxy-1-naphthoylindole (JWH-267).

Introduction

Nearly 40 years ago Gaoni and Mechoulam reported the elucidation of the structure of Δ⁹-tetrahydrocannabinol (1, THC, Scheme 1) the principal psychoactive compound present in marijuana.¹ Subsequently, a comprehensive set of structure-activity relationships (SAR) were developed based on the dibenzopyran nucleus of THC.²⁻⁶ These SAR were later extended to the very potent group of non-traditional cannabinoids developed by Pfizer, of which CP-55,940 (2, DMH=1,1-dimethylheptyl, Scheme 1) is the prototypical example.^{7,8}

These pharmacological effects of cannabinoids are considered to be mediated through at least two G-protein-coupled, transmembrane receptors. One of these, designated as CB₁, is found predominantly in the central nervous system and is thought to be responsible for most of the overt pharmacological effects of cannabinoids.^{5,9-11} A second receptor, designated CB₂ was originally identified from macrophages present in the spleen, and is expressed primarily in the periphery.¹² Very recently evidence has been presented for the existence of a third cannabinoid receptor, which has been detected in mouse brain.¹³

Some years ago in the course of a program directed toward the development of nonsteroidal anti-inflammatory drugs, a group at Sterling-Winthrop reported that pravadoline (3), an indole derivative, unexpectedly inhibited contractions of the electrically stimulated mouse vas deferens.¹⁴ Later work revealed that 3 (Scheme 1), and a number of related compounds also inhibit adenylate cyclase, are antinociceptive and interact with a G-coupled protein in the brain. Subsequent work indicated that the G-coupled protein is the cannabinoid CB₁ receptor and that these aminoalkylindoles exhibit typical cannabinoid pharmacology in vivo.^{15,16} One of the aminoalkylindoles, WIN-55,212-2 (4, Scheme 1), developed by the Winthrop group is not only potent in vivo, but has high affinity for both the cannabinoid CB₁ and CB₂ receptors.¹⁷

During the course of their investigations of the aminoalkylindoles, the Winthrop group synthesized more than 100 compounds and developed some preliminary SAR.^{14,15,18} These included the observation that a group larger than methyl at C-2 of the indole nucleus greatly attenuates potency and that a bicyclic aroyl group, usually 1-naphthoyl or a substituted 1-naphthoyl group, at C-3 is essential for potency. It was further concluded that an aminoalkyl group, usually substituted aminoethyl, attached to the indole nitrogen was essential for cannabinoid activity.

Subsequent studies by our group established that the aminoalkyl group appended to the indole nitrogen could be replaced by an alkyl group to provide relatively simple indole derivatives which exhibit typical cannabinoid pharmacology.^{19,20} In particular, JWH-007, 1-pentyl-2-methyl-3-(1-naphthoyl)indole (5, Scheme 1) has high affinity for the CB₁ receptor and exhibits typical cannabinoid pharmacology in vivo. The 1-propyl analog of 5, JWH-015 (6, Scheme 1) has relatively high affinity for the CB₂ receptor, and weak affinity for the CB₁ receptor.¹⁷

In an effort to develop improved indole based CB₂ cannabinoid receptor ligands and also to develop SAR for both the CB₁ and CB₂ receptors a number of additional indole derivatives were prepared and their pharmacology was evaluated.^{21,22} It was found that CB₁ receptor affinity is optimal with a n-pentyl nitrogen substituent, and decreases dramatically with N-alkyl substituents of three or less carbon atoms. CB₁ receptor affinity is also greatly attenuated with N-alkyl substituents longer than six carbon atoms. A 2-methyl substituent slightly decreases affinity at the CB₁ receptor and a 4-methoxy-1-naphthoyl group at C-3 of the indole, as in JWH-098 (7) enhances CB₁ receptor affinity. A 7-methyl-1-naphthoyl substituent in JWH-046 (8) and JWH-048 (9) has relatively little effect on either CB₁ or CB₂ receptor affinity. The SAR at the CB₂ receptor are somewhat similar to those at the CB₁ receptor, however there are a number of exceptions to these generalizations which render it difficult to establish comprehensive SAR for these compounds at the CB₂ receptor.^{21,23}

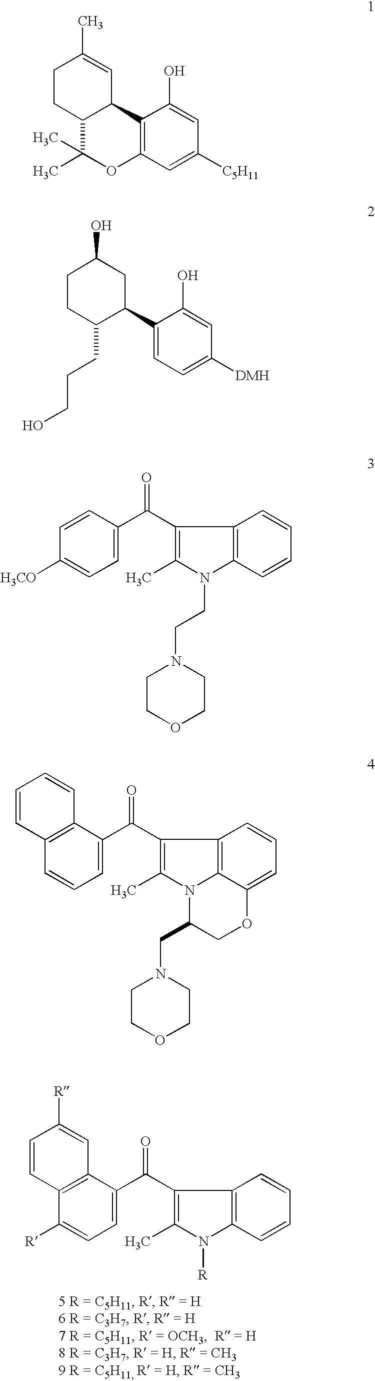

### Formulas 1-9

The SAR outlined above includes only indoles with an unsubstituted 3-naphthoyl substituent, or with 4-methoxy- or 7-methyl-1-naphthoyl groups. To further develop SAR for these compounds, and with the goal of preparing CB₂ selective ligands, a number of additional cannabimimetic indoles have been prepared and their affinities for the CB₁ and CB₂ receptors have been determined. These compounds include N-propyl- and N-pentyl-3-(1-naphthoyl) indoles in which the naphthoyl group contains various alkyl and alkoxy substitutuents, and the indole is either unsubstitued at C-2 or contains a 2-methyl group. The choice of the N-propyl substituent is based on the observation that the two most highly CB₂ selective indole derivatives prepared in our laboratory, JWH-015 (6) and JWH-046 (8) both contain this substitution pattern.^{21,23} The N-pentyl group was chosen since this substituent almost invariably provides compounds with higher CB₁ receptor affinities than are observed with other nitrogen substituents.

Results

To explore the steric and electronic effects of various naphthoyl substituents, 3-(4-alkyl-1-naphthoyl), 3-(7-ethyl-1-naphthoyl), 3-(2-, 6-,7-methoxy-1-naphthoyl), 3-(4-ethoxy-1-naphthoyl)indoles (for numbering see the structure included in Table 2), have been synthesized and their CB₁ and CB₂ receptor affinities have been determined. These indoles were prepared by modifications of established routes, either from substituted 1-naphthoyl chlorides and 1-alkyl- or 1-alkyl-2-methylindoles or by acylation of indole or 2-methylindole, followed by N-alkylation (Scheme 1).^{14,15,18,19,24}

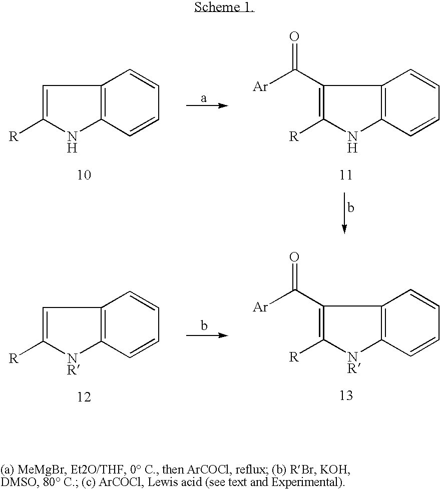

TABLE 2
Structures, Method of Synthesis and Receptor Affinities (mean ± SEM) of 3-(Alkyl-1-naph-
thoyl)indoles.

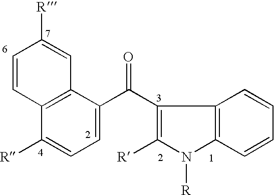

| | | | | | | Kᵢ (nm) | | |
|---|---|---|---|---|---|---|---|---|
| | | | | | | | | Ratio |
| Compound | Synthesis | R | R′ | R″ | R′′′ | CB₁ | CB₂ | CB₁₇CB₂ |
| Δ⁹-THC (1) | | | | | | 41 ± 2^{a} | 36 ± 10^{b} | 1.1 |
| WIN-55,212-2 | | | | | | 1.9 ± 0.1^{b} | 0.28 ± 0.16^{b} | 6.8 |
| (4) | | | | | | | | |
| JWH-072^{c} | | C₃H₇ | H | H | H | 1050 ± 55^{c} | 170 ± 54^{c} | 6.2 |
| JWH-015^{c,d} (6) | | C₃H₇ | CH₃ | H | H | 164 ± 22^{c} | 13.8 ± 4.6^{b} | 11.9 |
| JWH-018^{c} | | C₅H₁₁ | H | H | H | 9 ± 5^{c} | 2.9 ± 2.6^{c} | 3.3 |
| JWH-007^{c,d} (5) | | C₅H₁₁ | CH₃ | H | H | 9.5 ± 4.5^{c} | 2.0 ± 2.6^{c} | 3.3 |
| JWH-120 (41) | B | C₃H₇ | H | CH₃ | H | 1054 ± 31 | 6.1 ± 0.7 | 173 |
| JWH-148 | A | C₃H₇ | CH₃ | CH₃ | H | 123 ± 8 | 14 ± 1.0 | 8.8 |
| JWH-122^{e} | | C₅H₁₁ | H | CH₃ | H | 0.69 ± 0.5^{e} | 1.2 ± 1.2 | 0.6 |
| JWH-149^{e} | C | C₅H₁₁ | CH₃ | CH₃ | H | 5.0 ± 2.1^{e} | 0.73 ± 0.03 | 6.8 |
| JWH-212 | C | C₃H₇ | H | C₂H₅ | H | 33 ± 0.9 | 10 ± 1.2 | 3.3 |
| JWH-211 | C | C₃H₇ | CH₃ | C₂H₅ | H | 70 ± 0.8 | 12 ± 0.8 | 5.8 |
| JWH-210 | C | C₅H₁₁ | H | C₂H₅ | H | 0.46 ± 0.03 | 0.69 ± 0.01 | 0.67 |
| JWH-213 | C | C₅H₁₁ | CH₃ | C₂H₅ | H | 1.5 ± 0.2 | 0.42 ± 0.05 | 3.6 |
| JWH-180 | C | C₃H₇ | H | C₃H₇ | H | 26 ± 2 | 9.6 ± 2.0 | 2.7 |
| JWH-189 | C | C₃H₇ | CH₃ | C₃H₇ | H | 52 ± 2 | 12 ± 0.8 | 4.3 |
| JWH-182 | C | C₅H₁₁ | H | C₃H₇ | H | 0.65 ± 0.03 | 1.1 ± 0.1 | 0.6 |
| JWH-181 | C | C₅H₁₁ | CH₃ | C₃H₇ | H | 1.3 ± 0.1 | 0.62 ± 0.04 | 2.1 |
| JWH-239 | C | C₃H₇ | H | C₄H₉ | H | 342 ± 20 | 52 ± 6 | 6.6 |
| JWH-241 | C | C₃H₇ | CH₃ | C₄H₉ | H | 147 ± 20 | 49 ± 7 | 3.0 |
| JWH-240 | C | C₅H₁₁ | H | C₄H₉ | H | 14 ± 1 | 7.2 ± 1.3 | 1.9 |
| JWH-242 | C | C₅H₁₁ | CH₃ | C₄H₉ | H | 42 ± 9 | 6.5 ± 0.3 | 6.5 |
| JWH-076 | A | C₃H₇ | H | H | CH₃ | 214 ± 11 | 106 ± 46 | 2.0 |
| JWH-046f (8) | | C₃H₇ | CH₃ | H | CH₃ | 343 ± 38^{f} | 16 ± 5^{f} | 21 |
| JWH-248f (9) | | C₅H₁₁ | CH₃ | H | CH₃ | 10.7 ± 1.0^{f} | 0.49 ± 0.1^{f} | 22 |
| JWH-235 | B | C₃H₇ | H | H | C₂H₅ | 338 ± 34 | 123 ± 34 | 2.7 |
| JWH-236 | B | C₃H₇ | CH₃ | H | C₂H₅ | 1351 ± 204 | 240 ± 63 | 5.6 |
| JWH-234 | B | C₅H₁₁ | H | H | C₂H₅ | 8.4 ± 1.8 | 3.8 ± 0.6 | 2.2 |
| JWH-262 | B | C₅H₁₁ | CH₃ | H | C₂H₅ | 28 ± 3 | 5.6 ± 0.7 | 5.0 |

^{a}Ref 41.
^{b}Ref 17.
^{c}Refs. 20 and 21.
^{d}Ref. 19.
^{e}Ref. 24.
^{f}Ref. 21.

In our early work, indole (10, R═H) or 2-methylindole (10, R═CH₃) was treated with methylmagnesium bromide to give the ambident indolyl anion which upon reaction with an aroyl chloride gave the 3-acylindole (11, method A).¹⁹ N-Alkylation with the appropriate primary alkyl bromide using KOH in DMSO provides the N-alkyl-3-aroylindole. Although this sequence was satisfactory when using readily available aroyl halides, the yields in the first step were variable, the 3-aroylindoles (11) were difficult to purify and alternative methodology was explored. Traditional aluminum chloride catalyzed Friedel-Crafts acylation of N-alkylindoles gave poor yields, however two variations of this classical reaction gave satisfactory to good yields. The method of choice is that reported recently by Okauchi et al. (method B) in which N-alkylindoles 12 are treated with dimethyl- or diethylaluminum chloride for 30 minutes prior to the addition of the aroyl halide.²⁵ This mild procedure provides pure products (13) in yields of 50% to 90%. The final methods are variations of a classical Friedel-Crafts procedure, but use ethylaluminum dichloride at ambient temperature for either four days (method C) or for 18 h (method D). The yields for procedures C and D are adequate, but are somewhat inferior to those using Okauchi's method (procedure B).

Of the 1-naphthoic acids required for the synthesis of the cannabimimetic 3-(4-alkyl-1-naphthoyl)indoles, only 4-methyl-1-naphthoic acid (14, Scheme 2) is commercially available. 4-Ethyl-(15), 4-propyl-(16) and 4-butyl-1-naphthoic (17) acids were reported some years ago by Russian workers, who prepared them by chloromethylation of the 1-alkylnaphthalene, followed by conversion to the corresponding acetate, hydrolysis to the alcohol and oxidation to the carboxylic acid.²⁶ A considerably more efficient approach employs Friedel-Crafts acylation of the appropriate 1-alkylnaphthalene (18, R═C₂H₅, C₃H₇, C₄H₉) with N,N,-diphenylcarbamyl chloride to give 1-naphthyl N,N,-diphenylamides (19, R═C₂H, C₃H₇, C₄H₉), hydrolysis of which will afford the corresponding carboxylic acid.²⁷ Although the acylation step proceeded smoothly in yields of 80% to 85%, hydrolysis could not be effected using the reported procedure, ethanolic sodium hydroxide at reflux. The amides were successfully hydrolyzed in 90% to 92% yield by treatment with potassium hydroxide in diethylene glycol at reflux.^{28,29} naphthoic acids (14-17). Conversion of 4-methyl-1-naphthoic acid to 1-propyl-3-(4-methyl-1-naphthoyl)indole (JWH-120) and 2-methyl-1-propyl-3-(4-methyl-1-naphthoyl)indole (JWH-148) was carried out using methods B and A, respectively. The 1-pentyl analogs of these compounds (JWH-122, JWH-149) have been reported previously.²⁴ The conversion of acids 14-17 to the 3-(4-ethyl-1-naphthoyl)indoles (JWH-210-JWH-213), the 3-(4-propyl-1-naphthoyl)indoles (JWH-180-JWH-182, JWH-189) and the 3-(4-butyl-1-naphthoyl)indoles (JWH-239-JWH-242) was carried out using method C. The CB₁ and CB₂ receptor affinities of these compounds are summarized in Table 2. 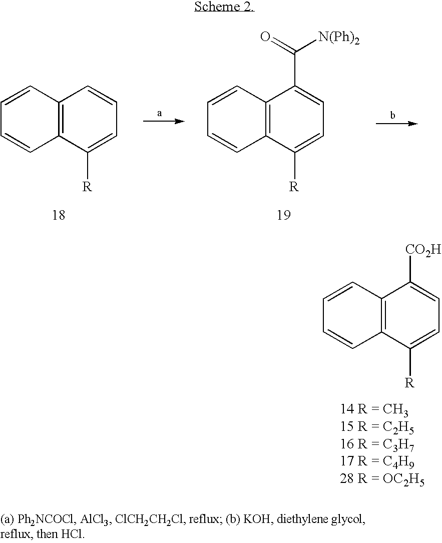
7-Methyl-1-naphthoic acid (20), which had been used previously for the synthesis of JWH-046 (8) and JWH-048 (9),²¹ was prepared by a modification of the method of Snatzke and Kunde (Scheme 3).³⁰ Friedel-Crafts acylation of 2-methylnaphthalene (21) under standard conditions using acetyl chloride and AlCl₃ proceeds as described to provide 1-acetyl-7-methylnaphthalene (22), however, conversion to acid 20 could not be effected using the haloform reaction under various conditions. In the King modification of the haloform reaction, ketone 22 with iodine and pyridine gave pyridinium iodide 23, which upon basic hydrolysis and acidification provided acid 20.³¹ Conversion of this acid to 1-propyl-3-(7-methyl-1-naphthoyl)indole was carried out by method A. 7-Ethyl-1-naphthoic acid (24), which was employed for the synthesis of the 3-(7-ethyl-1-naphthoyl)indoles was prepared from 2-ethylnaphthalene (25) in an analogous manner through ketone 26 and pyridinium iodide 27. Conversion of 7-ethyl-1-naphthoic acids to the 3-(7-ethyl-1-naphthoyl)indoles (JWH-234-JWH-236, JWH-262) was carried out using method B. The receptor affinities for the 3-(7-alkyl-1-naphthoyl)indoles are included in Table 2.

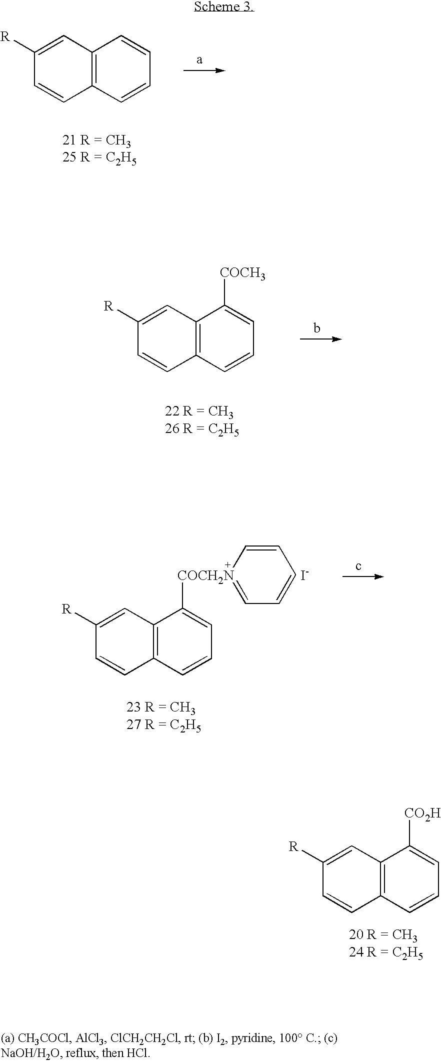

In the methoxynaphthoyl series, the 2-, 4-, 6- and 7-methoxy-1-naphthoylindoles were prepared and their affinities for the CB₁ and CB₂ receptors were determined (Table 3). In addition, to evaluate steric and electronic effects upon receptor affinities, the 4-ethoxy analogs were also prepared. The 3-(4-ethoxy-1-naphthoyl)indoles were prepared from 4-ethoxy-1-naphthoic acid (28, Scheme 2), which was obtained from 1-ethoxynaphthalene by the procedure outlined in Scheme 2. Acid 28 was converted to the 3-(4-ethoxy-1-naphthoyl)indoles (JWH-258-JWH-261) by method B and their receptor affinities are included in Table 3. The 3-(4-methoxy-1-naphthoyl)indoles, JWH-079, JWH-094, JWH-081, JWH-098, have been reported previously and their affinities for the CB₁ and CB₂ receptors are also included in Table 2.^{21,24}

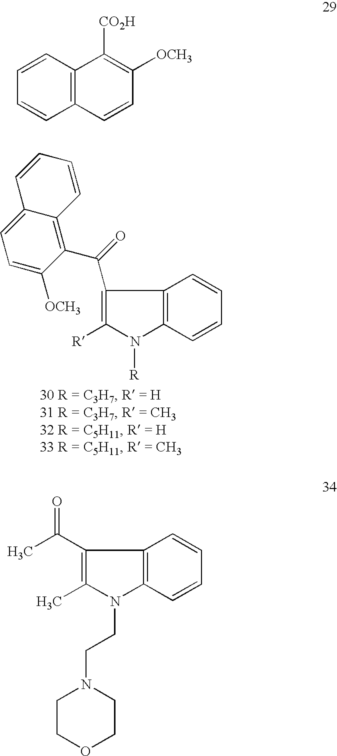

TABLE 3
Structures, Method of Synthesis and Receptor Affinities (mean ± SEM) of 3-(Alkoxy-1-naphthoyl)indoles.

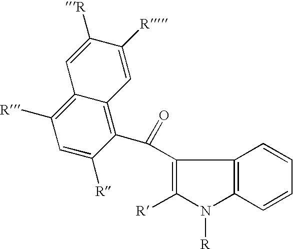

| Compound | Synthesis | R | R′ | R″ | R′′′ | R′′′′ | R′′′′′ | CB₁ | CB₂ | Ratio CB₂/CB₁ |
|---|---|---|---|---|---|---|---|---|---|---|
| Δ9-THC (1) | | | | | | H | H | 41 ± 2^{a} | 36 ± 10^{b} | 1.1 |
| WIN-55,212-2 (4) | | | | | | H | H | 1.9 ± 0.1^{b} | 0.28 ± 0.16^{b} | 6.8 |
| JWH-265 (30) | B | C₃H₇ | H | OCH₃ | H | H | H | 3788 ± 323 | 80 ± 13 | 47 |
| JWH-266 (31) | B | C₃H₇ | CH₃ | OCH₃ | H | H | H | >10,000 | 455 ± 55 | >22 |
| JWH-267 (32) | B | C₅H₁₁ | H | OCH₃ | H | H | H | 381 ± 16 | 7.2 ± 0.14 | 53 |
| JWH-268 (33) | B | C₅H₁₁ | CH₃ | OCH₃ | H | H | H | 1379 ± 193 | 40 ± 0.6 | 34 |
| JWH-079c | | C₃H₇ | H | H | OCH₃ | H | H | 63 ± 3^{c} | 32 ± 6^{c} | 2.0 |
| JWH-094^{c} | | C₃H₇ | CH₃ | H | OCH₃ | H | H | 476 ± 67^{c} | 97 ± 3^{c} | 4.9 |
| JWH-081^{c,d} | | C₅H₁₁ | H | H | OCH₃ | H | H | 1.2 ± 0.03^{c,d} | 12.4 ± 2.2^{c} | 0.1 |
| JWH-098^{c,d} | | C₅H₁₁ | CH₃ | H | OCH₃ | H | H | 4.5 ± 0.1^{c,d} | 1.9 ± 0.3^{c} | 2.4 |
| JWH-259 | B | C₃H₇ | H | H | OC₂H₅ | H | H | 220 ± 29 | 74 ± 7 | 3.0 |
| JWH-261 | B | C₃H₇ | CH₃ | H | OC₂H₅ | H | H | 767 ± 105 | 221 ± 14 | 3.5 |
| JWH-258 | B | C₅H₁₁ | H | H | OC₂H₅ | H | H | 4.6 ± 0.6 | 10.5 ± 1.3 | 0.44 |
| JWH-260 | B | C₅H₁₁ | CH₃ | H | OC₂H₅ | H | H | 29 ± 0.4 | 25 ± 1.9 | 1.2 |
| JWH-163 | D | C₃H₇ | H | H | H | OCH₃ | H | 2358 ± 215 | 138 ± 12 | 17 |
| JWH-151 (42) | D | C₃H₇ | CH₃ | H | H | OCH₃ | H | >10,000 | 30 ± 1.1 | >333 |
| JWH-166 | D | C₅H₁₁ | H | H | H | OCH₃ | H | 44 ± 10 | 1.9 ± 0.08 | 23 |
| JWH-153 | D | C₅H₁₁ | CH₃ | H | H | OCH₃ | H | 250 ± 24 | 11 ± 0.5 | 23 |
| JWH-165 | D | C₃H₇ | H | H | H | H | OCH₃ | 204 ± 26 | 71 ± 8 | 2.9 |
| JWH-160 | D | C₃H₇ | CH₃ | H | H | H | OCH₃ | 1568 ± 201 | 441 ± 110 | 3.6 |
| JWH-164 | D | C₅H₁₁ | H | H | H | H | OCH₃ | 6.6 ± 0.7 | 6.9 ± 0.2 | 0.96 |
| JWH-159 | D | C₅H₁₁ | CH₃ | H | H | H | OCH₃ | 45 ± 1 | 10.4 ± 1.4 | 4.3 |

^{a}Ref. 41.
^{b}Ref. 17.
^{c}Ref. 21.
^{d}Ref. 24

For the synthesis of the 3-(2-methoxy-1-naphthoyl)indoles 2-methoxy-1-naphthoic acid (29) was required. Acid 29 is readily available 2-hydroxy-1-naphthoic acid 32 and was converted to Indoles 30-33 (JWH-265-JWH-268, Table 3) by method B. Indoles 30 (JWH-265) and (JWH-267) which are unsubstituted at C-2 of the indole nucleus show normal 1H and ¹³C NMR spectra, however in the ¹H spectra of the 2 -methyl analogs (31, JWH-266, and 33, JWH-268) the signal at d 2.50 due to the indole 2-methyl group and an aromatic proton at d 6.95 appeared as very broad singlets. A second aromatic proton at d 7.13 gave rise to a somewhat broadened singlet, rather than the sharp signals characteristic of the naphthoylindoles. Also, the signals due to the 2-methyl group and one aromatic carbon were missing in the ¹³C spectra of indoles 31 and 33. Although it was possible that these compounds did not have the correct structures, both had low and high resolution mass spectra consistent with the assigned molecular formulas. Ultimately the structure of 1-propyl-2-methyl-3-(2-methoxy-1-naphthoyl)indole (31) was established by x-ray crystallography.³³

It appeared probable that the anomalous NMR spectra were due to restricted rotation about the bond between C-3 of the indole nucleus and/or the naphthalene C-1 bond and the carbonyl carbon atom. Accordingly, a variable temperature study of the ¹H NMR spectrum of indole 31 was carried out. There was little change in the ¹H spectrum at 50° C., but at 75° C. the signal for the C-2 methyl group had sharpened somewhat, as had that of the aromatic proton at d 6.95, and the signal at d 7.13 was resolved to a broadened triplet, J=8.2 Hz. At 125° C. the C-2 methyl signal was a rather sharp singlet at d 2.46, the aromatic proton at d 6.95 had become a well defined triplet, J=7.8 Hz, and the triplet at d 7.13 appeared as a triplet of doublets, J=0.5 and 8.2 Hz. The ¹H spectrum at 100° C. was slightly less well resolved than that at 125° C.

A COSY spectrum of the aromatic region of indole 31 revealed that the protons which give rise to the triplets at d 6.95 and d 7.13 are coupled to each other and each is coupled to one additional proton within the complex pattern between d 7.3 and v 7.6. The triplets at d 6.95 and d 7.13 can only arise from the 5,6-protons on the indole nucleus or the 6,7-naphthalene protons. Since the aromatic protons of a simple acyl indole derivative, 2-methyl-1-morpholinoethyl-3-acetylindole (34) appear as a series of multiplets between d 7.26 and d 7.97,³⁴ the triplets at d 6.95 and d 7.13 in indole 31 must arise from the protons at C-6 and C-7 of the naphthalene. These data confirm that the anomalies in the NMR spectra of indoles 31 and 33 are the result of restricted rotation about the bond between C-3 of the indole nucleus and/or the naphthalene C-1 bond and the carbonyl carbon atom. The energy barrier to rotation was determined using variable temperature ¹H NMR, and the coalescence temperature was found to be 0° C. The free energy of activation was calculated from the stopped exchange limit at 31 50° C. and the coalescence temperature to give a rotational barrier of 13.1 kcal/mol.³⁵ An AMI coordinate drive study shows that during rotation about the naphthalene C-1 bond and the carbonyl carbon atom the oxygen of the methoxy group has close Van der Waals' contacts with C-2 and C-3 of the indole. The maximum overlap occurs at a carbonyl oxygen-carbonyl carbon-indole C-3-indole C-2 torsion angle of −89.8°. The calculated energy difference between the global minimum energy conformer and this conformation is 9.38 kcal/mol. Single point energy calculations for the difference in energy between the global minimum energy conformer and the conformation for which maximum overlap occurs was found to be 12.86 kcal/mol at the HF 3-21G* level. The receptor affinities of the 3-(2-methoxy-1-naphthoyl)indoles (JWH-265-JWH-268) are included in Table 3.

The 6- and 7-methoxy-1-naphthoylindoles required 6-(35, Scheme 4) and 7-methoxy-1-naphthoic acid (36), respectively, both of which were prepared a number of years ago using classical chemistry. The 6-methoxy acid (35) was originally synthesized by Price et al. in 12% yield by the aluminum chloride catalyzed reaction of furoic acid and anisole.³⁶ 7-Methoxy-1-naphthoic acid (36) was described by Fieser and Holmes who prepared it from ethyl 4-(4-methoxyphenyl)butanoate via 7-methoxy-3,4-dihydro-1-naphthoic acid.³⁷ The dihydro acid was synthesized in 51% overall yield, however no yield was specified for the dehydrogenation to acid 36. A subsequent synthesis of acid 36 utilized the reaction of 2-methoxynaphthalene with an oxalyl chloride equivalent to give a methoxyacenapthenquinone, which was converted to acid 36 in two steps and good yield.³⁸ In our hands attempted repetition of the reaction of 2-methoxynaphthalene with the oxalyl chloride surrogate failed. Other published syntheses of both acids did not appear attractive and alternative methods of synthesis were explored.

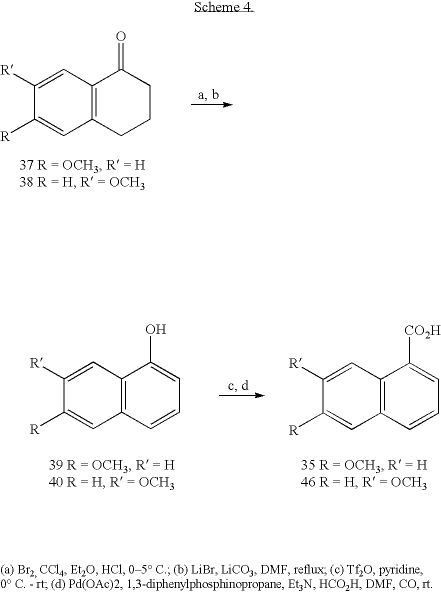

Both 6-(37) and 7-methoxy-1-tetralone (38, Scheme 4) are commercially available, and appeared to be suitable starting materials for the synthesis of acids 35 and 36. The initial synthetic route to 6-methoxy-1-naphthoic acid (35) entailed conversion of tetralone 37 to the enol triflate, followed by palladium mediated carbonylation to 6-methoxy-3,4-dihydro-1-naphthoic acid.³⁹ Dehydrogenation to acid 35 did not proceed to completion and the mixture of 35 and the dihydro acid proved exceedingly difficult to separate. The conversion of tetralones 37 and 38 to 6-(39) and 7-methoxy-1-naphthol (40), respectively was reported some years ago by Shand and Thompson who accomplished this transformation by dehydrogenation using sulfur at 250° C.⁴⁰ As an alternative to thermal dehydrogenation, ketones 37 and 38 were converted to the corresponding a-bromo ketones, dehydrohalogenation of which provided phenols 39 and 40.⁴¹ Conversion of these phenols to the corresponding triflates followed by palladium mediated carbonylation gave acids 35 and 36 in good yield. Acid 35 was converted to the 3-(6 -methoxy-1-naphthoyl)indoles (JWH-151, JWH-153, JWH163, JWH-166) and acid 36 was converted to the 3-(7-methoxy-1-naphthoyl)indoles (JWH-159, JWH-160, JWH-164, JWH-165) by method D. The receptor affinities for these 3-(6- and 7-methoxy-1-naphthoyl)indoles are included in Table 3.

The affinities of the alkylnaphthoyl and alkoxynaphthoylindoles for the CB₁ receptor were determined by measuring their ability to displace the potent cannabinoid [³H] CP-55,940 (2) from its binding site in a membrane preparation from rat brain as described by Compton et al.⁴² Affinities for the CB₂ receptor were determined by measuring the ability of the compounds to displace [³H] CP-55,940 from a cloned human receptor preparation using the procedure described by Showalter et al.¹⁷ The results of these determinations are summarized in Tables 2 and 3. Also included in Tables 2 and 3 are the receptor affinities for Δ⁹-THC (1) and WIN55,212-2 (4).

In the 4-alkyl-1-naphthoyl series (Table 2), the 4-methyl-1-propyl compounds, JWH-120 (41) and JWH-148, have very slight (Kᵢ=1054±31 nM) and modest (Kᵢ=123±8 nM) affinities respectively for the CB₁ receptor. This pair of compounds is unusual in that the analog with an indole 2-methyl group (JWH-148) has significantly greater affinity for the CB₁ receptor than the unsubstituted compound (JWH-120). The CB₂ receptor affinities of these compounds are similar, for JWH-120 Kᵢ=6.1±7 nM, and for JWH-148 Kᵢ=14±1.0 nM. The 4-methyl-1-pentyl analogs, JWH-122 and JWH-149 have high affinities for both the CB₁ and CB₂ receptors. In particular, JWH-122, the compound lacking the indole C-2 methyl group, has subnanomolar affinity for the CB₁ receptor (Kᵢ=0.69±0.5 nM).²⁴

The 4-ethyl- and 4-propyl-1-naphthoylindoles (Table 2) all have uniformly high affinities for both receptors. Following the usual trend for CB₁ receptor affinities the 1-propyl compounds have lower affinities than the 1-pentyl analogs, and the compounds with an indole 2-methyl group have somewhat lower affinities than the unsubstituted analogs. The CB₁ receptor affinities for these compounds range from Kᵢ=0.46±0.03 nM for 1-pentyl-3-(4-ethyl-1-naphthoyl)indole, JWH-210, to Kᵢ=70±0.8 nM for 2-methyl-1-propyl-3-(4-ethyl-1-naphthoyl)indole, JWH-211. The CB₁ receptor affinities for the 3-(4-propyl-1-naphthoyl) indoles are comparable to those of the 4-ethyl analogs. The CB₂ receptor affinities for these eight compounds fall within a narrow range, from Kᵢ=0.62±0.04 for 2-methyl-1-pentyl-3-(4-propyl-1-naphthoyl)indole, JWH-181, to Kᵢ=12±0.8 for 2-methyl-1-propyl-3-(4-ethyl-1-naphthoyl)indole, JWH-211.

The 4-butyl-1-naphthoylindoles (Table 1) have significantly lower affinities for the CB₁ receptor than the 4-ethyl and 4-propyl-1-naphthoylindoles. In particular, the affinities of 1-propyl-3-(4-butyl-1-naphthoyl)indole, JWH-239 (Kᵢ=342±20 nM) and its 2-methylindole analog, JWH-241 (Kᵢ=147±20 nM) are significantly lower than the ethyl and propyl analogs. The CB₁ receptor affinities of the 1-pentyl-3-(4-butyl-1-naphthoyl)indoles are much higher than those of the 1-propyl compounds. 1-Pentyl-3-(4-butyl-1-naphthoyl)indole, JWH-240 has Kᵢ=14±1 nM and the 2-methylindole analog, JWH-242, has Kᵢ=42±9 nM. Again, the values for the CB₂ receptor affinities of the 4-butyl-1-naphthoylindoles are not nearly as diverse as the CB₁ affinities. The 1-propyl analogs, JWH-239 and JWH-241 have Kᵢ=52±6 nM and Kᵢ=49±7 nM, respectively. The CB₂ receptor affinities for the 1-pentyl compounds, JWH-240 and JWH-242 are essentially identical with Kᵢ=7.2±13 nM for JWH-240 and Kᵢ=6.5±0.3 nM for JWH-242.

We had previously reported the CB₁ and CB₂ receptor affinities of 2-methyl-1-propyl-3-(7-methyl-1-naphthoyl)indole (8), JWH-046 with Kᵢ=343±38 nM at CB₁ and Kᵢ=16±5 nM at CB₂ (Table 2), which is comparable in its affinity for both receptors and in CB₂ selectivity to JWH-015 (6).²¹ JWH-076, the analog of JWH-046 lacking the 2-methyl group, has slightly greater affinity for the CB₁ receptor than JWH-046 (Kᵢ=214±11 nM), but considerably less affinity for the CB₂ receptor (Kᵢ=106±46 nM). 2-Methyl-1-pentyl-3-(7-methyl-1-naphthoyl) indole (9, JWH-048) was reported previously and has Kᵢ=10.7±1 nM for the CB₁ receptor and Kᵢ=0.49±0.1 nM for the CB₂ receptor.²¹ These values are very similar to those of the analog without the 7-methyl group, JWH-007 (5), which has Kᵢ=9.5±4.5 nM at the CB₁ receptor and Kᵢ=2.9±2.6 nM at the CB₂ receptor.¹⁹⁻²¹

For the 1-propyl-3-(7-ethyl-1-naphthoyl)indoles (Table 2), the 2-methyl compound, JWH-236 has considerably lower affinity than its 7-methyl homologue for both the CB₁ and CB₂ receptors with Kᵢ=1351±204 nM at CB₁ and Kᵢ=240±63 nM at CB₂. The analog lacking the 2-methyl group, JWH-235 has receptor affinities very similar to those of the 7 -methyl compound, with Kᵢ=338±34 nM at CB₁ and Kᵢ=123±34 nM at CB₂. The 1-pentyl compounds both have considerably greater affinities for both receptors than the 1-propyl analogs. For 1-pentyl-3-(7-ethyl-1-naphthoyl)indole, JWH-234, Kᵢ=8.4±1.8 nM at CB₁ and Kᵢ=3.8±0.6 nM at CB₂. The 2-methyl analog has slightly less affinity for both receptors with Kᵢ=28±3 nM at CB₁ and Kᵢ=5.6±0.7 nM at CB₂.

In the 3-(4-methoxy-1-naphthoyl)indole series the receptor affinities of all four of the 1-propyl- and 1-pentyl-3-(4-methoxy-1-naphthoyl)indoles have been reported previously (Table 2).^{21,24} The 1-propyl-2-methyl analog (JWH-094) has little affinity for the CB₁ receptor with Kᵢ=476±67 nM, and only modest affinity for the CB₂ receptor (Kᵢ=97±3 nM). However, JWH-079, 1-propyl-3-(4-methoxy-1-naphthoyl)indole has Kᵢ=63±3 nM at CB₁ and good affinity for the CB₂ receptor (Kᵢ=32±6 nM). The 1-pentyl analogs have considerably higher affinity for both receptors. 1-Pentyl-3-(4-methoxy-1-naphthoyl)indole (JWH-081) has very high affinity for the CB₁ receptor (Kᵢ=1.2±0.03 nM) and high affinity for the CB₂ receptor (Kᵢ=12.4±2.2 nM). The 2-methyl analog (JWH-098, 7) has slightly lower affinity for the CB₁ receptor with Kᵢ=4.5±0.1 nM and somewhat higher affinity for the CB₂ receptor (Kᵢ=1.9±0.3 nM).

The 3-(4-ethoxy-1-naphthoyl)indoles have from somewhat to considerably lower affinity for both receptors than the 4-methoxy analogs (Table 3). At the CB₁ receptor the 1-propyl compounds have Kᵢ=220±29 nM and Kᵢ=767±105 nM, respectively, for 1-propyl-3-(4-ethoxy-1-naphthoyl)indole (JWH-259) and 2-methyl-1-propyl-3-(4-ethoxy-1-naphthoyl)indole (JWH-261). JWH-259 has modest affinity (Kᵢ=74±7 nM), while the 2-methyl analog (JWH-261) has even less affinity (Kᵢ=221±14 nM) at the CB₂ receptor. The 1-pentyl compounds have considerably greater affinity for both receptors than the corresponding 1-propylindoles. 1-Pentyl-3-(4-ethoxy-1-naphthoyl)indole (JWH-258) has Kᵢ=4.6±0.6 nM at CB₁ and Kᵢ=10.5 ±1.3 nM at the CB₂ receptor. The 2-methyl compound, 2-methyl-1-pentyl-3-(4-ethoxy-1-naphthoyl)indole (JWH-260) has Kᵢ=29±0.4 nM at CB₁ and Kᵢ=25±1.9 nM at the CB₂ receptor.

None of the 1-alkyl-3-(2-methoxy-1-naphthoyl)indoles (JWH-265-JWH-268, Table 2) have significant affinity for the CB₁ receptor, with Kᵢ=381±16 nM for 1-pentyl-3-(2 -methoxy-1-naphthoyl)indole (JWH-267) to Kᵢ=>10,000 nM for the 1-propyl-2-methyl analog (JWH-266). The CB₂ receptor affinities of this series of compounds are considerably greater than for the CB₁ receptor. 1-Propyl-3-(2-methoxy-1-naphthoyl)indole (JWH-265) has moderate affinity, Kᵢ=80±13 nM, for the CB₂ receptor while the 2-methyl analog (JWH-266) has little affinity for the receptor, Kᵢ=455±55 nM. 1-Pentyl-3-(2-methoxy-1-naphthoyl)indole (JWH-267) has very high affinity, Kᵢ=7.35 0.14 nM, for the CB₂ receptor and the 2-methyl compound (JWH-268) has somewhat less affinity with Kᵢ=40±1 nM.

The only 6-methoxy-1-naphthoylindole with significant affinity for the CB₁ receptor is 1-pentyl-3-(6-methoxy-1-naphthoyl)indole (JWH-166, Table 2) which has good affinity with Kᵢ=44±10 nM. The affinities of the other three members of this series for the CB₁ receptor range from Kᵢ=240±24 nM for 2-methyl-1-pentyl-3-(6-methoxy-1-naphthoyl)indole (JWH-153) to Kᵢ=>10,000 for the 1-propyl-2-methyl analog (JWH-151, 42). The 1-pentyl-3-(6-methoxy-1-naphthoyl)indoles (JWH-153, JWH-166) have similar and high CB₂ affinities with Kᵢ=11±1 nM and Kᵢ=1.9±0.1 nM, respectively. 1-Propyl-3-(6-methoxy-1-naphthoyl)indole (JWH-163) has Kᵢ=138±12 nM at CB₂, while the 2-methyl compound (JWH-151, 42) has significant affinity for the CB₂ receptor, Kᵢ=30±1 nM. JWH-151 is a highly selective (>333-fold) ligand for the CB₂ receptor with effectively no affinity for the CB₁ receptor.

In the 7-methoxy-1-naphthoyl series (Table 3), neither of the 1-propyl compounds has high affinity for either the CB₁ or CB₂ receptor (Table 3). 1-Propyl-3-(7-methoxy-1-naphthoyl)indole (JWH-165) has Kᵢ=204±26 nM at the CB₁ receptor and Kᵢ=71±8 nM at the CB₂ receptor. The 2-methyl analog (JWH-160) has Kᵢ=1568±201 nM at CB₁ and Kᵢ=441 ±110 mM at CB₂. In contrast, 1-pentyl-3-(7-methoxy-1-naphthoyl)indole (JWH-164) has Kᵢ=6.6±0.7 nM at the CB₁ receptor and Kᵢ=6.9±0.2 nM at the CB₂ receptor. The compound with the 2-methyl group, 2-methyl-1-pentyl-3-(7-methoxy-1-naphthoyl)indole (JWH-159), has somewhat less affinity for the CB₁ receptor than JWH-164 with Kᵢ=45±1 nM and similar affinity for the CB₂ receptor, Kᵢ=10.4±1.4 nM.

Three of the indole derivatives synthesized in the course of this SAR study are highly selective for the CB₂ receptor. These are 1-propyl-3-(4-methyl-1-naphthoyl)indole, JWH-120 (41) which is 173-fold selective, 1-pentyl-3-(2-methoxy-1-naphthoyl)indole, JWH-267 (32), 53 -fold selective and 1-propyl-2-methyl-3-(6-methoxy-1-naphthoyl)indole, JWH-151 (42) which is >333 fold selective. In order to evaluate the efficacy of these compounds, their ability to stimulate GTPγS binding was determined. This is a functional assay which measures G-protein coupled receptor activation using [³⁵S]GTPγS binding.⁴³ Chinese Hamster Ovary (CHO) cells stably expressing the human CB₂ receptor were employed in this determination (see Experimental). The results of these determinations are summarized in Table 4. The stimulation is normalized to that produced by 3 mM CP-55,940 (2), a maximally effective concentration of this standard cannabinoid agonist. In addition to indoles 32, 41 and 42 the [³⁵S]GTPγS binding for JWH-015, 1-propyl-2-methyl-3-(1-naphthoyl)indole (6), the lead compound for the search for CB₂ selective cannabimimetic indoles, was determined, and the data are included in Table 3. Also included in Table 4 are data for two dibenzopyran-based cannabinoids which are highly selective for the CB₂ receptor, 1-deoxy-3-(1′,1′-dimethylbutyl)-Δ⁸-THC (JWH-133, 43) which has excellent affinity for the CB₂ receptor (Kᵢ=3.4±1.0 nM) and little affinity for the CB₁ receptor (Kᵢ=677±132 nM)⁴⁴ and 1-methoxy-3-(1′,1′-dimethylhexyl)-Δ⁸-THC (JWH-229, 44) with Kᵢ=18±2 nM at CB₂ and Kᵢ=3134±110 nM at CB₁.⁴⁵

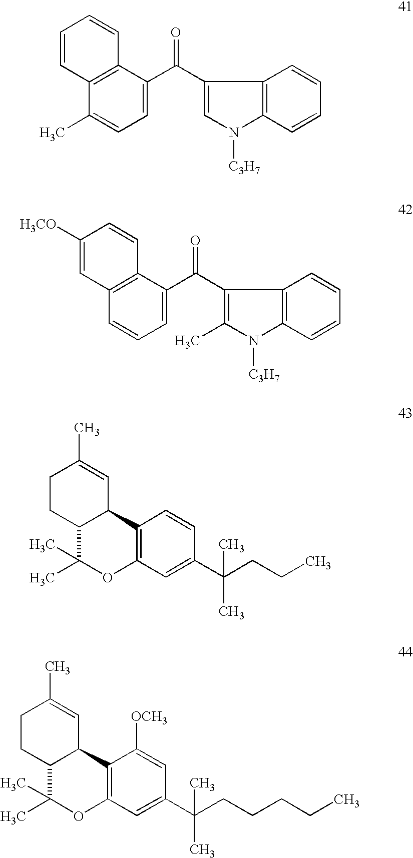

TABLE 4
EC₅₀ and Eₘₐₓ Values (mean ± SEM) for GTPγS
Binding of CB₂ for Selective Ligands. Assays were carried out
in Human CB₂ Receptor-Expressing CHO Cells.
Eₘₐₓ Values are Reported as Per Cent
Relative to 3 μM CP-55, 940 (2).

| | | Eₘₐₓ |
|---|---|---|
| Compound | EC₅₀ (nM) | (% CP-55940) |
| 2-Methyl-1-propyl-3-(1-naphthoyl)indole(JWH-015, 6) | 17.7 ± 1.0 | 65.7 ± 6.4 |
| 1-Deoxy-3-(1′,1′-dimthylbutyl)-Δ⁸-THC (JWH-133, 43) | 4.0 ± 1.0 | 111.5 ± 13.6 |
| 1-Methoxy-3-(1′,1′-dimthylhexyl)-Δ⁸-THC (JWH-229, 44) | 4.6 ± 2.0 | 75.7 ± 8.3 |
| 1-Propyl-3-(4-methyl-1-naphthoyl)indole(JWH-120, 41) | 5.1 ± 1.6 | 78.1 ± 10.7 |
| 1-Pentyl-3-(2-methoxy-1-naphthoyl)indole(JWH-267, 32) | 4.9 ± 0.8 | 67.3 ± 2.9 |
| 1-Propyl-2-methyl-3-(6-methoxy-1-naphthoyl)indole | 12.0 ± 2.9 | 108.5 ± 13.0 |
| (JWH-151, 42) | | |

As indicated in Table 4 all six of these compounds are potent in the [³⁵S]GTPγS assay with EC₅₀ values from 4.0±1.0 nM for JWH-133 (43) to 17.7±1.0 nM for JWH-015 (6). Two of these CB₂ receptor ligands, JWH-133 (43) and 1-propyl-2-methyl-3-(6-methoxy-1-naphthoyl)indole, JWH-151 (42) are highly efficacious with Eₘₐₓ values of 111.5±13.6% and 108.5±13.0% respectively, relative to CP-55,940., The other three cannabimimetic indoles, 1-propyl-2-methyl-3-(1-naphthoyl)indole, JWH-015 (6), 1-propyl-3-(4-methyl-1-naphthoyl)indole, JWH-120 (41) and 1-pentyl-3-(2-methoxy-1-naphthoyl)indole, JWH-267 (32), plus 1-methoxy-3-(1′,1′-dimethylhexyl)-Δ⁸-THC, JWH-229 (44) are all partial agonists relative to CP-55,940 with Eₘₐₓ values from 65.7±6.4% (JWH-015) to 78.1±10.7% (JWH-120).

Conclusions

The CB₁ receptor affinities for 1-pentyl- and 1-propyl-3-(1-naphthoyl)indoles with 4- and 7-alkylnaphthoyl and 2-, 4-methoxy, 6-, 7-methoxy and 4-ethoxynaphthoyl substituents indicate that receptor affinity is enhanced considerably by the presence of small alkyl groups (methyl, ethyl, propyl) at C-4. Methyl or ethyl substituents at C-7 of the naphthoyl group have little effect on affinity relative to the unsubstitued compounds. A methoxy substituent at C-4 enhances CB₁ receptor affinity, while a 4-ethoxy substituent has relatively little effect on affinity. 6-Methoxy-1-naphthoylindoles have attenuated CB₁ receptor affinities relative to the unsubstitued compounds, while a 7-methoxy substituent has little effect upon affinity. A 2 -methoxy substituent effectively destroys affinity for the CB₁ receptor. These conclusions have been rationalized in terms of molecular modeling and receptor docking studies.

The CB₂ receptor affinities of these naphthoylindoles show considerably less variation than their CB₁ affinities. Only four compounds with alkylnaphthoyl substituents have CB₂ receptor affinities greater than 52 nM. Three of these are 1-propyl-3-(7-methyl or 7-ethyl-1-naphthoyl)indoles and the fourth is 1-propyl-3-(-1-naphthoyl)indole. In the alkoxynaphthoyl series there is no clear pattern. Most of the alkoxynaphthoyl cannabimimetic indoles have good to moderate affinity for the CB₂ receptor, however a 1-propyl-2-methylindole substitution pattern leads to attenuated affinity in several cases.

A 2-methyl substituent on the indole nucleus results in a decrease in affinity for either receptor relative to the unsubstituted analog. Similarly, the 1-propylindoles in general have lower affinities at both receptors than the 1-pentyl compounds. However, the differences in CB₂ receptor affinities as a function of N-substitution are less pronounced than is the case with CB₁ affinities. Although three new cannabimimetic indoles with potentially useful CB₂ selectivity have been identified, there do not appear to be sufficient data to permit the rational design of additional indole based CB₂ selective ligands.

Experimental

General. IR spectra were obtained using Nicolet 5DX or Magna spectrometers; 1H and ¹³C NMR spectra were recorded on a Bruker 300AC spectrometer. Mass spectral analyses were performed on a Hewlett-Packard 5890A capillary gas chromatograph equipped with a mass sensitive detector. HRMS data were obtained in the Mass Spectrometry Laboratory, School of Chemical Sciences, University of Illinois. Ether and THF were distilled from Nabenzophenone ketyl immediately before use, and other solvents were purified using standard procedures. Column chromatography was carried out on Sorbent Technologies silica gel (32-63 μ) using the indicated solvents as eluents. All new compounds were homogeneous to TLC and ¹³C NMR. All target compounds were homogeneous to GLC or TLC in two different solvent systems. TLC was carried out using 200 mm silica gel plates using the indicated solvents. GLC analyses were performed on the Hewlett-Packard 5890A GC/MS using a 60 m carbowax column and helium gas as a carrier. An initial column temperature of 60° C. was employed and the temperature was increased at a rate of 1.5° C./min to a maximum temperature of 300° C. with a total run time of 20 min. Elemental analyses were performed by Atlantic Microlab, Norcross, Ga.

1-Propyl-3-(4-methyl-1-naphthoyl)indole JWH-120 (41, Method B). To a stirred solution of 0.080 g (0.50 mmol) of 1-propylindole in 1.5 mL of dry CH₂Cl₂ at 0° C. under N₂ was added dropwise 0.75 mL (0.75 mmol) of Me₂AlCl (1 M in hexanes). The solution was stirred at 0° C. for 30 min and a solution of 0.122 g (0.6 mmol) of freshly prepared 4-methyl-1-naphthoyl chloride in 1.5 mL of CH₂Cl₂. The acid chloride was prepared form 0.122 g (0.60 mmol) of 4-methyl-1-naphthoic acid to which was added dropwise 1 mL of thionyl chloride at a rate sufficient to maintain a steady evolution of gas. This solution was heated at reflux for 15 min, cooled to ambient temperature and the excess thionyl chloride was removed in vacuo to give the acid chloride which was used without further purification. The deep red acylation reaction mixture was stirred at 0° C. until the reaction was complete as indicated by tlc (approximately 1 h). The reaction mixture was poured carefully into iced 1 M aqueous HCl and extracted with three portions of CH₂Cl₂. The combined extracts were washed wth three portions of aqueous NaHCO₃, dried (MgSO₄) and the solvent was removed in vacuo to give the crude product. After chromatography (petroleum ether:ethyl acetate, 9: 1) there was obtained 0.140 g (86%) of indole 41 as a white solid. Recrystallization from hexanes/ethyl acetate gave the analytical sample: m.p. 207-208° C.; ¹H NMR (300 MHz, CDCl₃) δ 0.90 (t, J=7.3 Hz, 3H), 1.81-1.88 (m, 2H), 2.78 (s, 3H), 4.03 (t, J=7.0 Hz, 2H), 7.37-7.38 (m, 5H), 7.48-7.59 (m, 3H), 8.09 (d, J=8.2 Hz, 1H), 8.27 (d, J=8.2 Hz, 1H), 8.52-8.54 (m, 1H); ¹³C NMR (75.5 MHz, CDCl₃) δ 11.3, 19.7, 23.1, 48.7, 109.9, 117.6, 122.7, 122.9, 123.5, 124.2, 125.5, 125.8, 126.1, 126.3, 126.6, 127.0, 130.9, 132.8, 136.6, 137.0, 137.5, 137.9, 192.2; Anal. Calcd for C₂₃H₂₁NO: C, 84.37; H, 6.46; N, 4.28; Found: C, 84.25; H, 6.46; N, 4.30.

2-Methyl-1-propyl-3-(4-methyl-1-naphthoyl)indole JWH-148 (Method A). To a stirred solution of 14.1 mL (42.3 mmol) of 3.0 M MeMgBr in ether, diluted with 20 mL of THF, at 0° C. was added 4.62 g (35.2 mmol) of 2-methylindole in 10 mL of THF. The reaction mixture was allowed to warm to ambient temperature and a solution of 4-methyl-1-naphthoyl chloride (from 6.0 g, 35 mmol, of 4-methyl-1-naphthoic acid) in 5 mL of THF was added dropwise. The mixture was heated at reflux for 1.5 h and the reaction was quenched by the cautious addition of saturated aqueous NH₄Cl. Stirring was continued until the solid precipitate was broken up and the solid was collected, and suspended in 200 mL of methanol. A solution of 3 g of KOH in 20 mL of water was added and the mixture was heated at reflux for 4 h. The precipitate was filtered off and dried to give 3.7 g (35%) of 2-methyl-3-(4-methyl-1-naphthoyl)indole was added to a stirred suspension of 1.48 g of powdered KOH in 5 mL of DMSO and 1.44 g (11.7 mmol) of 1-bromopropane was added. The reaction mixture was stirred at 80° C. for 18 h, poured into water and extracted with three portions of ethyl acetate. The combined extracts were washed with water, dried (Na₂SO₄) and the solvent was removed to give the crude product. Chromatography (petroleum ether:ethyl acetate, 10:1) gave 1.62 g (81%) of JWH-148 as a white solid: m.p. 135-137°; ¹H NMR (300 MHz, CDCl₃) δ 0.95 (t, J=7.4 Hz, 3H), 1.79 (sextet, J=7.4 Hz, 2H), 2.44 (s, 3H), 2.74 (s, 3H), 4.04 (t, J=7.3 Hz, 2H), 6.98 (t, J=7.9 Hz, 1H), 7.15 (t, J=7.9 Hz, 1H), 7.21-7.52 (m, 6H), 8.06 (d, J=8.5 Hz, 1H), 8.18 (d, J=8.3 Hz, ¹H); ¹³C NMR (75.5 MHz, CDCl₃) δ 11.6, 12.7, 20.0, 23.0, 44.9, 109.6, 115.2, 121.4, 121.9, 122.2, 124.4, 125.9, 126.2, 126.4, 126.6, 127.3, 130.6, 133.0, 136.2, 136.8, 139.0, 145.5, 193.7; MS (EI) m/z 341 (80), 326 (43), 324 (25), 141 (100); Anal. Calcd for C₂₄H₂₃NO: C, 84.42; H, 6.79; N, 4.10; Found: C, 84.39;H, 6.83; N, 4.08.

4-Ethyl-1-N,N-dipehylamidonaphthalene (19, R═C₂H₅). A mixture of 3.00 g, (19.2 mmol) of 1-ethylnaphthalene, 4.45 g (19.2 mmol) of diphenylcarbamyl chloride and 2.82 g (21.1 mmol) of anhydrous AlCl₃ in 38.4 mL of 1,2-dichloroethane was heated at reflux for 6 h. After cooling, the reaction was poured onto a mixture of ice and concentrated HCl and extracted with ether. The extracts were dried (MgSO₄) and the solvent was removed in vacuo. The residue was chromatographed (petroleum ether:ethyl acetate, 9:1) to give a white solid. Recrystallization gave 1.72 g (80%) of amide which used in the next step without further purification: m.p. 126-127°; ¹H NMR (300 MHz, CDCl₃) δ 1.29 (t, J=7.5 Hz, 3H), 3.02 (q, J=7.5 Hz, 2H), 7.08-7.31 (m, 12H), 7.49-7.57 (m, 2H), 8.02 (d, J=7.6 Hz, 1H), 8.32 (d, J=7.6 Hz, 1H); ¹³C NMR (75.5 MHz, CDCl₃) δ 14.7, 26.0, 123.4, 124.0, 125.9, 126.1, 126.4, 127.2, 128.9, 131.0, 131.8, 132.6, 142.2, 143.2, 170.6; MS (EI) m/z 351 (12), 183 (100).

4-Ethyl-1-naphthoic acid (15). A mixture of 3.70 g (10.5 mmol) of the above diphenylamide, 23.2 g of KOH and 154 mL of diethylene glycol was heated at reflux for 8 h. The reaction mixture was cooled, diluted with 1.13 L of water and the precipitated solids filtered off. The filtrate was acidified with conc. HCl until precipitation was complete. The solid was collected and dried to give 1.90 g (90%) of acid 15: m.p. 125-127° C. (lit mp 129-130° C., ref. 26): ¹H NMR (300 MHz, CDCl₃) δ 1.44 (t, J=7.3 Hz, 3H), 3.20 (q, J=7.5 Hz, 2H), 7.44 (d, J=7.6 Hz, 1H), 7.59-7.70 (m, 2H), 8.16 (d, J=8.2 Hz, 1H), 8.39 (d, J=7.4 Hz, 1H), 9.21 (d, J=8.6 Hz, 1H); ¹³C NMR (75.5 MHz, CDCl₃) 614.7, 26.5, 123.8, 124.1, 125.0, 126.1, 126.6, 127.5, 131.9, 132.1, 147.5, 173.7

1-Propyl-3-(4-ethyl-1-naphthoyl)indole, JWH-212 (Method C). 4-Ethyl-1-naphthoic acid was converted to the acid chloride by the procedure described above in the preparation of 2-methyl-1-propyl-3-(4-methyl-1-naphthoyl)indole (JWH-148). To a solution of 4-ethyl-1-naphthoyl chloride, from 0.20 g (1.00 mmol) of 4-ethyl-1-naphthoic acid in 4.0 mL of toluene was added a solution of 0.19 g (1.20 mmol) of 1-propylindole. The solution was cooled to 0° C. and 0.26 mL (1.5 mmol) of 1.0 M EtAlCl₂ in hexanes was added dropwise with stirring. The reaction mixture was warmed to ambient temperature and stirred for four days. After the addition of 2.0 mL of water, the layers were separated and the aqueous layer was extracted with ethyl acetate. The combined organic extracts were washed with water, dried (MgSO₄) and concentrated in vacuo. The residue was chromatographed (petroleum ether:ether, 9:1) to give 0.190 g (56%) of JWH-212 as a yellow gum: ¹H NMR (300 MHz, CDCl₃) δ 0.90 (t, J=7.4 Hz, 3H), 1.45 (t, J=7.5 Hz, 3H), 1.78-1.90 (m, 2H), 3.19 (q, J=7.4 Hz, 2H), 4.04 (t, J=7.1 Hz, 2H), 7.35-7.40 (m, 5H), 7.44-7.53 (m, 2H), 7.55-7.61 (m, 2H), 8.14 (d, J=8.4 Hz, 1H), 8.26 (d, J=8.3 Hz, 1H); ¹³C NMR (75.5 MHz, CDCl₃) d 11.3, 14.9, 23.1, 26.1, 48.7, 109.9, 117.7, 122.7, 122.9, 123.5, 123.8, 124.6, 125.9, 126.0, 126.2, 126.8, 127.1, 131.1, 132.0, 137.0, 137.4, 137.9,142.5, 192.6; MS (EI) m/z 341 (100), 324 (65), 312 (91); HRMS Calcd for C₂₄H₂₃NO: 341.1780, Found 341.1779.

1-Pentyl-3-(4-ethyl-1-naphthoyl)indole, JWH-210. JWH-210 was prepared from 4-ethyl-1-naphthoic acid and 1-pentylindole by method C. From 0.20 g (1.00 mmol) of 4-ethyl-1-naphthoic acid and 0.22 g (1.20 mmol) of 1-pentylindole there was obtained after chromatography (petroleum ether:ether, 9:1) 0.193 g (52%) of JWH-210 as a viscous oil: ¹H NMR (300 MHz, CDCl₃) δ 0.88 (t, J=6.9 Hz, 3H), 1.24-1.35 (m, 3H), 1.40-1.49 (m, 4H), 1.76-1.85 (m, 2H), 3.21 (q, J=7.5 Hz, 2H), 4.05 (t, J=7.2 Hz, 2H), 7.35-7.42 (m, 5H), 7.49-7.52 (m 2), 7.55-7.64 (m, 2H), 8.16 (d, J=8.3 Hz, 1H), 8.32 (d, J=8.5 Hz, 1H); ¹³C NMR (75.5 MHz, CDCl₃) 13.8, 14.8,22.1,26.1,28.8,29.4,45.0, 109.9, 117.5, 122.6, 122.8, 123.4, 123.7, 124.0, 125.8, 126.0, 126.1, 126.7, 127.0, 131.1, 131.9, 136.9, 137.4, 137.8, 142.4, 192.2; MS (EI) m/z 369 (100), 352 (64), 312 (88); HRMS Calcd for C₂₆H₂₇NO: 369.2093, Found 369.2090.

2-Methyl-1-propyl-3-(4-ethyl-1-naphthoyl)indole, JWH-211. JWH-211 was prepared from 4-ethyl-1-naphthoic acid and 2-methyl-1-propylindole by method C. From 0.20 g (1.00 mmol) of 4-ethyl-1-naphthoic acid and 0.21 g (1.20 mmol) of 2-methyl-1-propylindole there was obtained after chromatography (petroleum ether:ether, 9:1) 0.192 g (52%) of JWH-211 as a gum: ¹H NMR (300 MHz, CDCl₃) δ 1.00 (t, J=7.4 Hz, 3H), 1.43 (t, J=7.5 Hz, 3H), 1.78-1.90 (m, 2H), 2.48 (s, 3H), 3.20 (q, J=7.5 Hz, 2H), 4.10 (t, J=7.5 Hz, 2H), 7.00 (d, J=7.2 Hz, 1H), 7.15-7.25 (m, 2H), 7.30-7.35 (m, 2H), 7.40-7.55 (m, 3H), 8.14 (d, J=8.4 Hz, 1H), 8.18 (d, J=8.6 Hz, 1H); ¹³C NMR (75.5 MHz, CDCl₃) δ 11.4, 12.5, 14.9, 22.9, 26.2, 44.8, 109.4, 115.1, 121.3, 121.8, 122.1, 123.8, 124.0, 125.8, 126.0, 126.3, 126.4, 127.1, 130.7, 132.1, 136.1, 138.8, 142.7, 145.4, 192.9; MS (EI) m/z 355 (100), 338 (40), 326 (96), 298 (38); HRMS Calcd for C₂₅H₂₅NO: 354.1858, Found 354.1852.

2-Methyl-1-pentyl-3-(4-ethyl-1-naphthoyl)indole, JWH-213. JWH-213 was prepared from 4-ethyl-1-naphthoic acid and 2-methyl-1-propylindole by method C. From 0.20 g (1.00 mmol) of 4-ethyl-1-naphthoic acid and 0.24 g (1.20 mmol) of 2-methyl-1-propylindole there was obtained after chromatography (petroleum ether:ether, 9:1) 0.205 g (55%) of JWH-213 as a gum: ¹H NMR (300 MHz, CDCl₃) δ 0.98 (t, J=7.0 Hz, 3H), 1.22-1.34 (t, J=7.3 Hz, 3H), 1.42-1.49 (m, 4H), 1.84-1.94 (m, 2H), 2.56 (s, 3H), 3.28 (q, J=7.5 Hz, 2H), 4.20 (t, J=7.5 Hz, 2H), 7.10 (d, J=7.3 Hz, 1H), 7.27-7.34 (m, 2H), 7.38-7.54 (m, 2H), 7.59-7.64 (m, 3H), 8.22 (d, J=8.4 Hz, 1H), 8.26 (d, J=8.2 Hz,1H); ¹³C NMR (75.5 MHz, CDCl₃) δ 12.4, 13.8, 14.9, 22.3, 26.1,29.0,29.2,43.2, 109.3, 115.0, 121.2, 121.7, 122.0, 123.8, 123.9, 125.7, 125.9, 126.2, 126.3, 127.1, 130.7, 132.0, 136.0, 138.8, 142.5, 145.2, 193.6; MS (EI) m/z 383 (100), 368 (59), 354 (71), 298 (43); HRMS Calcd for C₂₇H₂₉NO: 383.2249, Found 383.2246.

4-Propyl-1-naphthoic acid (16). Acid 16 was prepared in a manner analogous to that employed for 4-ethyl-1-naphthoic acid: m.p. 134-135° C. (lit mp 141-142° C., ref. 26); ¹H NMR (300 MHz, CDCl₃) 67 1.06 (t, J=7.3 Hz, 3H), 1.78-1.86 (m, 2H), 3.13 (t, J=7.7 Hz, 2H), 7.41 (d, J=7.5 Hz, 1H), 7.56-7.67 (m, 2H), 8.14 (d, J=8.2 Hz, 1H), 8.34 (d, J=7.5 Hz, 1H), 9.16 (d, J=8.6 Hz, 1H); ¹³C NMR (75.5 MHz, CDCl₃) δ 24.2, 23.8,35.7, 123.9, 124.3, 124.9, 126.0, 126.6, 127.5, 131.6, 132.1, 132.3, 146.1, 172.9; MS (ED) m/z 214 (59), 185 (100), 157 (20).

1-Propyl-3-(4-propyl-1-naphthoyl)indole, JWH-180. JWH-180 was prepared from 4-propyl-1-naphthoic acid and 1-propylindole by method C. From 0.11 g (0.51 mmol) of 4-propyl-1-naphthoic acid and 0.098 g (0.62 mmol) of 1-propylindole there was obtained after chromatography (petroleum ether:ether, 9:1) 0.098 g (54%) of JWH-180 as a gum: ¹H NMR (300 MHz, CDCl₃) δ 0.90 (t, J=7.4 Hz, 3H), 1.08 (t, J=7.3 Hz, 3H), 1.80-1.88 (m, 4H), 3.12 (t, J=7.7 Hz, 2H), 4.05 (t, J=7.1 Hz, 2H), 7.34-7.43 (m, 5H), 7.46-7.50 (m,2H), 7.52-7.60 (m, 2H), 8.12 (d, J=8.1 Hz, 1H), 8.24 (d, J=8.1 Hz, 1H); ¹³C NMR (75.5 MHz, CDCl₃) δ 11.2, 14.3, 23.1, 23.8, 35.4, 48.7, 109.9, 117.6, 122.7, 122.9, 123.5, 124.0, 124.6, 125.7, 126.0, 126.1, 126.7, 127.0, 131.2, 132.2, 137.0, 137.5, 137.9, 141.0, 192.0; MS (EI) m/z 355 (100), 338 (57), 326 (52); HRMS Calcd for C₂₅H₂₅NO: 355.1936, Found 355.1924.

1-Pentyl-3-(4-propyl-1-naphthoyl)indole, JWH-182. JWH-182 was prepared from 4-propyl-1-naphthoic acid and 1-pentylindole by method C. From 0.11 g (0.51 mmol) of 4-propyl-1-naphthoic acid and 0.11 g (0.62 mmol) of 1-pentylindole there was obtained after chromatography (petroleum ether:ether, 9:1) 0.099 g (50%) of JWH-182 as a gum: ¹H NMR (300 MHz, CDCl₃) δ 0.86 (t, J=6.8 Hz, 3H), 1.09 (t, J=7.3 Hz, 3H), 1.24-1.32 (m, 4H), 1.79-1.92 (m, 4H), 3.13 (t, J=7.7 Hz, 2H), 4.07 (t, J=7.2 Hz, 2H), 7.35-7.44 (m, 5H), 7.46-7.50 (m, 2H), 7.52-7.60 (m, 2H), 8.13 (d, J=8.4 Hz, 1H), 8.26 (d, J=8.2 Hz, 1H); ¹³C NMR (75.5 MHz, CDCl₃) δ 13.8, 14.3, 22.1, 23.8, 28.9, 29.5, 35.4, 47.1, 109.9, 117.6, 122.7, 122.9, 123.5, 124.0, 124.6, 125.7, 126.0, 126.1, 126.7, 127.0, 131.2, 132.2, 137.0, 137.5, 137.8, 141.0, 192.0; MS (EI) m/z 383 (100), 366 (63), 326 (75); HRMS Calcd for C₂₇H₂₉NO: 383.2249, Found 383.2243.

2-Methyl-1-propyl-3-(4-propyl-1-naphthoyl)indole, JWH-189. JWH-189 was prepared from 4-propyl-1-naphthoic acid and 2-methyl-1-propylindole by method C. From 0.12 g (0.56 mmol) of 4-propyl-1-naphthoic acid and 0.12 g (0.67 mmol) of 2-methyl-1-propylindole there was obtained after chromatography (petroleum ether:ether, 9:1) 0.12 g (60%) of JWH-189 as a gum: ¹H NMR (300 MHz, CDCl₃) δ 0.97-1.08 (m, 6H), 1.80-1.88 (m, 4H), 2.48 (s, 3H), 3.13 (t, J=7.6 Hz, 2H), 4.10 (t, J=7.5 Hz, 2H), 7.00 (d, J=7.6 Hz, 1H), 7.15-7.25 (m, 2H), 7.30-7.38 (m, 2H), 7.40-7.56 (m, 3H), 8.13 (d, J=8.5 Hz, 1H), 8.25 (d, J=8.3 Hz, 1H); ¹³C NMR (75.5 MHz, CDCl₃) δ 11.4, 12.9, 14.2, 22.9, 23.8, 35.4, 44.8, 109.4, 121.3, 122.0, 122.1, 122.9, 124.1, 125.2, 125.7, 126.0, 126.4, 127.4, 128.0, 130.9, 132.3, 136.0, 139.1, 141.1, 145.7, 192.9; MS (EI) m/z 369 (100), 352 (40), 326 (87); HRMS Calcd for C₂₆H₂₇NO: 369.2093, Found 369.2088.

2-Methyl-1-pentyl-3-(4-propyl-1-naphthoyl)indole, JWH-181. JWH-181 was prepared from 4-propyl-1-naphthoic acid and 2-methyl-1-pentylindole by method C. From 0.11 g (0.51 mmol) of 4-propyl-1-naphthoic acid and 0.12 g (0.62 mmol) of 2-methyl-1-pentylindole there was obtained after chromatography (petroleum ether:ether, 9:1) 0.11 g (54%) of JWH181 as a gum: ¹H NMR (300 MHz, CDCl₃) δ 0.93 (t, J=7.4 Hz, 3H), 1.05 (t, J=7.3 Hz, 3H), 1.36-1.40 (m, 4H), 1.80-1.87 (m, 4H), 2.48 (s, 3H), 3.13 (t, J=7.6 Hz, 2H), 4.12 (t, J=7.6 Hz, 2H), 7.00 (d, J=7.5 Hz, 1H), 7.15-7.22 (m, 2H), 7.30-7.35 (m, 2H), 7.40-7.55 (m, 3H), 8.13 (d, J=8.5 Hz, 1H), 8.17 (d, J=8.4 Hz, 1H); ¹³C NMR (75.5 MHz, CDCl₃) δ 12.5, 13.9, 14.2, 22.4, 23.8, 29.1, 29.3, 35.4, 43.3, 109.3, 115.1, 121.3, 121.8, 122.1, 124.1, 125.2, 125.6, 126.3, 126.4, 127.1, 130.8, 132.2, 136.0, 138.8, 141.0, 145.3, 193.8; MS (EI) m/z 397 (100), 382 (56), 354 (81); HRMS Calcd for C₂₈H₃₁NO: 397.2406, Found 397.2394.

4-Butyl-1-naphthoic acid (17). Acid 17 was prepared in a manner analogous to that employed for 4-ethyl-1-naphthoic acid: m.p. 136-137° C. (lit mp 148-148.5° C., ref. 26); ¹H NMR (300 MHz, CDCl₃) δ 0.99 (t, J=7.3 Hz, 3H), 1.42-1.54 (m, 2H), 1.73-1.81 (m, 2H), 3.14 (t, J=7.7 Hz, 2H), 7.41 (d, J=7.3 Hz, 1H), 7.56-7.67 (m, 2H), 8.14 (d, J=8.2 Hz, 1H), 8.33 (d, J=7.5 Hz, 1H), 9.15 (d,J=8.2 Hz, 1H); ¹³C NMR (75.5 MHz, CDCl₃) δ 14.0, 22.9, 32.8, 33.4, 123.9, 124.3, 124.9, 126.0, 126.6, 127.5, 131.6, 132.1, 132.3, 146.3, 172.9; MS (EI) m/z 228 (53), 185 (100), 157 (36).

1-Propyl-3-(4-butyl-1-naphthoyl)indole, JWH-239. JWH-239 was prepared from 4-butyl-1-naphthoic acid and 1-propylindole by method C. From 0.18 g (0.79 mmol) of 4-butyl-1 -naphthoic acid and 0.15 g (0.95 mmol) of 1-propylindole there was obtained after chromatography (petroleum ether:ether, 9:1) 0.19 g (62%) of JWH-239 as a gum: ¹H NMR (300 MHz, CDCl₃) δ 0.90 (t, J=7.4 Hz, 3H), 1.02 (t, J=7.3 Hz, 3H), 1.46-1.58 (m, 2H), 1.71-1.88 (m, 4H), 3.15 (t, J=7.8 Hz, 2H), 4.04 (t, J=7.1 Hz, 2H), 7.35-7.43 (m, 5H), 7.46-7.50 (m, 2H), 7.52-7.60 (m, 2H), 8.14 (d, J=8.3 Hz, 1H), 8.27 (d, J=8.2 Hz, 1H); ¹³C NMR (75.5 MHz, CDCl₃) δ 11.3, 14.0, 22.9, 23.1, 32.9, 33.1, 48.7, 109.9, 117.6, 122.7, 122.9, 123.5, 124.0, 124.5, 125.7, 126.0, 126.2, 126.7, 127.0, 131.2, 132.1, 137.0, 137.4, 137.9, 141.3, 192.3; MS (EI) m/z 369 (100), 352 (70), 340 (53); HRMS Calcd for C₂₆H₂₇NO: 369.2093, Found 369.2094.

1-Pentyl-3-(4-butyl-1-naphthoyl)indole, JWH-240. JWH-240 was prepared from 4-butyl-1-naphthoic acid and 1-pentylindole by method C. From 0.17 g (0.75 mmol) of 4-butyl-1-naphthoic acid and 0.17 g (0.89 mmol) of 1-pentylindole there was obtained after chromatography (petroleum ether:ether, 9:1) 0.17 g (58%) of JWH-240 as a gum: ¹H NMR (300 MHz, CDCl₃) δ 0.87 (t, J=6.8 Hz, 3H), 1.03 (t, J=7.3 Hz, 3H), 1.24-1.35 (m, 4H), 1.47-1.59 (m, 2H), 1.76-1.86 (m, 4H), 3.16 (t, J=7.8 Hz, 2H), 4.06 (t, J=7.2 Hz, 2H), 7.34-7.42 (m, 5H), 7.45-7.53 (m, 2H), 7.56-7.60 (m, 2H), 8.15 (d, J=8.4 Hz, 1H), 8.28 (d, J=8.3 Hz, 1H); ¹³C NMR (75.5 MHz, CDCl₃) δ 13.8, 14.0, 22.1, 22.9, 28.8, 29.4, 32.9, 33.1, 47.0, 109.9, 117.6, 122.7, 122.9, 123.4, 124.0, 124.5, 125.7, 126.0, 126.1, 126.7, 127.0, 131.2, 132.1, 137.0, 137.4, 137.8, 141.3, 192.3; MS (EI) m/z 397 (100), 380 (59), 340 (82); HRMS Calcd for C₂₉H₃₁NO: 397.2406, Found 397.2400.

2-Methyl-1-propyl-3-(4-butyl-1-naphthoyl)indole, JWH-241. JWH-241 was prepared from 4-butyl-1-naphthoic acid and 2-methyl-1-propylindole by method C. From 0.18 g (0.79 mmol) of 4-butyl-1-naphthoic acid and 0.16 g (0.95 mmol) of 2-methyl-1-propylindole there was obtained after chromatography (petroleum ether:ether, 9:1) 0.18 g (62%) of JWH-241 as a gum: ¹H NMR (300 MHz, CDCl₃) δ 0.97-1.03 (m, 6H), 1.44-1.56 (m, 2H), 1.77-1.90 (m, 4H), 2.49 (s, 3H), 3.16 (t,J=7.7 Hz, 2H), 4.13 (t,J=7.9 Hz, 2H), 7.02 (d,J=7.4 Hz, 1H), 7.16-7.25 (m, 2H), 7.30-7.36 (m, 2H), 7.42-7.56 (m, 3H), 8.13 (d, J=8.5 Hz, 1H), 8.25 (d, J=8.3 Hz, 1H); ¹³C NMR (75.5 MHz, CDCl₃) δ 11.4, 12.5, 14.0, 22.8, 22.9, 32.9, 33.1, 44.8, 109.4, 115.0, 121.3, 121.7, 122.1, 124.0, 125.0, 125.7, 125.9, 126.2, 126.4, 127.1, 130.8, 132.2, 136.1, 138.8, 141.3, 145.4, 193.7; MS (EI) m/z 383 (100), 366 (34), 326 (95); Calcd for C₂₇H₂₉NO: 383.2249, Found 383.2246.

2-Methyl-1-pentyl-3-(4-butyl-1-naphthoyl)indole, JWH-242. JWH-242 was prepared from 4-butyl-1-naphthoic acid and 2-methyl-1-pentylindole by method C. From 0.17 g (0.75 mmol) of 4-butyl-1-naphthoic acid and 0.18 g (0.89 mmol) of 2-methyl-1-pentylindole there was obtained after chromatography (petroleum ether:ether, 9:1) 0.17 g (56%) of JWH-242 as a gum: ¹H NMR (300 MHz, CDCl₃) δ 0.92 (t, J=6.9 Hz, 3H), 1.00 (t, J=7.3 Hz, 3H), 1.39-1.45 (m, 4H), 1.47-1.55 (m, 2H), 1.74-1.84 (m, 4H), 2.48 (s, 3H), 3.16 (t, J=7.7 Hz, 2H), 4.12 (t, J=7.6 Hz, 2H), 7.02 (t, J=7.4 Hz, 1H), 7.16-7.26 (m, 2H), 7.32-7.36 (m, 2H), 7.40-7.56 (m, 3H), 8.14 (d, J=8.5 Hz, 1H), 8.18 (d, J=8.5 Hz, 1H); ¹³C NMR (75.5 MHz, CDCl₃) δ 12.5, 13.9, 14.0, 22.4, 22.8, 29.1, 29.3, 32.9, 33.1, 43.3, 109.3, 121.3, 121.8, 122.1, 124.0, 125.1, 125.7, 125.9, 126.2, 126.4; MS (EI) m/z 411 (89), 396 (60), 354 (100); Calcd for C₂₉H₃₃NO: 411.2562, Found 411.2563.

1-Propyl-3-(7-methyl-1-naphthoyl)indole, JWH-076 (Method A). JWH-076 was prepared from 1-propylindole and 7-methyl-1-naphthoyl chloride by method B. From 0.186 g (0.65 mmol) of 3-(7-methyl-1-naphthoyl)indole and 0.5 mL (5.5 mmol) of 1-bromopropane there was obtained after chromatography (petroleum ether:ethyl acetate, 1:3) 0.207 g (97%) of JWH-076 as a tan gum: ¹H NMR (300 MHz, CDCl₃) δ 0.90 (t, J=7.3 Hz, 3H), 1.85 (q, J=7.3 Hz, 2H), 2.44 (s, 3H), 4.05 (t, J=7.2 Hz, 2H), 7.35-7.47 (m, 6H), 7.62 (d, J=6.8 Hz, 1H), 7.81 (d, J=8.3 Hz, 1H), 7.92 (d, J=8.1 Hz, 1H), 7.98 (s, 1H), 8.52 (t, J=5.4 Hz, 1H); ¹³C NMR (75.5 MHz, CDCl₃) δ 11.3, 21.9, 23.1, 48.7, 110.0, 117.6, 122.8, 123.0, 123.5, 124.8, 125.9, 127.0, 127.9, 128.6, 129.7, 130.9, 132.0, 136.7, 137.0, 138.0, 138.4, 192.3; MS (EI) m/z 327 (82), 310 (43), 298 (58), 186 (100).

7-Ethyl-1-naphthoic Acid (24). To a mixture of 9.56 g of AlCl₃(64 mmol) and 4.05 mL of acetyl chloride (57 mmol) was added, with stirring, 21.3 mL of 1,2-dichloroethane. To this light brown solution was added dropwise a solution of 10.0 g (64 mmol) of 2-ethylnaphthalene in 7.1 mL of 1,2-dichloroethane. The reaction mixture was allowed to stand at ambient temperature for 18 h, diluted with dichloromethane, and shaken with dilute aqueous HCl. After washing with saturated aqueous NaHCO₃ and water, the organic phase was dried (MgSO₄) and the solvents were removed in vacuo. Chromatography (petroleum ether:ethyl acetate, 95:5) gave 9.88 g (86%) of 1-acetyl-7-ethylnaphthalene (26) as a colorless oil: ¹H NMR (300 MHz, CDCl₃) δ 1.33 (t, J=7.6 Hz, 3H), 2.72 (s, 3H), 2.83 (q, J=7.5 Hz, 2H), 7.39-7.50 (m, 2H), 7.79 (d, J=8.4 Hz, 1H), 8.56 (s, 1H); ¹³C NMR (75.5 MHz, CDCl₃) δ 15.5, 29.5, 30.0, 123.4, 123.8, 127.5, 128.3, 128.7, 130.3, 132.4, 132.7, 134.6, 144.3, 202.1.

A solution of 8.7 g (44 mmol) of iodine in 15 mL of pyridine was added to 8.70 g (44 mmol) of 1-acetyl-7-ethylnaphthalene, and the solution was stirred at 100° C. for 30 min. After cooling to ambient temperature the precipitated pyridinium salt was suspended in methanol and collected by filtration. After drying in vacuo at 45° C. there was obtained 23.5 g of salt 27 as a brown solid, which was added to 250 mL of water without further purification and 20 g of solid NaOH were added. The reaction mixture was heated at reflux for 2 h, cooled to ambient temperature and acidified with 10% aqueous HCl to give 7.56 g of acid 24 as dark purple crystals. Recrystallization from aqueous methanol gave 5.74 g (65%) of product as a brown solid. For further purification, to a solution of 1.0 g of acid in 15 mL of methanol was added 3 mL of H₂SO₄ and the reaction mixture was heated at reflux for 2 h. The reaction mixture was concentrated in vacuo, diluted with ether, washed with water and dried (MgSO₄). The solvent was removed in vacuo to give a brown oil, which was chromatographed (petroleum ether:ethyl acetate, 95:5) to give 0.42 g (42%) of ester as a colorless oil: ¹H NMR (300 MHz, CDCl₃) δ 1.29 (t, J=7.6 Hz, 3H), 2.84 (q, J=7.3 Hz, 2H), 3.98 (s, 3H), 7.39 (t, J=7.6 Hz, 1H), 7.38 (d, J=8.0 Hz, 1H), 7.77 (d, J=8.4 Hz, 1H), 7.94 (d, J=8.1 Hz, 1H), 8.07 (d, J=7.7 Hz, 1H), 8.74 (s, 1H); ¹³C NMR (75.5 MHz, CDCl₃) δ 15.5, 29.5, 51.9, 123.5, 126.3, 127.2, 128.4, 130.2, 131.6, 132.3, 133.0, 143.9, 168.0.

To a suspension of 1.66 g (8.4 mmol) of ester in 50 mL of water was added 10 g of KOH and the mixture was heated at reflux for 18 h. After acidification with conc. HCl to pH 5 the precipitated solid was filtered out to give, after drying in vacuo, 1.32 g (80%) of 7-ethyl-1-naphthoic acid (24) as an off white solid, which was used in subsequent steps without further purification. Recrystallizatiion from petroleum ether gave fine white needles: mp 117-120° C. (lit. mp 126° C., ref. 49): ¹H NMR (300 MHz, CDCl₃) δ 1.37 (t, J=7.5 Hz, 3H), 2.90 (q, J=7.5 Hz, 2H), 7.44-7.51 (m, 2H), 7.85 (d, J=8.4 Hz, 1H), 8.06 (d, J=8.2 Hz, 1H), 8.38 (dd, J=1.0, 7.2 Hz, 1H), 8.88 (s, 1H).

1-Propyl-3-(7-ethyl-1-naphthoyl)indole, JWH-235. JWH-235 was prepared from 7-ethyl-1-naphthoic acid by method B. From 0.150 g (0.75 mmol) of 7-ethyl-1-naphthoic acid and 0.08 g (0.50 mmol) of 1-propylindole there was obtained 0.150 g (88%) of JWH-235 as a pale brown oil after chromatography (petroleum ether:ethyl acetate, 95:5): ¹H NMR (300 MHz, CDCl₃) δ 0.83 (t, J=7.3 Hz, 3H), 1.21 (t, J=7.5 Hz, 3H), 1.70-1.83 (m, 2H), 2.72 (q, J=7.5 Hz, 2H), 3.95 (t, J=7.1 Hz, 2H), 7.20-7.38 (m, 5H), 7.42 (d, J=7.2 Hz, 1H), 7.58 (dd, J=7.2, 0.8 Hz, 1H), 7.80 (d, J=8.3 Hz, 1H), 7.88 (d, J=8.3 Hz, 1H), 8.01 (s, 1H), 8.53-8.56 (m 1); ¹³C NMR (75.5 MHz, CDCl₃) δ 11.2, 15.6, 22.9, 29.2, 48.6, 110.0, 117.4, 122.7, 122.8, 123.4, 123.5, 123.6, 125.9, 126.9, 127.3, 128.1, 129.6, 130.9, 132.2, 137.0, 138.0, 138.4, 142.8, 192.1; MS (EI) m/z 341 (100), 312 (80); HRMS Calcd for C₂₄H₂₃NO: 341.1760, Found 341.1779.

1-Pentyl-3-(7-ethyl-1-naphthoyl)indole, JWH-234. JWH-234 was prepared from 7-ethyl-1-naphthoic acid and 1-pentylindole by method B. From 0.150 g (0.75 mmol) of 7-ethyl-1-naphthoic acid and 0.094 g (0.5 mmol) of 1-pentylindole there was obtained after chromatography (petroleum ether:ethyl acetate, 95:5) 0.100 g (54%) of JWH-234 as a pale brown oil: ¹H NMR (300 MHz, CDCl₃) δ 0.85 (t, J=7.0 Hz, 3H), 1.21-1.46 (m, 7H), 1.75-1.84 (m, 2H), 2.73 (q, J=7.6 Hz, 2H), 4.05 (t, J=7.2 Hz, 2H), 7.34-7.41 (m, 5H), 7.45 (d, J=7.2 Hz, 1H), 7.61 (dd, J=7.2, 1.0 Hz, 1H), 7.83 (d, J=8.3 Hz, 1H), 7.92 (d, J=8.3 Hz, 1H), 8.00 (s, 1H), 8.51-8.53 (m,1H); ¹³C NMR (75.5 MHz, CDCl₃) δ 13.8, 15.7, 22.1, 28.7, 29.3, 29.5, 47.1, 109.9, 117.6, 122.8, 122.9, 123.5, 123.6, 123.7, 126.0, 127.0, 127.4, 128.1, 129.7, 131.0, 132.3, 137.0, 137.9, 138.5, 143.0, 192.2; MS (EI) m/z 369 (75), 352 (40), 312 (50), 207 (100); HRMS Calcd for C₂₆H₂₇NO: 369.2091, Found 369.2093.

2-Methyl-1-propyl-3-(7-ethyl-1-naphthoyl) indole, JWH-236. JWH-236 was prepared from 7-ethyl-1-naphthoic acid and 2-methyl-1-propylindole by method B. From 0.150 g (0.75 mmol) of 7-ethyl-1-naphthoic acid and 0.087 g (0.5 mmol) of 2-methyl-1-propylindole there was obtained after chromatography (petroleum ether:ethyl acetate, 95:5) 0.163 g (92%) of JWH-236 as a pale brown oil: ¹H NMR (300 MHz, CDCl₃) δ 0.98 (t, J=7.3 Hz, 3H), 1.21 (t, J=7.6 Hz, 3H), 1.76-1.88 (m, 2H), 2.47 (s, 3H), 2.72 (q, J=7.6 Hz, 2H), 4.08 (t, J=7.4 Hz, 2H), 7.00 (t, J=7.3 Hz, 1H), 7.17 (t, J=7.3 Hz, 1H), 7.24 (d, J=7.6 Hz, 1H), 7.31 (d, J=8.2 Hz, 1H), 7.36-7.51 (m, 2H), 7.52 (d, J=6.0 Hz, 1H), 7.83 (d, J=8.2 Hz, 1H), 7.92 (d, J=8.2 Hz, 1H), 7.96 (s, 1H); ³C NMR (75.5 MHz, CDCl₃) 811.4, 12.5, 15.5, 22.9, 29.2, 44.8, 109.4, 114.9, 121.3, 121.8, 122.1, 123.4, 124.0, 126.0, 127.1, 127.3, 128.1, 129.8, 130.6, 132.3, 136.1, 139.7, 142.9, 145.5, 193.6; MS (EI) m/z 355 (100), 338 (40); HRMS Calcd for C₂₅H₂₅NO: 355.1936, Found 355.1936.

2-Methyl-1-pentyl-3-(7-ethyl-1-naphthoyl)indole, JWH-262. JWH-262 was prepared from 7-ethyl-1-naphthoic acid and 2-methyl-1-pentylindole by method B. From 0.150 g (0.75 mmol) of 7-ethyl-1-naphthoic acid and 0.100 g (0.5 mmol) of 1-pentylindole there was obtained after chromatography (petroleum ether:ethyl acetate, 95:5) 0.167 g (87%) of JWH-262 as a pale brown oil: ¹H NMR (300 MHz, CDCl₃) δ 0.89 (t, J=6.9 Hz, 3H), 1.21 (t, J=7.6 Hz, 3H),1.26-1.43 (m, 4H), 1.74-1.79 (m, 2H), 2.47 (s, 3H), 2.71 (q, J=7.6 Hz, 2H), 4.09 (t, J=7.5 Hz, 2H), 6.99 (t, J=7.3 Hz, 1H), 7.16 (t, J=7.3 Hz, 1H), 7.24 (d, J=8.0 Hz, 1H), 7.30 (d, J=8.0 Hz, 1H), 7.36-7.48 (m, 2H), 7.52 (dd, J=7.1, 1.2 Hz, 1H), 7.83 (d, J=8.4 Hz, 1H), 7.91 (d, J=8.4 Hz, 1H), 7.97 (s, 1H); ¹³C NMR (75.5 MHz, CDCl₃) δ 12.5, 13.9, 15.5, 22.3, 29.0, 29.3, 43.3, 53.3, 109.3, 114.9, 121.1, 121.7, 122.1, 123.4, 124.0, 126.0, 127.1, 127.2, 128.1, 129.7, 130.5, 132.5, 135.9, 139.6, 142.9, 145.4, 193.6; MS (EI) m/z 383 (100), 368 (50), 297 (50); HRMS Calcd for C₂₇H₂₉NO: 382.2175, Found 382.2175.

1-Propyl-3-(4-ethoxy-1-naphthoyl)indole, JWH-259. JWH-259 was prepared from 4-ethoxy-1-naphthoic acid and 1-propylindole by method B. From 0.13 g (0.6 mmol) of 4-ethoxy-1-naphthoic acid and 0.080 g (0.5 mmol) of 1-propylindole there was obtained after chromatography (petroleum ether:ethyl acetate, 9:1) 0.15 g (86%) of JWH-259 as a gum: ¹H NMR (300 MHz, CDCl₃) δ 0.85 (t, J=7.3 Hz, 3H), 1.55 (t, J=6.9 Hz, 3H), 1.75-1.82 (m, 2H), 3.97 (t, J=7.1 Hz, 2H), 4.20 (q, J=6.9 Hz, 2H), 6.74 (d, J=7.9 Hz, 1H), 7.29-7.37 (m, 4H), 7.45-7.47 (m, 2H), 7.60 (d, J=7.9 Hz, 1H), 8.29-8.32 (m, 1H), 8.34-8.38 (m, 1H), 8.46-8.48 (m, 1H); ¹³C NMR (75.5 MHz, CDCl₃) δ 11.2, 14.7, 23.0, 48.5, 63.8, 102.7, 109.9, 117.6, 122.1, 122.5, 122.7, 123.3, 125.4, 125.5, 125.6, 125.7, 127.2, 127.9, 131.1, 132.1, 136.9, 137.4, 156.3, 191.7; HRMS Calcd for C₂₄H₂₃NO2: 357.1725, Found 357.1728.

1-Pentyl-3-(4-ethoxy-1-naphthoyl)indole, JWH-258. JWH-258 was prepared from 4-ethoxy-1-naphthoic acid and 1-pentylindole by method B. From 0.13 g (0.6 mmol) of 4-ethoxy-1-naphthoic acid and 0.094 g (0.5 mmol) of 1-pentylindole there was obtained after chromatography (petroleum ether:ethyl acetate, 9:1) 0.17 g (90%) of JWH-258 as a gum: ¹H NMR (300 MHz, CDCl₃) δ 0.83 (t, J=6.6 Hz, 3H), 1.26-1.29 (m, 4H), 1.56 (t, J=6.9 Hz, 3H), 1.75-1.82 (m, 2H), 4.03 (t, J=7.1 Hz, 2H), 4.22 (q, J=6.9 Hz, 2H), 6.77 (d, J=7.9 Hz, 1H), 7.22-7.38 (m, 4H), 7.47-7.50 (m, 2H), 7.62 (d, J=7.9 Hz, 1H), 8.29-8.33 (m, 1H), 8.35-8.39 (m, 1H), 8.46-8.48 (m, 1H); ¹³C NMR (75.5 MHz, CDCl₃) δ 13.8, 14.7, 22.1, 28.8, 29.4, 46.9, 63.9, 102.8, 109.9, 117.6, 122.1, 122.5, 122.8, 123.3, 125.4, 125.5, 125.7, 125.8, 127.2, 127.9, 131.1, 132.1, 136.9, 137.3, 156.3, 191.7; HRMS Calcd for C₂₆H₂₇NO₂: 385.2038, Found 385.2042.

2-Methyl-1-propyl-3-(4-ethoxy-1-naphthoyl)indole, JWH-261. JWH-261 was prepared from 4-ethoxy-1-naphthoic acid and 2-methyl-1-propylindole by method B. From 0.13 g (0.6 mmol) of 4-ethoxy-1-naphthoic acid and 0.087 g (0.5 mmol) of 2-methyl-1-propylindole there was obtained after chromatography (petroleum ether:ethyl acetate, 9:1) 0.17 g (90%) of JWH-261 as a gum: ¹H NMR (300 MHz, CDCl₃) δ1.04 (t, J=7.2 Hz, 3H), 1.64 (t, J=6.8 Hz, 3H), 1.84-1.91 (m, 2H), 2.54 (s, 3H), 4.11 (t,J=7.2 Hz, 2H), 4.29 (q,J=6.8 Hz, 2H), 6.82 (d,J=7.9 Hz, 1H), 7.10 (t, J=7.5 Hz, 1H), 7.24 (t, J=7.2 Hz, 1H), 7.30-7.44 (m, 2H), 7.55-7.58 (m, 2H), 7.65 (d, J=7.9 Hz, 1H), 8.38-8.41 (m, 1H), 8.47-8.50 (m, 1H); ¹³C NMR (75.5 MHz, CDCl₃) 611.3, 12.3, 14.6,22.8,44.6,63.6, 103.2, 109.3, 115.1, 121.1, 121.4, 121.8, 122.1, 125.3, 125.4, 125.7, 127.1, 127.3, 128.4, 131.8, 132.0, 135.9, 144.6, 156.4, 193.0; HRMS Calcd for C₂₅H₂₅NO₂: 368.3411, Found 368.3403.

2-Methyl-1-pentyl-3-(4-ethoxy-1-naphthoyl)indole, JWH-260. JWH-260 was prepared from 4-ethoxy-1-naphthoic acid and 2-methyl-1-pentylindole by method B. From 0.13 g (0.6 mmol) of 4-ethoxy-1-naphthoic acid and 0.10 g (0.5 mmol) of 2-methyl-1-pentylindole there was obtained after chromatography (petroleum ether:ethyl acetate, 9:1) 0.18 g (90%) of JWH-260 as a gum: ¹H NMR (300 MHz, CDCl₃) δ 0.89 (t, J=6.9 Hz, 3H), 1.34-1.36 (m, 4H), 1.56 (t, J=6.9 Hz, 3H), 1.74-1.81 (m, 2H), 2.47 (s, 3H), 4.07 (t, J=7.4 Hz, 2H), 4.22 (q, J=6.9 Hz, 2H), 6.75 (d, J=7.9 Hz, 1H), 6.99 (t, J=7.5 Hz, 1H), 7.15 (t, J=8.1 Hz, 1H), 7.22-7.30 (m, 2H), 7.45-7.50 (m, 2H), 7.55 (d, J=7.9 Hz, 1H), 8.26-8.29 (m, 1H), 8.36-8.39 (m, 1H); ¹³C NMR (75.5 MHz, CDCl₃) 812.4, 13.9, 14.7, 22.3, 29.0, 29.3, 43.2, 63.8, 103.2, 109.3, 112.2, 115.2, 121.4, 121.9, 122.1, 125.3, 125.5, 125.7, 127.2, 127.3, 128.5, 131.8, 132.1, 135.9, 144.6, 156.5, 193.1; HRMS Calcd for C₂₇H₂₉NO₂: 399.2193, Found 399.2198.

1-Propyl-3-(2-methoxy-1-naphthoyl)indole, JWH-265 (30). Indole 30 was prepared from 2-methoxy-1-naphthoic acid and 1-propylindole by method B. From 0.150 g (0.75 mmol) of 2-methoxy-1-naphthoic acid and 0.075 g (0.47 mmol) of 1-propylindole there was obtained after chromatography (petroleum ether:ethyl acetate, 7:3) 0.113 g (70%) of 1-propyl-3-(2-methoxy-1-naphthoyl)indole (30) as white crystals: m.p. 117-119° C.; ¹H NMR (300 MHz, CDCl₃) δ 0.77 (t, J=7.4 Hz, 3H), 1.70 (sept, J=7.2 Hz, 2H), 3.79 (s, 3H), 3.86 (t, J=6.8 Hz, 2H), 7.21-7.31 (m, 7H), 7.68-7.71 (m, 1H), 7.75-7.78 (m, 1H), 7.87 (d, J=9.0 Hz, 1H), 8.44 (s, 1H); ¹³C NMR (75.5 MHz, CDCl₃) δ 11.0, 22.8, 48.4, 56.5, 109.9, 113.4, 117.9, 122.5, 122.6, 123.2, 123.7, 124.5, 125.1, 126.3, 126.8, 127.7, 128.6, 130.2, 131.8, 136.9, 137.9, 153.3, 190.7; MS (EI) m/z 343 (100), 326 (50), 172 (43); HRMS Calcd for C₂₃H₂₁NO₂: 343.1572, Found 343.1577.

1-Pentyl-3-(2-methoxy-1-naphthoyl)indole, JWH-267 (32). Indole 32 was prepared from 2-methoxy-1-naphthoic acid and 1-pentylindole by method B. From 0.150 (0.75 mmol) of 2-methoxy-1-naphthoic acid and 0.090 g (0.48 mmol) of 1-pentylindole there was obtained after chromatography (petroleum ether:ethyl acetate, 7:3) 0.107 g (60%) of 32 as a viscous yellow gum: ¹H NMR (300 MHz, CDCl₃) δ 0.81 (t, J=6.9 Hz, 3H), 1.18-1.26 (m, 4H), 1.75 (quintet, J 2 032 7.1 Hz, 2H), 3.86 (s, 3H), 3.99 (t, J=7.2 Hz, 2H), 7.24-7.37 (m,7H), 7.68-7.72 (m,1H), 7.80-7.83 (m, 1H), 7.92 (d, J=9.1 Hz, 1H), 8.41 (s, 1H); ¹³C NMR (75.5 MHz, CDCl₃) δ 22.0, 28.7, 29.2, 47.0, 56.5, 109.9, 113.4, 118.0, 122.6, 122.7, 123.2, 123.8, 124.6, 125.2, 126.4, 126.8, 127.7, 128.6, 130.2, 131.8, 136.9, 137.9, 153.4, 190.8; MS (EI) m/z 37 (100), 354 (61), 200 (55); HRMS Calcd for C₂₅H₂₅NO₂: 371.1885, Found 371.1876.

2-Methyl-1-propyl-3-(2-methoxy-1-naphthoyl)indole, JWH-266 (31). Indole 31 was prepared from 2-methoxy-1-naphthoic acid and 2-methyl-1-propylindole by method B. From 0.150 g (0.75 mmol) of 2-methoxy-1-naphthoic acid and 0.086 g (0.50 mmol) of 2-methyl-1-propylindole there was obtained after chromatography (petroleum ether:ethyl acetate, 7:3) 0.100 g (58%) of 31 as a yellow solid: m.p. 124-125° C.; ¹H NMR (300 MHz, CDCl₃) δ 0.86 (t, J=7.3 Hz, 3H), 1.57-1.73 (m, 2H), 3.73 (s, 3H), 3.92 (t, J=6.7 Hz, 2H), 7.08 (s, 3H), 7.17-7.30 (m, 7H), 7.58-7.60 (m, 1H), 7.74-7.76 (m, 1H), 7.87 (d, J=9.1 Hz, 1H); ¹H NMR (500 MHz, DMSO-d6, 25° C.) δ 0.88 (br s, 3H), 1.61-1.78 (m, 2H), 3.78 (s, 3H), 4.08-4.23 (m, 2H), 7.13 (br s, 1H), 7.24-7.64 (m, 6H), 7.93-7.98 (m, 1H), 8.10 (d, J=9.1 Hz, 1H); ¹H NMR (500 MHz, DMSO-d6, 125° C.) δ 0.88 (t, 6.8 Hz, 3H), 1.71-1.80 (m, 2H), 2.36 (s, 3H), 3.80 (s, 3H), 4.17 (t, J=7.4 Hz, 2H), 6.95 (t, J=7.8 Hz, 1H); 7.14 (td, J=0.5, 8.2 Hz, 1H), 7.32-7.39 (m, 2H), 7.47-7.58 (m, 4H), 7.91-7.96 (m,1H), 8.06 (d, J=9.2 Hz, 1H); ¹³C NMR (75.5 MHz, CDCl₃) δ 11.0, 22.8, 44.4, 56.4, 109.3, 115.2, 121.4, 121.9, 122.2, 123.7, 123.9, 125.2, 126.3, 126.8, 127.7, 128.7, 130.0, 131.0, 135.9, 145.7, 152.8, 191.4; MS (EI) m/z 357 (44), 326 (100); HRMS Calcd for C₂₄H₂₃NO₂: 357.1729, Found 357.1734.

2-Methyl-1-pentyl-3-(2-methoxy-1-naphthoyl)indole, JWH-267 (33). Indole 33 was prepared from 2-methoxy-1-naphthoic acid and 2-methyl-1-pentylindole by method B. From 0.150 g (0.75 mmol) of 2-methoxy-1-naphthoic acid and 0.094 g (0.47 mmol) of 2-methyl-1-pentylindole there was obtained after chromatography (petroleum ether:ethyl acetate, 7:3) 0.109 g (60%) of 33 as a yellow solid: m.p. 39-40° C.; ¹H NMR (300 MHz, CDCl₃) δ 0.81 (t, J=7.0 Hz, 3H), 1.35-1.36 (m, 4H), 1.59-1.63 (m, 2H), 3.83 (s, 3H), 4.08 (t, J=7.0 Hz, 2H), 7.15 (s, 3H), 7.25-7.38 (m, 7H), 7.64-7.67 (m,1H), 7.80-7.84 (m, 1H), 7.94 (d, J=9.0 Hz, 1H); 2-Methyl-1-pentyl-3-(2-methoxy-1-naphthoyl)indole at 25; ¹H NMR (500 MHz, toluene-d8, 25° C.) 80.72 (t, J=7.4 Hz, 3H), 0.87-096 (m, 2H), 0.96-1.07 (m, 2H), 1.18-1.27 (m, 2H), 2.15-2.74 (br, 3H), 3.32 (s, 3H), 3.41 (t, J=6.85 Hz, 2H), 6.88-7.25 (m,7H), 7.60-7.64 (m, 1H), 7.65 (d, J=9.2 Hz, 1H), 7.97 (d, J=7.8 Hz, 1H); ¹H NMR (500 MHz, toluene-d8, 100° C.) d 0.72 (t, J=7.4 Hz, 3H), 0.97-1.11 (m, 4H), 1.30-1.39 (m, 2H), 2.34 (s, 3H), 3.41 (s, 3H), 3.54 (t, J=8.7 Hz, 2H), 6.89 (br s, 1H), 6.95-7.12 (m, 6H), 7.60 (d, J=7.3 Hz, 1H), 7.64 (d, J=10.2 Hz, 1H), 7.90 (d, J=8.2 Hz, 1H); ¹³C NMR (75.5 MHz, CDCl₃) δ 13.9, 22.3, 29.0, 29.2, 43.2, 56.7, 109.3, 113.6, 116.0, 122.1, 122.2, 122.4, 123.9, 124.2, 125.2, 126.3, 127.0, 127.8, 128.9, 130.2, 131.2, 138.6, 152.9, 191.3; MS (EI) m/z 385 (34), 354 (100), 185 (46); HRMS Calcd for C₂₆H₂₇NO₂: 385.2042, Found 385.2040.

6-Methoxy-1-naphthoic acid (35). To a solution of 2.3 g (13.2 mmol) of 6-methoxy-1-naphthol ^{40,41} in 50 mL of pyridine was added dropwise at 0° C. 2.6 mL (15.5 mmol) of trifluoromethanesulfonic anhydride. The solution was warmed to ambient temperature, stirred for 18 h, quenched with water and extracted with ether. The combined ethereal extracts were washed with 10% aqueous HCl until the aqueous solution was acidic. The solution was dried (MgSO4) and the solvent was removed in vacuo. The residue was chromatographed (petroleum ether:ethyl acetate, 12:1) to give 3.4 g (84%) of triflate as a yellow oil which was used in the subsequent step without further purification: ¹H NMR (300 MHz, CDCl₃) δ 3.92 (s, 3H), 7.16 (d, J=2.4 Hz, 1H), 7.27 (dd, J=8.7, 2.4 Hz, 2H), 7.41 (t, J=8.1 Hz, 1H), 7.95 (d, J=9.0 Hz, 1H); ¹³C NMR (75.5 MHz, CDCl₃) δ 55.4, 106.0 115.3, 116.6, 120.7, 120.9, 121.6, 122.4, 125.8, 127.2, 136.5, 145.8, 158.7; MS (EI) m/z 306 (25), 173 (55), 145 (100).

To a solution of 0.96 g (3.18 mmol) of 6-methoxy-1-naphthyl trifluoromethanesulfonate in 30 mL of DMF was added 0.034 g (0.15 mmol) of Pd(OAc)2, 0.040 g (0.010 mmol) of 1,3-bis(diphenylphosphino)propane and 1.20 g of triethylamine. The mixture was flushed with carbon monoxide for 25 min, 3.1 mL of 96% formic acid was added dropwise and the reaction was stirred at ambient temperature for 6 h under an atmosphere of CO, diluted with water and extracted with ethyl acetate. The organic extracts were washed with five portions of brine, followed by two portions of aqueous NaHCO₃. The bicarbonate extracts were combined, cautiously neutralized with 10% aqueous HCl, and extracted with ether. The ethereal extracts were washed with water, dried (MgSO₄) and the solvent was removed in vacuo to give 0.42 g (65%) of acid 35 as a yellow powder. Recrystallization from ethyl acetate:petroleum ether gave pale yellow needles: m.p. 185-186° (lit mp 180-180.5° C., ref. 36); ¹H NMR (300 MHz, CDCl₃) δ 3.89 (s, 3H), 7.29 (dd, J=6.9, 2.4 Hz, 1H), 7.41 (d, J=2.4 Hz, 1H), 7.53 (t, J=7.8 Hz, 1H), 7.98 (d, J=6.1 Hz, 1H), 8.05 (d, J=8.1 Hz, 1H), 8.79 (d, J=9.1 Hz, 1H); ¹³C NMR (75.5 MHz, CDCl₃) δ 55.2, 106.7, 120.0, 125.4, 126.1, 127.1, 127.5, 127.6, 131.8, 135.2, 157.1, 168.8; MS (EI) m/z 202 (100), 159 (35), 109 (50).

1-Propyl-3-(6-methoxy-1-naphthoyl)indole, JWH-163 (Method D). To a solution of 0.18 g (0.90 mmol) of 6-methoxy-1-naphthoic acid (35) in 10 mL of dichloromethane was added dropwise 0.44 ml (5.0 mmol) of oxalyl chloride. The solution was stirred at room temperature for 1 h, then heated at reflux for 1 h. After cooling, the solvent and residual oxalyl chloride were removed in vacuo. The residue was dissolved in 5 mL of toluene and added to a solution of 0.20 g (1.3 mmol) of 1-propylindole in 4 mL of toluene at 0° C. To this solution was added dropwise 0.82 mL (1.5 mmol) of 1.8 M EtAlCl₂. The mixture was allowed to warm to ambient temperature and stirred for 18 h. After quenching with water, the reaction mixture was extracted with ether. The ethereal extracts were washed with water, dried (MgSO₄) and the solvent was removed in vacuo. The residue was chromatographed (petroleum ether:ethyl acetate, 8:1) to give 0.19 g of JWH-163 as a colorless solid: m.p. 139-140° C.; ¹H NMR (300 MHz, CDCl₃) δ 0.87 (t, J=7.3 Hz, 3H), 1.81 (sextet, J=7.3 Hz, 2H), 3.91 (s, 3H), 4.01 (t, J=7.2 Hz, 2H), 7.12 (dd, J=9.0, 2.5 Hz, 1H), 7.18 (d, J=2.5 Hz, 1H), 7.30-7.39 (m, 4H), 7.44-7.52 (m, 2H), 7.82 (dd, J=7.5, 1.6 Hz, 1H), 8.09 (d, J=9.0 Hz, 1H), 8.47-8.50 (m, 1H); ¹³C NMR (75.5 MHz, CDCl₃) δ 11.3,23.0,48.7,55.2, 106.0, 110.0, 117.4, 119.3, 122.7, 122.8, 123.5, 123.6, 125.1, 126.1, 126.9, 127.5, 128.7, 135.1, 137.0, 138.0, 139.0, 157.7, 192.1; Anal. Calcd for C₂₃H₂₁NO₂: C, 80.44; H, 6.16; N, 4.08; Found: C, 80.30; H, 6.17; N, 3.99.

1-Pentyl-3-(6-methoxy-1-naphthoyl)indole, JWH-166. JWH-166 was prepared from 6-methoxy-1-naphthoic acid and 1-pentylindole by method D. From 0.19 g (0.9 mmol) of acid and 0.24 g (1.3 mmol) of 1-pentylindole there was obtained after chromatography (petroleum ether:ethyl acetate, 8:1) 0.29 g (81%) of JWH-166 as an oil: ¹H NMR (300 MHz,CDCl₃) δ 0.85 (t, J=6.9 Hz, 3H), 1.23-1.33 (m, 4H), 1.75-1.85 (m, 2H), 3.98 (s, 3H), 4.05 (t, J=7.3 Hz, 2H), 7.12 (dd, J=9.1, 2.5 Hz, 1H), 7.18 (d, J=2.5 Hz, 1H), 7.33-7.40 (m, 4H), 7.47-7.52 (m, 2H), 7.83 (dd, J=7.4, 1.9 Hz, 1H), 8.10 (d, J=9.1 Hz, 1H), 8.45-8.48 (m, 1H); ¹³C NMR (75.5 MHz, CDCl₃) δ 13.8, 22.1, 28.9, 29.4, 47.1, 55.3, 106.1, 110.0, 117.5, 119.4, 122.8, 122.9, 123.5, 123.7, 125.1, 126.2, 127.0, 127.6, 128.7, 135.1, 137.0, 137.8, 139.0, 157.7, 192.1; MS (EI) m/z 371 (90), 354 (75), 314 (65), 214 (100); HRMS Calcd for C₂₅H₂₅NO₂: 371.1885, Found 371.1885.

2-Methyl-1-propyl-3-(6-methoxy-1-naphthoyl)indole, JWH-151 (42). Indole 42 was prepared from 6-methoxy-1-naphthoic acid and 2-methyl-1-propylindole by method D. From 0.19 g (0.9 mmol) of acid and 0.22 g (1.3 mmol) of 2-methyl-1-propylindole there was obtained after chromatography (petroleum ether:ethyl acetate, 8:1) 0.19 g (56%) of 42 as an oil: ¹H NMR (300 MHz, CDCl₃) δ 0.99 (t, J=7.5 Hz, 3H), 1.79-1.86 (m, 2H), 2.47 (s, 3H), 3.92 (s, 3H), 4.09 (t, J=7.5 Hz, 2H), 6.99 (t, J=7.3 Hz, 1H), 7.09 (dd, J=9.0, 2.7 Hz, 1H), 7.14-7.20 (m, 3H), 7.30 (d, J=7.8 Hz, 1H), 7.40-7.49 (m 2), 7.85 (d, J=7.5 Hz, 1H), 8.00 (d, J=9.0 Hz, 1H); ¹³C NMR (75.5 MHz, CDCl₃) 611.4, 12.5,22.9,44.8, 55.2, 106.1, 109.4, 114.9, 119.5, 121.2, 121.8, 122.1, 123.4, 125.7, 127.1, 127.2, 128.7, 135.2, 136.1, 140.5, 145.5, 157.7, 193.5; MS (EI) m/z 357 (100), 340 (40), 207 (98); HRMS Calcd for C₂₄H₂₃NO₂: 357.1729, Found 357.1721.

2-Methyl-1-pentyl-3-(6-methoxy-1-naphthoyl)indole, JWH-153. JWH-153 was prepared from 6-methoxy-1-naphthoic acid and 2-methyl-1-pentylindole by method D. From 0.20 g (1.0 mmol) of acid and 0.24 g (1.2 mmol) of 2-methyl-1-pentylindole there was obtained after chromatography (petroleum ether:ethyl acetate, 12:1) 0.23 g (60%) of JWH-153: m.p. 114.5-116° C.; ¹H NMR (300 MHz, CDCl₃) δ 0.90 (t, J=6.9 Hz, 3H), 1.35-1.38 (m, 4H), 1.74-1.79 (m, 2H), 2.46 (s, 3H), 3.92 (s, 3H), 4.10 (t, J=7.6 Hz, 2H), 6.98 (t, J=6.6 Hz, 1H), 7.08 (dd, J=9.3, 2.7 Hz, 1H), 7.13-7.20 (m 3), 7.29 (d, J=8.4 Hz, 1H), 7.39-7.48 (m, 2H), 7.84 (d, J=7.8 Hz, 1H), 8.98 (d, J=9.0 Hz, 1H); ¹³C NMR (75.5 MHz, CDCl₃) δ 12.4, 13.8, 22.3, 29.0, 29.3, 43.3, 55.2, 106.1, 109.4, 114.9, 119.5, 121.2, 121.8, 122.1, 123.4, 125.6, 125.7, 127.1, 127.2, 128.7, 135.1, 136.0, 140.4, 145.4, 157.7, 193.4; Anal. Calcd for C₂₆H₂₇NO₂: C, 81.01; H, 7.06; N, 3.63; Found: C, 80.80; H, 7.18; N, 3.62.

7-Methoxy-1-naphthoic acid (36). The trifluoromethanesulfonate ester of 7-methoxy-1-naphthol was prepared by the procedure employed for the preparation of the triflate of 6-methoxy-1-naphthol. From 2.30 g (15.5 mmol) of 7-methoxy-1-naphthol (40) ^{40,41} there was obtained after chromatography (petroleum ether:ethyl acetate, 12:1) 3.40 g (84%) of triflate as a colorless oil: ¹H NMR (300 MHz, CDCl₃) δ 3.91 (s, 3H), 7.16 (d, J=2.4 Hz, 1H), 7.27 (dd, J=8.7, 2.4 Hz, 2H), 7.41 (t, J=8.1 Hz, 1H), 7.71 (d, J=8.2 Hz, 1H), 7.95 (d, J=9.0 Hz, 1H); ¹³C NMR (75.5 MHz, CDCl₃) 655.4, 106.0, 115.3, 116.6, 120.7, 120.9, 121.6, 122.4, 125.8, 127.2, 136.5, 145.8, 158.7; MS (EI) m/z 306 (25), 173 (55), 145 (100).

Acid 36 was prepared from 7-methoxy-1-naphthyl trifluoromethanesulfonate by the procedure used for the preparation of acid 35. From 0.96 g (3.18 mmol) of triflate there was obtained, after recrystallization from ethyl acetate:petroleum ether, 0.35 g (54%) of acid 36: m.p. 185-186° (lit mp 169-170° C. ref. 37); ¹H NMR (300 MHz, CDCl₃) δ 3.89 (s, 3H), 7.29 (dd, J=6.9, 2.4 Hz, 1H), 7.41 (d, J=2.4 Hz, 1H), 7.53 (t, J=7.8 Hz, 1H), 7.98 (d, J=6.1 Hz, 1H), 8.05 (d, J=8.1 Hz, 1H), 8.79 (d, J=9.1 Hz, 1H); ¹³C NMR (75.5 MHz, CDCl₃) δ 55.2, 106.7, 120.0, 125.4, 126.1, 127.1, 127.5, 127.6, 131.8, 135.2, 157.1, 168.8; MS (EI) m/z 202 (100), 159 (35), 109 (50).

1-Propyl-3-(7-methoxy-1-naphthoyl)indole JWH-165. JWH-165 was prepared from 7-methoxy-1-naphthoic acid and 1-propylindole by method D. From 0.18 g (0.9 mmol) of acid and 0.24 g (1.2 mmol) of 1-propylindole there was obtained after chromatography (petroleum ether:ethyl acetate, 8:1) 0.19 g (64%) of JWH-165: ¹H NMR (300 MHz, CDCl₃) δ 0.90 (t, J=8.1 Hz, 3H), 1.85 (q, J=7.2 Hz, 2H), 3.82 (s, 3H), 4.05 (t, J=7.2 Hz, 2H), 7.17 (dd, J=8.9, 2.4 Hz, 1H), 7.24-7.41 (m, 5H), 7.61-7.65 (m, 2H), 7.78 (d, J=8.9 Hz, 1H), 7.88 (d, J=8.1 Hz, 1H), 8.47-8.50 (m, 1H); ¹³C NMR (75.5 MHz, CDCl₃) δ 11.3, 23.1, 48.7, 55.3, 103.9, 110.0, 117.5, 119.4, 122.1, 122.7, 122.9, 123.5, 127.1, 129.4, 129.6, 130.0, 132.1, 137.0, 137.4, 137.8, 158.4, 192.2; MS (EI) m/z 343 (100), 326 (60), 314 (55), 207 (85); HRMS Calcd for C₂₃H₂₁NO₂: 343.1572, Found 343.1581.

1-Pentyl-3-(7-methoxy-1-naphthoyl)indole, JWH-164. JWH-164 was prepared from 7-methoxy-1-naphthoic acid and 1-pentylindole by method D. From 0.19 g (1.0 mmol) of acid and 0.24 g (1.2 mmol) of 1-pentylindole there was obtained after chromatography (petroleum ether:ethyl acetate, 8:1) 0.21 g (66%) of JWH-164: ¹H NMR (300 MHz, CDCl₃) δ 0.83 (t, J=7.5 Hz, 3H), 1.18-1.32 (m, 4H), 1.77-1.86 (m, 2H), 3.82 (s, 3H), 4.08 (t, J=7.2 Hz, 2H), 7.18 (dd, J=9.0, 2.5 Hz,1H), 7.34-7.42 (m, 5H), 7.61-7.65 (m, 2H), 7.79 (d, J=9.0 Hz, 1H), 7.89 (d, J=8.1 Hz, 1H), 8.46-8.51 (m, 1H); ¹³C NMR (75.5 MHz, CDCl₃) δ 13.8, 22.1, 28.9, 29.5, 47.1, 55.3, 103.9, 110.0, 117.5, 119.4, 122.1, 122.2, 122.7, 122.9, 123.5, 127.1, 129.4, 129.6, 130.0, 132.1, 137.0, 137.4, 137.7, 158.4, 192.2; MS (EI) m/z 371 (100), 314 (55), 214 (45), 207 (65); HRMS Calcd for C₂₅H₂₅NO₂: 371.1885, Found 371.1893.

2-Methyl-1-propyl-3-(7-methoxy-1-naphthoyl)indole, JWH-160. JWH-160 was prepared from 7-methoxy-1-naphthoic acid and 2-methyl-1-propylindole by method D. From 0.18 g (0.9 mmol) of acid and 0.24 g (1.2 mmol) of 2-methyl-1-propylindole there was obtained after chromatography (petroleum ether:ethyl acetate, 8:1) 0.20 g (66%) of JWH-160 as a viscous oil: ¹H NMR (300 MHz, CDCl₃) δ 0.99 (t, J=7.3 Hz, 3H), 1.77-1.90 (m, 2H), 2.50 (s, 3H), 3.79 (s, 3H), 4.10 (t, J=7.5 Hz, 2H), 7.03 (t, J=7.8 Hz, 1H), 7.16-7.24 (m, 3H), 7.32 (t, J=7.1 Hz, 2H), 7.54-7.60 (m, 2H), 7.80 (d, J=8.9 Hz, 1H), 7.90 (d, J=7.8 Hz, 1H); ¹³C NMR (75.5 MHz, CDCl₃) d 11.4, 12.5, 22.9, 44.8,55.2, 103.7, 109.4, 115.3, 119.3, 121.2, 121.7, 122.1, 122.6, 127.2, 127.4, 129.5, 129.7, 130.1, 131.7, 136.1, 138.5, 145.1, 158.5, 193.8; MS (EI) m/z 357 (100), 340 (40), 300 (15); HRMS Calcd for C₂₄H₂₃NO₂: 357.1729, Found 357.1720.

2-Methyl-1-pentyl-3-(7-methoxy-1-naphthoyl)indole, JWH-159. JWH-159 was prepared from 7-methoxy-1-naphthoic acid and 2-methyl-1-pentylindole by method D. From 0.18 g (0.9 mmol) of acid and 0.24 g (1.2 mmol) of 2-methyl-1-pentylindole there was obtained after chromatography (petroleum ether:ethyl acetate, 8:1) 0.19 g (43%) of JWH-159 as a viscous oil: ¹H NMR (300 MHz, CDCl₃) δ 0.91 (6.9, 3H), 1.18-1.26 (m, 4H), 1.80 (t, J=7.3 Hz, 2H), 2.50 (s, 3H), 3.79 (s, 3H), 4.12 (t, J=7.5 Hz, 2H), 7.01 (t, J=7.8 Hz, 1H), 7.15-7.24 (m, 3H), 7.32 (t, J=7.8 Hz, 2H), 7.55-7.59 (m, 2H), 7.80 (d, J=8.9 Hz, 1H), 7.89 (d, J=7.9 Hz, 1H); ¹³C NMR (75.5 MHz, CDCl₃) δ 12.5, 13.9,22.4,29.1,29.4, 43.4,55.3, 103.7, 109.4, 115.0, 119.3, 121.2, 121.7, 122.1, 122.6, 127.2, 127.4, 129.5, 129.7, 130.1, 131.7, 136.0, 138.5, 145.0, 158.5, 193.8; MS (EI) m/z 385 (45), 328 (82), 207 (100); HRMS Calcd for C₂₆H₂₇NO₂: 385.2041, Found 385.2039.

Receptor Binding Assays.

1. CB₁ Assay. [³H] CP-55,940 (KD=690 nM) binding to P2 membranes was conducted as described elsewhere,⁵⁰ except whole brain (rather than cortex only) was used. Displacement curves were generated by incubating drugs with 1 nM of [³H] CP-55,940. The assays were performed in triplicate, and the results represent the combined data from three individual experiments.

2.CB₂ Assay. Human embryonic kidney 293 cells were maintained in Dulbecco's modified Eagle's medium (DMEM) with 10% fetal clone II (HyClone, Logan Utah) and 5% CO₂ at 37° C. in a Form a incubator. Cell lines were created by transfection of CB₂ pcDNA3 into 293 cells by the Lipofectamine reagent (Life Technologies, Gaithersburg, Md.). The human CB₂ cDNA was a gift. Stable transformants were selected in growth medium containing geneticin (1 mg/mL, reagent, Life Technologies, Gaithersburg, Md.). Colonies of about 500 cells were picked (about 2 weeks post transfection) and allowed to expand, then tested for expression of receptor mRNA by northern blot analysis. Cell lines containing moderate to high levels of receptor mRNA were tested for receptor binding properties. Transfected cell lines were maintained in DMEM with 10% fetal clone II plus 0.3-0.5 mg/mL geneticin and 5% CO₂ at 37° C. in a Form a incubator.

The current assay is a modification of Compton et al.⁴² Cells were harvested in phosphate-buffered saline containing 1 mM EDTA and centrifuged at 500 g. The cell pellet was homogenized in 10 mL of solution A (50 mM Tris-HCl, 320 mM sucrose, 2 mM EDTA, 5 mM MgCl₂, pH 7.4). The homogenate was centrifuged at 1,600×g (10 min), the supernatant saved, and the pellet washed three times in solution A with subsequent centrifugation. The combined supernatants were centrifuged at 100,000×g (60 min). The (P2 membrane) pellet was resuspended in 3 mL of buffer B (50 mM Tris-HCl, 1 mM EDTA, 3 mM MgCl₂, pH 7.4) to yield a protein concentration of approximately 1 mg/mL. The tissue preparation was divided into equal aliquots, frozen on dry ice, and stored at −70° C. Binding was initiated by the addition of 40-50 μg membrane protein to silanized tubes containing [³H]CP-55,940 (102.9 Ci/mmol) and a sufficient volume of buffer C (50 mM Tris-HCl, 1 mM EDTA, 3 mM MgCl₂, and 5 mg/mL fatty acid free BSA, pH 7.4) to bring the total volume to 0.5 mL. The addition of 1 μM unlabelled CP-55,940 was used to assess nonspecific binding. Following incubation (30° C. for 1 hour), binding was terminated by the addition of 2 mL of ice cold buffer D (50 mM Tris-HCl, pH 7.4, plus 1 mg/mL BSA) and rapid vacuum filtration through Whatman GF/C filters (pretreated with polyethyleneimine (0.1%) for at least 2 hours). Tubes were rinsed with 2 mL of ice cold buffer D, which was also filtered, and the filters subsequently rinsed twice with 4 mL of ice cold buffer D. Before radioactivity was quantitated by liquid scintillation spectrometry, filters were shaken for 1 hr in 5 mL of scintillation fluid.

CP-55,940 and all cannabinoid analogs were prepared by suspension in assay buffer from a 1 mg/mL ethanolic stock without evaporation of the ethanol (final concentration of no more than 0.4%). When anandamide was used as a displacing ligand, experiments were performed in the presence of phenylmethylsulfonyl fluoride (50 μM). Competition assays were conducted with 1 nM [³H]-55,940 or 1 nM [³H]SR141716A and 6 concentrations (0.1 nM to 10 μM displacing ligands). Displacement IC₅₀ values were originally determined by unweighted least-squares linear regression of log concentration-percent displacement data and then converted to Kᵢ values using the method of Cheng and Prusoff.⁵¹ [³S]GTPγS Binding Assays. Materials. All chemicals were from Sigma (St. Louis, Mo.) except the following: [³⁵S]GTPγS (1250 Ci/mmol) was purchased from New England Nuclear Group (Boston, Mass.), GTPγS from Boehringer Mannheim (New York, N.Y.), and DMEM/F-12 from Fischer Scientific (Pittsburgh, Pa.). Whatman GF/B glass fiber filters were purchased from Fisher Scientific (Pittsburgh, Pa.).

Membrane Preparations. Chinese Hamster Ovary (CHO) cells stably expressing the human _{CB2} receptor (CB₂13 CHO) were cultured in a 50:50 mixture of DMEM and Ham F-12 supplemented with 100 U/ml penicillin, 100 U/ml streptomycin, 0.25 mg/ml G418, and 5% fetal calf serum. Cells were harvested by replacement of the media with cold phosphate-buffered saline containing 0.4% EDTA followed by agitation. Membranes were prepared by homogenization of cells in 50 mM Tris-,HCl, 3 mM MgCl₂, 1 mM EGTA, pH 7.4, centrifugation at 50,000×g for 10 minutes at 4° C., and resuspension in the same buffer at 1.5 mg/ml. Membranes were stored at −80° C. until use.

[³⁵S]GTPγS Binding. Prior to assays, samples were thawed on ice, centrifuged at 50,000×g for 10 minutes at 4° C., and resuspended in Assay Buffer (50 mM Tris-HCl (pH 7.4), 3 mM MgCl₂, 0.2 mM EGTA, and 100 mM NaCl). Reactions containing 10 Bg of membrane protein were incubated for 1.5 hr at 30° C. in Assay Buffer containing 10 BM GDP, 0.1 nM [³⁵S]GTPγS, 0.1% bovine serum albumin, and various concentrations of agonist. Nonspecific binding was determined in the presence of 20 BM unlabeled GTPγS. Reactions were terminated by rapid vacuum filtration through GF/B glass fiber filters, and radioactivity was measured by liquid scintillation spectrophotometry at 95% efficiency for ³⁵S.

Data Analysis. All data are reported as the means ± SEM of at least three experiments, each performed in triplicate. Nonlinear regression analysis was conducted by iterative fitting using JMP (SAS for Macintosh). Nonspecific [³⁵S]GTPγS binding was subtracted from all data. Basal [³⁵S]GTPγS binding is defined as specific [³⁵S]GTPγS binding in the absence of drug. Net-stimulated [³⁵S]GTPγS binding is defined as [³⁵S]GTPγS binding in the presence of drug minus basal. Percent stimulation is expressed as (net stimulated [³⁵S]GTPγS binding/basal)×100%.

Molecular Modeling

1. Conformational Analysis. The structures of the N-propyl-C-2H series analogs: JWH-163 and JWH-180; the N-propyl-C-2 methyl series analogs: JWH-151 and JWH189; the N-pentyl-C-2H series analogs: JWH-166, JWH-182, JWH-267; and, the N-pentyl C-2-methyl series analogs: JWH-153, JWH-181 and JWH-268 were built in the Spartan molecular modeling program (V4.1.1; Wavefunction, Inc. Irvine, Calif.). Each structure was minimized using the AM1, semi-empirical method. For each minimized structure, AM1 conformational searches were then performed for rotation about the carbonyl to indole C-3 and carbonyl to naphthoyl C-1 bonds. Coordinate Drive. In order to describe the energy barrier for rotation about the carbonyl oxygen-carbonyl carbon-naphthalene C-1-naphthalene C-2 torsion angle in JWH-268 (33), an AM1 coordinate drive study was undertaken using Spartan. The torsion angle was driven from its global minimum energy value of 99.47° to −80.49° in 10° increments. The single point energies of the lowest energy and highest energy conformers identified during this drive were calculated at the HF 3-21G* level using Spartan.

2. CB₁ Receptor Docking Studies. Amino Acid Numbering System. In the discussion of receptor residues that follows, the amino acid numbering scheme proposed by Ballesteros and Weinstein52 is used. In this numbering system, the most highly conserved residue in each transmembrane helix (TMH) is assigned a locant of 50. This number is preceded by the TMH number and may be followed in parentheses by the sequence number. All other residues in a TMH are numbered relative to this residue. In this numbering system, for example, the most highly conserved residue in TMH 2 of the CB₁ receptor is D2.50(163). The residue that immediately precedes it is A2.⁴⁹(162).

Ligand/CB₁ R* Complex. Because agonists are thought to have higher affinity for the activated form of GPCRs,53 agonist ligands in the work reported here were docked in a model of the activated state (R*) of CB₁. This R* CB₁ model was created by modification of our rhodopsin-based model of the inactive (R) form of CB₁ and guided by the biophysical literature on the R to R* transition. A complete discussion of the creation of both our CB₁ R and R* models can be found in our published work.^{47,54}

A recent CB₁ mutation study has suggested that residues F3.36, W5.43 and W6.48 are part of the binding site of the aminoalkylindole, WIN55212-2.47 These results are consistent with the earlier work of Shire which showed that the TMH4-E2-TMH5 region of CB₁ contains residues critical for the binding of SR141716A and WIN55,212-2.55 Because the indoles studied here are structurally related to the aminoalkylindoles, we chose to dock the indoles studied here in the same region of CB₁ (i.e., the TMH3-4-5-6 region). Each ligand was docked in the CB₁ TMH 3-4-5-6 region using interactive computer graphics. For all analogs studied, the lowest energy s-trans conformer of each ligand was used for docking studies.⁴⁶ The energy of the CB₁+R* TMH bundle/ligand complex was minimized using the AMBER* united atom force field in Macromodel 6.5 (Schrodinger Inc., Portland, Oreg.). A distance dependent dielectric, 8.0 Å extended non-bonded cutoff (updated every 10 steps), 20.0 Å electrostatic cutoff, and 4.0 Å hydrogen bond cutoff were used. The first stage of the calculation consisted of 2000 steps of Polak-Ribier conjugate gradient (CG) minimization in which a force constant of 225 kJ/mol was used on the helix backbone atoms in order to hold the helix backbones fixed, while permitting the side chains to relax. The second stage of the calculation consisted of 100 steps of CG in which the force constant on the helix backbone atoms was reduced to 50 kJ/mol in order to allow the helix backbones to adjust. Stages one and two were repeated with the number of CG steps in stage two incremented from 100 to 500 steps until a gradient of 0.001 kJ/(mole Å2) was reached. Explicit hydrogens were included on all aromatic amino acid residues in order to better simulate aromatic stacking interactions.⁵⁶

Each resultant receptor/ligand complex was analyzed for the presence of hydrogen bonding and aromatic stacking interactions. Aromatic stacking interactions were identified using Burley and Petsko's criteria. These investigators reported that aromatic-aromatic stacking interactions in proteins operate at distances (d) of 4.5 to 7.0 Å between ring centroids. The angle (a) between normal vectors of interacting aromatic rings typically is between 30° and 90°, producing a “tilted-T” or “edge-to-face” arrangement of interacting rings. Residues and/or ligand regions were designated here as participating in an aromatic stacking interaction if they had centroid to centroid distances between 4.5 Å and 7.0 Å. These interactions were further classified as “tilted-T” arrangements if 30°≦α≦90° and as parallel arrangements for a <30°. In interactions where a=0°, arrangements were also identified as offset or not offset, as Hunter and co-workers have shown that off-set parallel stacks are more energetically favorable.⁵⁷

### REFERENCES AND NOTES FOR EXAMPLE 2

(1) Gaoni, Y.; Mechoulam, R. J. Am. Chem. Soc. 1964, 86, 1648.
(2) Razdan, R. K. Pharmacol. Rev. 1986, 38, 75.
(3) Rapaka, R. S.; Makriyannis, A. Structure-Activity Relationships of the Cannabinoids, NIDA Research Monograph 79; National Institute on Drug Abuse, Rockville, Md., 1987.
(4) Mechoulam, R.; Devane, W. A.; Glaser, R. In Marijuana/Cannabinoids: Neurobiology and Neurophysiology; Murphy, L.; Bartke, A. Ed.; CRC Press, Boca Raton 1992; pp 1-33.
(5) Huffman, J. W.; Lainton, J. A. H. Curr. Med. Chem. 1996, 3, 101.
(6) Seltzman, H. H. Curr. Med. Chem. 1999, 6, 685.
(7) Melvin, L. S.; Johnson, M. R.; Herbert, C. A.; Milne, G. M.; Weissman, A. A. J. Med. Chem., 1984, 27, 67.
(8) Johnson, M. R.; Melvin, L. S. In Cannabinoids as Therapeutic Agents; Mechoulam, R., Ed; CRC Press; Boca Raton, Fla., 1986; pp 121-145.
(9) Matsuda, L. A.; Lolait, S. J.; Brownstein, M. J.; Young, A. C.; Bonner, T. H. Nature, 1990,346,561.

(10) Herkenham, M.; Lynn, A. B.; Little, M. D.; Johnson, M. R.; Melvin, L. S.; De Costa, D. R.; Rice, K. C. Proc. Natl. Acad. Sci. USA, 1990, 87, 1932.
(11) Pertwee, R. G. Curr. Med. Chem. 1999, 6,635.
(12)Munro, S.; Thomas. K. L.; Abu-Shar, M. Nature (London) 1993, 365, 61.
(13) Breivogel, C. S.; Griffin, G.; Di Marzo, V.; Martin, B. R. Mol. Pharmacol., 2001, 155.
(14) Bell, M. R.; D'Ambra, T. E.; Kumar, V.; Eissenstat, M. A.; Herrmann, J. L.; Wetzel, J. R.; Rosi, D.; Philion, R. E.; Daum, S. J.; Hlasta, D. J.; Kullnig, R. K.; Ackerman, J. H.; Haubrich, D. R.; Luttinger, D. A.; Baizman, E. R.; Miller, M. S.; Ward, S. J. J. Med. Chem. 1991, 34, 1099.
(15) D'Ambra, T. E.; Estep, K. G.; Bell, M. R.; Eissenstat, M. A.; Josef, K. A.; Ward, J.; Haycock, D. A.; Baizman, E. R.; Casiano, F. M.; Beglin, N.C.; Chippari, S. M.; Grego, J. D.; Kullnig, R. K.; Daley, G. T. J. Med. Chem. 1992, 35, 124.
(16) Compton, D. R.; Gold, L. H.; Ward, S. J.; Balster, R. L.; Martin, B. R. J. Pharmacol. Exp. Ther. 1992, 263,1118.
(17) Showalter, V. M.; Compton, D. R.; Martin, B. R.; Abood, M. E. J. Pharmacol. Exp. Ther. 1996, 278, 989.
(18) Eissenstat, M. A.; Bell, M. R.; D'Ambra, T. E.; Alexander, E. J.; Daum, S. J.; Ackerman, J. H.; Gruett, M.D.; Kumar, V.; Estep, K. G.; Olefirowicz, E. M.; Wetzel, J. R.; Alexander, M. D.; Weaver, J. D.; Haycock, D. A.; Luttinger, D. A.; Casiano, F. M.; Chippari, S. M.; Kuster, J. E.; Stevenson, J. I.; Ward, S. J. J. Med. Chem. 1995, 3094.
(19) Huffman, J. W.; Dai, D.; Martin, B. R.; Martin, B. R.; Compton, D. R. Bioorg. Med. Chem. Lett. 1994, 4, 563.
(20) Wiley, J. L.; Compton, D. R.; Dai, D.; Lainton, J. A. H.; Phillips, M.; Huffman, J. W.; Martin, B. R. J. Pharmacol. Exp. Ther. 1998, 285, 995.
(21) Aung, M. M.; Griffin, G.; Huffman, J. W.; Wu, M.-J.; Keel, C.; Yang, B.; Showalter, V. M.; Abood, M. E.; Martin, B. R. Drug Alcohol Depend. 2000, 60, 133.
(22) Huffman, J. W. Curr. Med. Chem. 1999, 6, 705.
(23) Huffman, J. W. Curr. Pharm. Des. 2000, 6, 1323.
(24) Huffman, J. W.; Mabon, R.; Wu, M.-J.; Lu, J.; Hart, R.; Hurst, D. P., Reggio, P. H.; Wiley, J. L.; Martin, B. R. Bioorg. Med. Chem. 2003, 11, 539.
(25) Okauchi, T.; Itonaga, M.; Minami, T.; Owa, T.; Kitoh, K.; Yoshino, H. Org. Lett. 2000, 2, 1485.
(26) Sergievskaya, S. T.; Safonova, T. S. J. Gen. Chem. USSR Engl. Transl. 1957, 27, 1715.
(27) Wilshire, J. F. K. Aust. J. Chem. 1967, 20, 575.
(28) Cason, J.; Wordie, J. D. J. Org. Chem. 1950, 15, 617.
(29) The details of the synthesis of acids 13-15 are found in the dissertation of Gulay Zengin, Clemson University, May 2003.
(30) Snatzke, G.; Kunde, K. Chem. Ber. 1973, 106, 1341.
(31) King, L. C. J. Am. Chem. Soc. 1944,66,894.
(32) Ahmed, A.; Bragg, R. A.; Clayden, J.; Lai, L. W.; McCarthy, C.; Pink, J. H.; Westlund, N.; Yasin, S. A. Tetrahedron, 1998, 54, 13277.
(33) details to be published elsewhere.
(34) Dai, D. Ph.D. dissertation, Clemson University, 1993.
   (35) Nelson, J. H. Nuclear Magnetic Resonance Spectroscopy; Prentice Hall: Upper Saddle River, N.J., 2003; pp 254-276.
(36) Price, C. C.; Chapin, E. C.; Goldman, A.; Krebs, E.; Shafer, H. M. J. Am. Chem. Soc. 1941, 63, 1857.
(37) Fieser, L. F.; Holmes, H. L. J. Am. Chem. Soc. 1936, 58, 2319.
(38) Gottlieb, L.; Kellner, D.; Loewenthal, H. J. E. Synth. Commun. 1989, 19, 2987.
(39) Cacchi, S.; Morera, E.; Ortar, G. Tetrahedron Lett. 1985, 26, 1109.
(40) Shand, A. J.; Thomson, R. H. Tetrahedron, 1963, 1919.
(41) The details of the preparation of phenols 36 and 37 will be found in the dissertation of J. Lu, Clemson University, 2000.
(42) Compton, D. R.; Rice, K. C.; De Costa, B. R.; Razdan, R. K.; Melvin, L. S.; Johnson, M. R.; Martin, B. R. J. Pharmacol Exp. Ther. 1993, 265, 218.
(43) Selley, D. E.; Stark, S.; Sim, L. J.; Childers, S. R. Life Sci. 1996, 59, 659.
(44) Huffman, J. W.; Liddle, J.; Yu, S.; Aung, M. M.; Abood, M. E.; Wiley, J. L.; Martin, B. R. Bioorg. Med. Chem. 1999, 7, 2905.
(45) Huffman, J. W.; Bushell, S. M.; Miller, J. R. A.; Wiley, J. L.; Martin, B. R. Bioorg. Med. Chem. 2002,10,4119.
(46) Reggio, P. H.: Basu-Dutt, S.; Barnett-Norris, J.; Castro M. T.; Hurst, D. P. Seltzman, H. H.; Roche M. J.; Gilliam A. F.; Thomas B. F.; Stevenson, L. A,; Pertwee R. G.; Abood M. E. J. Med. Chem. 1998, 41, 5177.
(47) McAllister S. D.; Rizvi G.; Anavi-Goffer S.; Hurst D. P.; Barnett-Norris J.; Lynch D. L.; Reggio P. H.; Abood M. E. J Med Chem 2003, 46, 5139.
(48) (a) Shire D.; Calandra B.; Bouaboula M.; Barth F.; Rinaldi-Carmona M.; Casellas P.; Ferrara P. Life Sci. 1999, 65, 627. (b) Shire D.; Calandra B.; Delpech M.; Dumont X.; Kaghad M.; Le Fur G.; Caput D.; Ferrara P. J. Biol. Chem. 1996, 271, 6941.
(49) Baddar, F. G.; Warren, F. L. J. Chem. Soc. 1939, 244.
(50) Martin, B. R.; Compton, D. R.; Thomas, B. F.; Prescott, W. R., Little; P. J., Razdan, R. K.; Johnson, M. R.; Melvin, L. S.; Mechoulam, R.; Ward, S. J. Pharmacol. Biochem. Behav. 1991,40,471.
(51) Cheng, Y. C.; Prusoff, W. H. Biochem. Pharmacol. 1973, 22, 3099.
(52) Ballesteros, J. A.; Weinstein H. In Methods in Neuroscience; Conn, P.M. Sealfon, S.C. Eds.; Academic Press: New York, 1995: Vol. 25, pp 366-428.
(53) Leff, P. Trends Pharmacol. Sci. 1995, 16, 89.
(54) (a) Barnett-Norris, J.; Hurst, D. P.; Lynch, D. L.; Guarnieri, F.; Makriyannis, A.; Reggio P. H. J. Med. Chem. 2002, 45,:3649. (b) Hurst, D. P.; Lynch, D. L.; Barnett-Norris, J.; Hyatt, S. M.; Seltzman, H. H.; Zhong, M.; Song, Z. H.; Nie, J.; Lewis, D.; Reggio, P. H. (2002) Mol. Pharmacol. 2002, 62, 1274.
(55) Shire, D.; Calandra, B.; Bouaboula, M.; Barth, F.; Rinaldi-Carmona, M.; Casellas, P.; Ferrara, P. (1999) Life Sci. 1999, 65, 627.
(56 Burley, S. K.; Petsko, G. A. (1985) Science 1985, 229, 23.
(57) Hunter, C. A.; Singh, J.; Thornton, J. M. (1991) J. Mol. Biol. 1991, 218, 837.

While the invention has been described in terms of its preferred embodiments, those skilled in the art will recognize that the invention can be practiced with modification within the spirit and scope of the appended claims. Accordingly, the present invention should not be limited to the embodiments as described above, but should further include all modifications and equivalents thereof within the spirit and scope of the description provided herein.

1. A compound of general formula 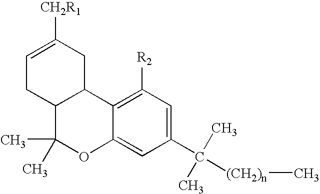
wherein
R₁ and R₂ are H, OH or OCH₃;
n ranges from 0 to 4; and
R₁ and R₂ are not both H.

2. The compound of claim 1 wherein a steric representation of said general formula is 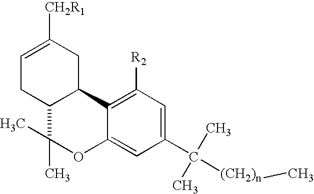
and
R₁ is H or OH;
R₂ is H or OCH₃;
n ranges from 0 to 4; and
R₁ and R₂ are not both H.

3. The compound of claim 1 wherein R₁ is H and R₂ is OCH₃.

4. The compound of claim 1 wherein R₁ is OH and R₂ is H.

5. The compound of claim 1 wherein R₁ is OH and R₂ is OCH₃.

6. A method for selectively binding to CB₂ receptors and not to CB₁ receptors, comprising the step of
exposing said CB₂ receptors and said CB₁ receptors to a compound of general formula 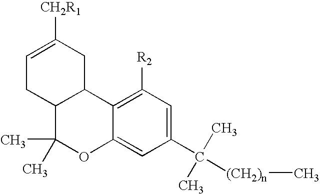
wherein
R₁ and R₂ are H, OH, or OCH₃;
n ranges from 0 to 4; and
R₁ and R₂ are not both H.

7. The method of claim 6 wherein a steric representation of said general formula is 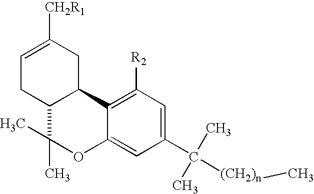
and
R₁ is H or OH;
R₂ is H or OCH₃;
n ranges from 0 to 4; and
R₁ and R₂ are not both H.

8. The method of claim 6 wherein R₁ is H and R₂ is OCH₃.

9. The method of claim 6 wherein R₁ is OH and R₂ is H.

10. The method of claim 6 wherein R₁ is OH and R₂ is OCH₃.

11. The method of claim 6 wherein said compound binds to said CB₂ receptors in an amount sufficient to treat cancer.

12. The method of claim 11, wherein said cancer is a glioma tumor.

13. The method of claim 6 wherein said compound binds to said CB₂ receptors in an amount sufficient to treat pain.

14. The method of claim 13, wherein said pain is inflammatory pain.

15. A method for killing tumor cells, comprising the step of
exposing said tumor cells to a compound of general formula 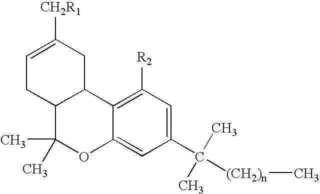
wherein
R₁ and R₂ are H, OH, or OCH₃;
n ranges from 0 to 4; and
R₁ and R₂ are not both H.

16. The method of claim 15, wherein a steric representation of said general formula is 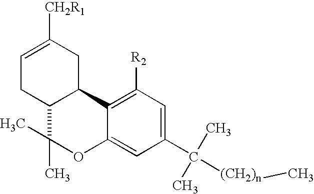
and
R₁ is H or OH;
R₂ is H or OCH₃;
n ranges from 0 to 4; and
R₁ and R₂ are not both H.

17. The method of claim 15 wherein R₁ is H and R₂ is OCH₃.

18. The method of claim 15 wherein R₁ is OH and R₂ is H.

19. The method of claim 15 wherein R₁ is OH and R₂ is OCH₃.

20. The method of claim 15, wherein said tumor cells are glioma tumor cells.

21. A compound of general formula 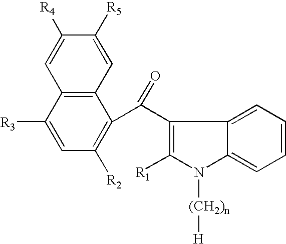
wherein
R₁, R₂, R₃, R₄ and R₅ are H, CH₃, or OCH₃; and
n ranges from 1 to 5.

22. The compound of claim 21, wherein
R₁ is H or CH₃;
R₂ is H or OCH₃;
R₃ is H, CH₃, or OCH₃;
R4 is H or OCH₃; and
R₅ is H or CH₃.

23. The compound of claim 21 wherein: n is 3; R₁, R₃, R₄ and R₅ are H; and R₂ is OCH₃.

24. The compound of claim 21 wherein: n is 3; R₁ is CH₃; R₂ is OCH₃; and R₃, R₄ and R₅ are H.

25. The compound of claim 21 wherein: n is 5; R₁, R₃, R₄ and R₅ are H; and R₂ is OCH₃.

26. The compound of claim 21 wherein: n is 5; R₁ is CH₃, R₂ is OCH₃ and R₃, R₄ and R₅ are H.

27. The compound of claim 21 wherein: n is 3 or 5; R₁, R₂, R₃ and R₅ are H; and R₄ is OCH₃.

28. The compound of claim 21 wherein: n is 3 or 5; R₁ is CH₃; R₂, R₃ and R₅ are H; and R₄ is OCH₃.

29. The compound of claim 21 wherein: n is 3; R₁, R₂, R₄ and R₅ are H; and R₃ is CH₃.

30. The compound of claim 21 wherein: n is 3 or 5; R₁ is CH₃; R₂, R₃ and R₄ are H; and R₅ is CH₃.

31. A method for selectively binding to CB₂ receptors and not to CB₁ receptors, comprising the step of
exposing said CB₂ receptors and said CB₁ receptors to a compound of general formula 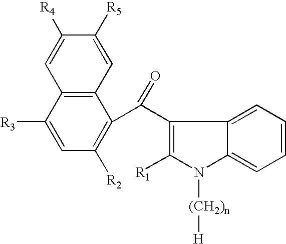
wherein
R₁, R₂, R₃, R₄ and R₅ are H, CH₃, or OCH₃; and
n ranges from 1 to 5.

32. The method of claim 31, wherein
R₁ is H or CH₃;
R₂ is H or OCH₃;
R₃ is H, CH₃, or OCH₃;
R4 is H or OCH₃; and
R₅ is H or CH₃.

33. The method of claim 31 wherein: n is 3; R₁, R₃,R₄ and R₅ are H; and R₂ is OCH₃.

34. The method of claim 31 wherein: n is 3; R₁ is CH₃; R₂ is OCH₃; and R₃, R₄ and R₅ are H.

35. The method of claim 31 wherein: n is 5; R₁, R₃, R₄ and R₅ are H; and R₂ is OCH₃.

36. The method of claim 31 wherein: n is 5; R₁ is CH₃, R₂ is OCH₃ and R₃, R₄ and R₅ are H.

37. The compound of claim 31 wherein: n is 3 or 5; R₁, R₂, R₃ and R₅ are H; and R₄ is OCH₃.

38. The compound of claim 31 wherein: n is 3 or 5; R₁ is CH₃; R₂, R₃ and R₅ are H; and R₄ is OCH₃.

39. The compound of claim 31 wherein: n is 3; R₁, R₂, R₄ and R₅ are H; and R₃ is CH₃.

40. The compound of claim 31 wherein: n is 3 or 5; R₁ is CH₃; R₂, R₃ and R₄ are H; and R₅ is CH₃.

41. The method of claim 31 wherein said compound binds to said CB₂ receptors in an amount sufficient to treat cancer.

42. The method of claim 41, wherein said cancer is a glioma tumor.

43. The method of claim 31 wherein said compound binds to said CB₂ receptors in an amount sufficient to treat pain.

44. The method of claim 43, wherein said pain is inflammatory pain.

45. A method for killing tumor cells, comprising the step of
exposing said tumor cells to a compound of general formula 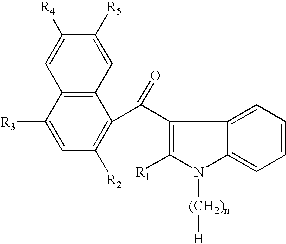
wherein
R₁, R₂, R₃, R₄ and R₅ are H, CH₃, or OCH₃; and
n ranges from 1 to 5.

46. The method of claim 45, wherein
R₁ is H or CH₃;
R₂ is H or OCH₃;
R₃ is H, CH₃, or OCH₃;
R4 is H or OCH₃; and
R₅ is H or CH₃.

47. The method of claim 45 wherein: n is 3; R₁, R₃,R₄ and R₅ are H; and R₂ is OCH₃.

48. The method of claim 45 wherein: n is 3; R₁ is CH₃; R₂ is OCH₃; and R₃, R₄ and R₅ are H.

49. The method of claim 45 wherein: n is 5; R₁, R₃, R₄ and R₅ are H; and R₂ is OCH₃.

50. The method of claim 45 wherein: n is 5; R₁ is CH₃, R₂ is OCH₃ and R₃, R₄ and R₅ are H.

51. The method of claim 45 wherein: n is 3 or 5; R₁, R₂, R₃ and R₅ are H; and R₄ is OCH₃.

52. The method of claim 45 wherein: n is 3 or 5; R₁ is CH₃; R₂, R₃ and R₅ are H; and R₄ is OCH₃.

53. The compound of claim 45 wherein: n is 3; R₁, R₂, R₄ and R₅ are H; and R₃ is CH₃.

54. The compound of claim 45 wherein: n is 3 or 5; R₁ is CH₃; R₂, R₃ and R₄ are H; and R₅ is CH₃.

55. The method of claim 45, wherein said tumor cells are glioma tumor cells.
